(19) United States
(12) Patent Application Publication
(10) Pub. No.: US 2022/0220487 A1
(43) Pub. Date: Jul. 14, 2022

(54) COMBINED EXPRESSION OF TREHALOSE PRODUCING AND TREHALOSE DEGRADING ENZYMES

(71) Applicant: Lallemand Hungary Liquidity Management LLC, Budapest (HU)

(72) Inventors: Trisha Barrett, Bradford, VT (US); Ryan Skinner, Bethel, VT (US); Aaron Argyros, Lebanon, NH (US)

(21) Appl. No.: 17/292,679

(22) PCT Filed: Nov. 13, 2019
(86) PCT No.: PCT/IB2019/059751

§ 371 Date: May 10, 2021

Related U.S. Application Data
(60) Provisional application No. 62/760,649, filed on Nov. 13, 2018.

(51) Int. Cl. C12N 15/52; C12P 19/12; C12N 9/24

(52) U.S. Cl. CPC C12N 15/52; C12N 9/2405; C12P 19/12

(57) ABSTRACT

The present disclosure concerns a recombinant yeast host cell having a first genetic modification for expressing an heterologous trehalase, and a second genetic modification for increasing trehalose production. The present disclosure also concerns a process using the recombinant yeast host cell for making a fermented product, such as ethanol.

### CROSS-REFERENCE TO RELATED APPLICATIONS AND SEQUENCE LISTING STATEMENT

This application claims priority from U.S. provisional application 62/760,649 filed on Nov. 13, 2019 and herewith incorporated in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 580127_424USPC_SEQUENCE_LISTING.txt. The text file is 293 KB, was created on Apr. 27, 2021, and is being submitted electronically via EFS-Web.

### TECHNOLOGICAL FIELD

The present disclosure concerns a recombinant yeast host cell capable modified to express an heterologous trehalase and increase trehalose production during fermentation.

### BACKGROUND

Whereas glucoamylase and alpha-amylase reduction represent a substantial cost savings for ethanol producers, increasing overall yield is significantly more valuable. One potential for yield improvements is targeting of residual fermentable sugars. For example, a typical corn ethanol fermentation will have approximately 4 g/L of residual DP2 sugars, comprised of maltose, isolmaltose and the majority being trehalose. These disaccharides represent a potential of an additional 4 g/L ethanol. Trehalose is an essential product of yeast metabolism, typically produced as a stress protectant and carbohydrate reserve. Being a yeast-produced sugar, there is potential for both metabolic engineering strategies to reduce production and/or secretion of trehalases that can hydrolyze the trehalose into two glucose moieties.

Trehalose is a non-reducing disaccharide composed of two glucose molecules linked at the 1-carbon, forming an a-a bond. In yeast, trehalose can act as carbohydrate storage, but more importantly, it has been well characterized to act as a stress protectant against desiccation, high temperatures, ethanol toxicity, and acidic conditions by stabilizing biological membranes and native polypeptides. Intracellular trehalose is well-regulated in yeasts based on an equilibrium between synthesis and degradation. As shown on FIG. 1, in yeasts, trehalose is catalyzed by combining a uridine-diphosphate-glucose moiety to a glucose-6-phosphate to form trehalose-6-phosphate (step 010). The phosphate group is then removed to form trehalose (step 020). The primary pathway (steps 010 and 020) is facilitated by a polypeptide complex encoded by 4 genes: the trehalose-6-phosphate synthase (TPS1), trehalose-6-phosphate phosphatase (TPS2) and two regulatory polypeptides, TPS3 and TSL1. Trehalose can be catabolized into two glucose molecules by either the cytoplasmic trehalase enzyme, NTH1, or the tethered, extracellular trehalase, ATH1 (step 030). The trehalose biosynthetic pathway has also been proposed to be a primary regulator of glycolysis by creating a futile cycle. As glucose is phosphorylated by hexokinase (HXK, step 040), the intracellular free organic phosphate levels are quickly depleted which is required for downstream processes and other metabolic processes. Conversion of glucose-6-phosphate into trehalose not only removes the sugar from glycolysis, creating a buffer, but the pathway also regenerates inorganic phosphate. Another primary control of glycolysis is the inhibition of HXK2 by trehalose-6-phosphate, thereby further slowing the glycolysis flux.

Numerous manipulations of the trehalose pathway in Saccharomyces cerevisiae have been described. Attempts at trehalose manipulations as a means of targeting ethanol yield increase have primarily focused on over-expression of the pathway, particularly with TPS1/TPS2 (Cao et al., 2014; Guo et al., 2011; An et al., 2011). Ge et al. (2013) successfully improved ethanol yields on pure glucose with the over-expression of the TSL1 component, which has also been implicated in glucose signaling. However, deletion of the biosynthetic pathway has proved more challenging. As reviewed by Thevelein and Hohmann (1995), attempts to remove the TPS1 function have led to the decreased ability to grow on readily fermentable carbon sources due to the aforementioned control of glycolysis. Functional analysis of the TPS complex has been extensively studied using knockout approaches (Bell et al., 1998).

It would be highly desirable to be provided with a recombinant host cell capable of improving fermentation yield and which also retain its robustness during fermentation, especially in the presence of a stressor.

### BRIEF SUMMARY

The present disclosure concerns a recombinant robust yeast host cell capable of maintaining fermentation yields during a stressful fermentation as well as processes using the recombinant robust yeast host cell to make a fermentation product from a biomass.

According to a first aspect, the recombinant yeast host cell has a first genetic modification for expressing an heterologous trehalase, and a second genetic modification for increasing trehalose production. In an embodiment, the heterologous trehalase can be a cell-associated trehalase. In another embodiment, the heterolgous trehalase can be a secreted trehalase. In yet a further embodiment, the heterologous trehalase: (a) has the amino acid sequence of SEQ ID NO.: 2, 4, 6, 8, 10, 12, 14, 16, 18, 20, 22, 24, 26, 28, 30, 32, 34, 36 or 38; (b) is a variant of the amino acid sequence of (a) exhibiting trehalase activity; or (c) is a fragment of the amino acid sequence of (a) or (b) exhibiting trehalase activity. In an embodiment, the heterologous trehalase is from Achlya sp., for example Achlya hypogyna, and can have the amino acid sequence of SEQ ID NO: 36, be a variant of the amino acid sequence of SEQ ID NO: 36 exhibiting trehalase activity or be a fragment of the amino acid sequence of SEQ ID NO: 36 or the variant and exhibiting trehalase activity. In another embodiment, the heterologous trehalase is from Ashbya sp., for example Ashbya gossypii and can have the amino acid sequence of SEQ ID NO: 24, be a variant of the amino acid sequence of SEQ ID NO: 24 exhibiting trehalase activity or be a fragment of the amino acid sequence of SEQ ID NO: 24 or the variant and exhibiting trehalase activity. In yet another embodiment, the heterologous trehalase is from Aspergillus sp. In such embodiment, the trehalase can be from Aspergillus clavatus, and have, for example, the amino acid sequence of SEQ ID NO: 14, be a variant of the amino acid sequence of SEQ ID NO: 14 exhibiting trehalase activity or be a fragment of the amino acid sequence of SEQ ID NO: 14 or the variant and exhibiting trehalase activity. In such embodiment, the heterologous trehalase is from Aspergillus flavus, and can have the amino acid sequence of SEQ ID NO: 6, be a variant of the amino acid sequence of SEQ ID NO: 6 exhibiting trehalase activity or be a fragment of the amino acid sequence of SEQ ID NO: 6 or the variant and exhibiting trehalase activity. Still in such embodiment, the heterologous trehalase is from Aspergillus fumigatus, and have, for example, the amino acid sequence of SEQ ID NO: 2, be a variant of the amino acid sequence of SEQ ID NO: 2 exhibiting trehalase activity or be a fragment of the amino acid sequence of SEQ ID NO: 2 or the variant and exhibiting trehalase activity. Still yet in this embodiment, the heterologous trehalase is from Aspergillus lentulus, and can have the amino acid sequence of SEQ ID NO: 30, be a variant of the amino acid sequence of SEQ ID NO: 30 exhibiting trehalase activity or be a fragment of the amino acid sequence of SEQ ID NO: 30 or the variant and exhibiting trehalase activity. Still further in this embodiment, the heterologous trehalase is from Aspergillus ochraceoroseus, and can have the amino acid sequence of SEQ ID NO: 32, be a variant of the amino acid sequence of SEQ ID NO: 32 exhibiting trehalase activity or be a fragment of the amino acid sequence of SEQ ID NO: 32 or the variant and exhibiting trehalase activity. In yet another embodiment, the heterologous trehalase is from Escovopsis sp., for example from Escovopsis weberi, and can have the amino acid sequence of SEQ ID NO: 10, be a variant of the amino acid sequence of SEQ ID NO: 10 exhibiting trehalase activity or be a fragment of the amino acid sequence of SEQ ID NO: 10 or the variant and exhibiting trehalase activity. In still another embodiment, he heterologous trehalase is from Fusarium sp., for example from Fusarium oxysporum, and can have the amino acid sequence of SEQ ID NO: 8, be a variant of the amino acid sequence of SEQ ID NO: 8 exhibiting trehalase activity or be a fragment of the amino acid sequence of SEQ ID NO: 8 or the variant and exhibiting trehalase activity. In yet another embodiment, the heterologous trehalase is from Kluyveromyces sp., for example from Kluyveromyces marxianus, and can have the amino acid sequence of SEQ ID NO: 20, bea variant of the amino acid sequence of SEQ ID NO: 20 exhibiting trehalase activity or be a fragment of the amino acid sequence of SEQ ID NO: 20 or the variant and exhibiting trehalase activity. In still another embodiment, the heterologous trehalase is from Komagataella sp., for example from Komagataella phaffii, and can have the amino acid sequence of SEQ ID NO: 22, be a variant of the amino acid sequence of SEQ ID NO: 22 exhibiting trehalase activity or be a fragment of the amino acid sequence of SEQ ID NO: 22 or the variant and exhibiting trehalase activity. In yet a further embodiment, the heterologous trehalase is from Metarhizium sp., for example from Metarhizium anisopliae, and can have the amino acid sequence of SEQ ID NO: 16, be a variant of the amino acid sequence of SEQ ID NO: 16 exhibiting trehalase activity or be a fragment of the amino acid sequence of SEQ ID NO: 16 or the variant and exhibiting trehalase activity. In still another embodiment, the heterologous trehalase is from Microsporum sp., for example from Microsporum gypseum, and can have the amino acid sequence of SEQ ID NO: 12, be a variant of the amino acid sequence of SEQ ID NO: 12 exhibiting trehalase activity or be a fragment of the amino acid sequence of SEQ ID NO: 12 or the variant and exhibiting trehalase activity. In yet a further embodiment, the heterologous trehalase is from Neosartorya sp., for example from Neosartorya udagawae, and can have the amino acid sequence of SEQ ID NO: 4, be a variant of the amino acid sequence of SEQ ID NO: 4 exhibiting trehalase activity or be a fragment of the amino acid sequence of SEQ ID NO: 4 or the variant and exhibiting trehalase activity. In a further embodiment, the heterologous trehalase is from Neurospora sp., for example from Neurospora crassa, and can have the amino acid sequence of SEQ ID NO: 26, be a variant of the amino acid sequence of SEQ ID NO: 26 exhibiting trehalase activity or be a fragment of the amino acid sequence of SEQ ID NO: 26 or the variant and exhibiting trehalase activity. In still another embodiment, the heterologous trehalase is from Ogataea sp., for example from Ogataea parapolymorpha, and can have the amino acid sequence of SEQ ID NO: 18, be a variant of the amino acid sequence of SEQ ID NO: 18 exhibiting trehalase activity or be a fragment of the amino acid sequence of SEQ ID NO: 18 or the variant and exhibiting trehalase activity. In another embodiment, the heterologous trehalase is from Rhizoctonia sp., for example from Rhizoctonia solani, and can have the amino acid sequence of SEQ ID NO: 34, be a variant of the amino acid sequence of SEQ ID NO: 34 exhibiting trehalase activity or be a fragment of the amino acid sequence of SEQ ID NO: 34 or the variant and exhibiting trehalase activity. In still a further embodiment, the heterologous trehalase is from Schizopora sp., for example from Schizopora paradoxa, and can have the amino acid sequence of SEQ ID NO: 38, be a variant of the amino acid sequence of SEQ ID NO: 38 exhibiting trehalase activity or be a fragment of the amino acid sequence of SEQ ID NO: 38 or the variant and exhibiting trehalase activity. In a further embodiment, the heterologous trehalase is from Thielavia sp., for example from Thielavia terrestris, and can have the amino acid sequence of SEQ ID NO: 28, be a variant of the amino acid sequence of SEQ ID NO: 28 exhibiting trehalase activity or be a fragment of the amino acid sequence of SEQ ID NO: 28 or the variant and exhibiting trehalase activity. In yet another embodiment, the second genetic modification allows the expression of a second (heterologous) enzyme involved in producing trehalose (TPS1 and/or TPS2 for example) and/or a second (heterologous) regulatory polypeptide involved in regulating trehalose production (TPS3 and/or TSL1 for example). In still another embodiment, the recombinant yeast host cell overexpresses the second enzyme and/or the second regulatory polypeptide. In an embodiment, the second genetic modification allows the expression of at least one of TPS1, TPS2, TPS3 or TSL1. In another embodiment, the second genetic modification allows the expression of TPS1. In a further embodiment, the second genetic modification allows the expression of TPS2. In still another embodiment, the second genetic modification allows the expression of TPS3. In yet another embodiment, the second genetic modification allows the expression of TSL1. In some embodiments, the recombinant yeast host cell exhibits increased robustness in the presence of a stressor, when compared to a corresponding recombinant yeast host cell having the first genetic modification and lacking the second genetic modification. In some additional embodiment, the recombinant yeast host cell further comprises at least one of: a third genetic modification allowing or increasing the expression of an heterologous saccharolytic enzyme; a fourth genetic modification allowing or increasing the production of formate; a fifth genetic modification allowing or increasing the utilization of acetyl-CoA; a sixth genetic modification limiting the production of glycerol; and/or a seventh genetic modification facilitating the transport of glycerol in the recombinant yeast host cell. In some embodiments, the recombinant yeast host cell is from the genus Saccharomyces sp., for example Saccharomyces cerevisiae.

In a second aspect, the present disclosure provides a process for converting a biomass into a fermentation product, the process comprises contacting the biomass with the recombinant yeast host cell defined herein under conditions to allow the conversion of at least a part of the biomass into the fermentation product. In some embodiments, the biomass comprises corn which can optionally be provided as a mash. In some additional embodiments, the fermentation product is an alcohol, such as ethanol. In yet another embodiment, the process is conducted, at least in part, in the presence of a stressor.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 (prior art) illustrates the trehalose synthesis pathway. Abbreviations: HXK=hexokinase; GLK=glucokinase; PGM=Phosphoglucomutase; UGP1=UDP-glucose pyrophosphorylase; GSY=glycogen synthase; GPH=Glycogen phosphorylase; TPS1=Trehalose-6-Phosphate Synthase; TPS3=Trehalose-6-Phosphate Synthase; TSL1=Trehalose Synthase Long chain; TPS2=Trehalose-6-phosphate Phosphatase; NTH=Neutral Trehalase; ATH1=Acid trehalase.

FIG. 2 provides the average secreted trehalase activity (as measured with the DNS assay) of ten (10) clonal isolates for each enzyme candidate compared to the MP244 trehalase. All strains tested were derived from the M2390 background. The tested strains are identified using the nomenclature of the trehalase expressed. Results are shown as the absorbance at 540 nm in function of trehalase expressed.

FIG. 3 provide a time course of trehalase activity for the top five candidates tested. Results are shown as the absorbance at 540 nm in function of trehalase expressed for the different time points (30 minutes=white bars, 60 minutes=light grey bars, 90 minutes=dark grey bars).

FIG. 4 shows the effect of expressing different heterologous trehalase on the ethanol yield and glucose consumption in a permissive fermentation. The expression of the N. crassa trehalase (MP1067) in strain M16283 increased ethanol yield by ˜0.5%. The fermentations were conducted at permissive temperatures. Bars represent ethanol yield (in g/L) at 50 h (left axis). Squares represent glucose content (in g/L) at 50 h (right axis).

FIG. 5 shows the effect of expressing different heterologous trehalase on the ethanol yield and glucose consumption in a stress (high temperatures) fermentation. The expression of N. crassa trehalase (MP1067) in strain M16283 did not lose robustness when exposed to high temperature fermentation. Bars represent ethanol yield (in g/L) at 50 h (left axis). Lozenges represent glucose content (in g/L) at 50 h (right axis).

FIG. 6A to 6C show the results of fermentation of the strains overexpressing trehalase/TSL1 or of control strains. (FIG. 6A) Results are shown for the permissive fermentations as the amount of ethanol (bars, g/L, left axis) and of glycerol (♦, g/L, right axis) produced. (FIG. 6B) Results are shown for the lactic fermentations as the amount of ethanol (bars, g/L, left axis) and glycerol (♦, g/L, right axis) after 50 h as well as the amount of residual glucose (▴, g/L. right axis). (FIG. 6C) Results are shown for the permissive, high temperature stress fermentations and bacterial stress fermentations as the amount of ethanol (bars, g/L, left axis) and of glycerol (♦, g/L, right axis) produced after 50 h as well as the amount of residual glucose (▪, g/L. right axis).

FIG. 7 illustrates the reduction in trehalose measured at the end of fermentation for strains engineered to express a recombinant trehalase. Supernatants obtained at the end of fermentation (permissive=black bars, bacterial stress=grey bars) were run on the Dionex and measured for residual trehalose. Strain overexpressing a trehalase together with TSL1 (M16750 and M16573) showed a reduction in trehalose compared to the parental control strain M15419. Results are shown as the trehalose content in the supernatant (in g/L) in function of the strain tested and the type of fermentation conducted.

FIG. 8 shows the counting of live and dead cells at the end of a permissive fermentation. Results are shown as the number of live (black bars), dead (light gray bars) and total (dark gray bars) yeasts in function of the strain tested.

### DETAILED DESCRIPTION

In accordance with the present disclosure, there is provided a recombinant yeast host cell having an increased ability to degrade trehalose (preferably outside the cell) to increase fermentation yield and an increased ability to synthesize trehalose (preferably inside the cell) to improve fermentation yield and maintain the robustness of the cell during fermentation. Expressing an heterologous trehalase (and in some embodiments, an heterologous trehalase exhibiting its activity mainly outside the recombinant yeast host cell) in a recombinant host cell has the potential to increase fermentation yield (especially alcohol yield) as it provides the cell with the possibility of using trehalose as a carbon source during fermentation. However, as shown in the Examples below and discussed herein, attempts at expressing an heterologous trehalase have cause a reduction in the robustness of the recombinant yeast host cell during fermentation, especially in the presence of a stressor. Unexpectedly, the introduction of a second genetic modification in the recombinant yeast host cell allowing an increase trehalose production restored the robustness in the recombinant yeast host cell and allowed achieving increased fermentation yield.

Recombinant Yeast Host Cell

The present disclosure concerns recombinant yeast host cells. The recombinant yeast host cell are obtained by introducing at least two distinct genetic modifications in a corresponding ancestral or native yeast host cell. The genetic modifications in the recombinant yeast host cell of the present disclosure comprise, consist essentially of or consist of a first genetic modification for expressing an heterologous trehalase and a second genetic modification for increasing trehalose production. In the context of the present disclosure, the expression “the genetic modifications in the recombinant yeast host consist essentially of a first genetic modification and a second genetic modification” refers to the fact that the recombinant yeast host cell can include other genetic modifications which are unrelated or not directly related to the anabolism or the catabolism of trehalose.

When the genetic modification is aimed at reducing or inhibiting the expression of a specific targeted gene (which is endogenous to the host cell), the genetic modifications can be made in one or both copies of the targeted gene(s). When the genetic modification is aimed at increasing the expression of a specific targeted gene, the genetic modification can be made in one or multiple genetic locations. In the context of the present disclosure, when recombinant yeast host cells are qualified as being “genetically engineered”, it is understood to mean that they have been manipulated to either add at least one or more heterologous or exogenous nucleic acid residue and/or remove at least one endogenous (or native) nucleic acid residue. In some embodiments, the one or more nucleic acid residues that are added can be derived from an heterologous cell or the recombinant yeast host cell itself. In the latter scenario, the nucleic acid residue(s) is (are) added at a genomic location which is different than the native genomic location. The genetic manipulations did not occur in nature and are the results of in vitro manipulations of the native yeast or bacterial host cell.

When expressed in a recombinant yeast host cell, the polypeptides (including the enzymes) described herein are encoded on one or more heterologous nucleic acid molecule. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that is removed from the source organism and subsequently reintroduced into the source organism in a form that is different from the corresponding native gene, e.g., not in its natural location in the organism's genome. The heterologous nucleic acid molecule is purposively introduced into the recombinant host cell. The term “heterologous” as used herein also refers to an element (nucleic acid or polypeptide) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

When an heterologous nucleic acid molecule is present in the recombinant yeast host cell, it can be integrated in the yeast host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the host cell's genome. In such embodiment, the nucleic acid molecule can be stable and self-replicating.

In some embodiments, heterologous nucleic acid molecules which can be introduced into the recombinant yeast host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure comprise a coding region for the one or more polypeptides (including enzymes) to be expressed by the recombinant host cell. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing sites, effector binding sites and stem-loop structures. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise a non-coding region, for example a transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced and optionally maintained in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a host cell.

In the heterologous nucleic acid molecule described herein, the promoter and the nucleic acid molecule coding for the one or more polypeptides (including enzymes) can be operatively linked to one another. In the context of the present disclosure, the expressions “operatively linked” or “operatively associated” refers to fact that the promoter is physically associated to the nucleotide acid molecule coding for the one or more enzyme in a manner that allows, under certain conditions, for expression of the one or more enzyme from the nucleic acid molecule. In an embodiment, the promoter can be located upstream (5′) of the nucleic acid sequence coding for the one or more enzyme. In still another embodiment, the promoter can be located downstream (3′) of the nucleic acid sequence coding for the one or more enzyme. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the one or more enzyme. The promoters can be located, in view of the nucleic acid molecule coding for the one or more polypeptide, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as polypeptide binding domains (consensus sequences) responsible for the binding of the polymerase.

The promoter can be heterologous to the nucleic acid molecule encoding the one or more polypeptides. The promoter can be heterologous or derived from a strain being from the same genus or species as the recombinant yeast host cell. In an embodiment, the promoter is derived from the same genus or species of the yeast host cell and the heterologous polypeptide is derived from different genus that the host cell. In an embodiment, the promoter used in the heterologous nucleic acid molecule is the same promoter that controls the expression of the encoded polypeptide in its native context.

In an embodiment, the present disclosure concerns the expression of one or more polypeptide (including an enzyme), a variant thereof or a fragment thereof in a recombinant host cell. A variant comprises at least one amino acid difference when compared to the amino acid sequence of the native polypeptide (enzyme) and exhibits a biological activity substantially similar to the native polypeptide. The polypeptide/enzyme “variants” have at least 50%, 55%, 60%, 65%, 70%, 75%, 80%, 85%, 90%, 95%, 96%, 97%, 98% or 99% identity to the polypeptide described herein. The heterologous trehalase “variants” can have at least 50%, 55%, 60%, 65%, 70%, 75%, 80%, 85%, 90%, 95%, 96%, 97%, 98% or 99% biological activity when compared to the native polypeptide. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the polypeptide can be a conservative variant or an allelic variant. As used herein, a conservative variant refers to alterations in the amino acid sequence that do not adversely affect the biological functions of the polypeptide/enzyme. A substitution, insertion or deletion is said to adversely affect the polypeptide when the altered sequence prevents or disrupts a biological function associated with the enzyme. For example, the overall charge, structure or hydrophobic-hydrophilic properties of the polypeptide can be altered without adversely affecting a biological activity. Accordingly, the amino acid sequence can be altered, for example to render the polypeptide more hydrophobic or hydrophilic, without adversely affecting the biological activities of the enzyme.

The polypeptide can be a fragment of the polypeptide or fragment of the variant polypeptide. A polypeptide fragment comprises at least one less amino acid residue when compared to the amino acid sequence of the possesses and still possess a biological activity substantially similar to the native full-length polypeptide or polypeptide variant. Polypeptide “fragments” have at least at least 100, 200, 300, 400, 500 or more consecutive amino acids of the polypeptide or the polypeptide variant. The heterologous trehalase “fragments” can have at least 50%, 55%, 60%, 65%, 70%, 75%, 80%, 85%, 90%, 95%, 96%, 97%, 98% or 99% identity to the polypeptide or the variant polypeptide. The heterologous trehalase “fragments” can have at least 50%, 55%, 60%, 65%, 70%, 75%, 80%, 85%, 90%, 95%, 96%, 97%, 98% or 99% biological activity when compared to the native polypeptide or the variant polypeptide. In some embodiments, fragments of the polypeptides can be employed for producing the corresponding full-length enzyme by peptide synthesis. Therefore, the fragments can be employed as intermediates for producing the full-length polypeptides.

In some additional embodiments, the present disclosure also provides expressing a polypeptide encoded by a gene ortholog of a gene known to encode the polypeptide. A “gene ortholog” is understood to be a gene in a different species that evolved from a common ancestral gene by speciation. In the context of the present disclosure, a gene ortholog encodes polypeptide exhibiting a biological activity substantially similar to the native polypeptide.

In some further embodiments, the present disclosure also provides expressing a polypeptide encoded by a gene paralog of a gene known to encode the polypeptide. A “gene paralog” is understood to be a gene related by duplication within the genome. In the context of the present disclosure, a gene paralog encodes a polypeptide that could exhibit additional biological functions when compared to the native polypeptide.

In the context of the present disclosure, the recombinant/native host cell is a yeast. Suitable yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the host cell can be an oleaginous microalgae host cell (e.g., for example, from the genus Thraustochytrium or Schizochytrium). In an embodiment, the recombinant yeast host cell is from the genus Saccharomyces and, in some additional embodiments, from the species Saccharomyces cerevisiae.

Since the recombinant yeast host cell can be used for the fermentation of a biomass and the generation of fermentation product, it is contemplated herein that it has the ability to convert a biomass into a fermentation product without including the additional genetic modifications described herein. In an embodiment, the recombinant yeast host cell has the ability to convert starch into ethanol during fermentation, as it is described below. In still another embodiment, the recombinant yeast host cell of the present disclosure can be genetically modified to provide or increase the biological activity of one or more polypeptide involved in the fermentation of the biomass and the generation of the fermentation product.

First genetic modification: expression of an heterologous trehalase

The introduction of the first genetic modification in the recombinant yeast host cell confers an increased trehalase activity to the recombinant yeast host cell. Preferably, the increased trehalase activity is observed mainly outside the recombinant yeast host cell, even though it is originally synthesized inside the recombinant yeast host cell. The first genetic modification can be introducing a first heterologous nucleic acid molecule encoding the heterologous trehalase in the recombinant yeast host cell. This first genetic modification can provide a recombinant yeast host cell having a first heterologous nucleic acid molecule encoding the heterologous trehalase.

Trehalases are glycoside hydrolases capable of converting trehalose into glucose. Trehalases have been classified under EC number 3.2.1.28. Trehalases can be classified into two broad categories based on their optimal pH: neutral trehalases (having an optimum pH of about 7) and acid trehalases (having an optimum pH of about 4.5). The heterologous trehalases that can be used in the context of the present disclosure can be of various origins such as bacterial, fungal or plant origin. In a specific embodiment, the trehalase is from fungal origin. In an embodiment, the trehalase is from Aspergillus sp., for example Aspergillus fumigatus which can have, in some embodiments, the amino acid sequence of SEQ ID NO: 2, be a variant of the amino acid sequence of SEQ ID NO: 2 or be a fragment of the amino acid sequence of SEQ ID NO: 2. In such embodiment, the trehalase can be encoded, for example, by the nucleic acid sequence of SEQ ID NO: 1. In an embodiment, the trehalase is from Neosartorya sp., for example Neosartorya udagawae which can have, in some embodiments, the amino acid sequence of SEQ ID NO: 4, be a variant of the amino acid sequence of SEQ ID NO: 4 or be a fragment of the amino acid sequence of SEQ ID NO: 4. In such embodiment, the trehalase can be encoded, for example, by the nucleic acid sequence of SEQ ID NO: 3. In an embodiment, the trehalase is from Aspergillus sp., for example Aspergillus flavus which can have, in some embodiments, the amino acid sequence of SEQ ID NO: 6, be a variant of the amino acid sequence of SEQ ID NO: 6 or be a fragment of the amino acid sequence of SEQ ID NO: 6. In such embodiment, the trehalase can be encoded, for example, by the nucleic acid sequence of SEQ ID NO: 5. In an embodiment, the trehalase is from Fusarium sp., for example Fusarium oxysporum which can have, in some embodiments, the amino acid sequence of SEQ ID NO: 8, be a variant of the amino acid sequence of SEQ ID NO: 8 or be a fragment of the amino acid sequence of SEQ ID NO: 8. In such embodiment, the trehalase can be encoded, for example, by the nucleic acid sequence of SEQ ID NO: 7. In an embodiment, the trehalase is from Escovopsis sp., for example Escovopsis weberi which can have, in some embodiments, the amino acid sequence of SEQ ID NO: 10, be a variant of the amino acid sequence of SEQ ID NO: 10 or be a fragment of the amino acid sequence of SEQ ID NO: 10. In such embodiment, the trehalase can be encoded, for example, by the nucleic acid sequence of SEQ ID NO: 9. In an embodiment, the trehalase is from Microsporum sp., for example Microsporum gypseum which can have, in some embodiments, the amino acid sequence of SEQ ID NO: 12, be a variant of the amino acid sequence of SEQ ID NO: 12 or be a fragment of the amino acid sequence of SEQ ID NO: 12. In such embodiment, the trehalase can be encoded, for example, by the nucleic acid sequence of SEQ ID NO: 11. In an embodiment, the trehalase is from Aspergillus sp., for example Aspergillus clavatus which can have, in some embodiments, the amino acid sequence of SEQ ID NO: 14, be a variant of the amino acid sequence of SEQ ID NO: 14 or be a fragment of the amino acid sequence of SEQ ID NO: 14. In such embodiment, the trehalase can be encoded, for example, by the nucleic acid sequence of SEQ ID NO: 13. In an embodiment, the trehalase is from Metarhizium sp., for example Metarhizium anisopliae which can have, in some embodiments, the amino acid sequence of SEQ ID NO: 16, be a variant of the amino acid sequence of SEQ ID NO: 16 or be a fragment of the amino acid sequence of SEQ ID NO: 16. In such embodiment, the trehalase can be encoded, for example, by the nucleic acid sequence of SEQ ID NO: 15. In an embodiment, the trehalase is from Ogataea sp., for example Ogataea parapolymorpha which can have, in some embodiments, the amino acid sequence of SEQ ID NO: 18, be a variant of the amino acid sequence of SEQ ID NO: 18 or be a fragment of the amino acid sequence of SEQ ID NO: 18. In such embodiment, the trehalase can be encoded, for example, by the nucleic acid sequence of SEQ ID NO: 17. In an embodiment, the trehalase is from Kluyveromyces sp., for example Kluyveromyces marxianus which can have, in some embodiments, the amino acid sequence of SEQ ID NO: 20, be a variant of the amino acid sequence of SEQ ID NO: 20 or be a fragment of the amino acid sequence of SEQ ID NO: 20. In such embodiment, the trehalase can be encoded, for example, by the nucleic acid sequence of SEQ ID NO: 19. In an embodiment, the trehalase is from Komagataella sp., for example Komagataella phaffii which can have, in some embodiments, the amino acid sequence of SEQ ID NO: 22, be a variant of the amino acid sequence of SEQ ID NO: 22 or be a fragment of the amino acid sequence of SEQ ID NO: 22. In such embodiment, the trehalase can be encoded, for example, by the nucleic acid sequence of SEQ ID NO: 21. In an embodiment, the trehalase is from Ashbya sp., for example Ashbya gossypii which can have, in some embodiments, the amino acid sequence of SEQ ID NO: 24, be a variant of the amino acid sequence of SEQ ID NO: 24 or be a fragment of the amino acid sequence of SEQ ID NO: 24. In such embodiment, the trehalase can be encoded, for example, by the nucleic acid sequence of SEQ ID NO: 23. In an embodiment, the trehalase is from Neurospora sp., for example Neurospora crassa which can have, in some embodiments, the amino acid sequence of SEQ ID NO: 26, be a variant of the amino acid sequence of SEQ ID NO: 26 or be a fragment of the amino acid sequence of SEQ ID NO: 26. In such embodiment, the trehalase can be encoded, for example, by the nucleic acid sequence of SEQ ID NO: 25. In an embodiment, the trehalase is from Thielavia sp., for example Thielavia terrestris which can have, in some embodiments, the amino acid sequence of SEQ ID NO: 28, be a variant of the amino acid sequence of SEQ ID NO: 28 or be a fragment of the amino acid sequence of SEQ ID NO: 28. In such embodiment, the trehalase can be encoded, for example, by the nucleic acid sequence of SEQ ID NO: 27. In an embodiment, the trehalase is from Aspergillus sp., for example Aspergillus lentulus which can have, in some embodiments, the amino acid sequence of SEQ ID NO: 30, be a variant of the amino acid sequence of SEQ ID NO: 30 or be a fragment of the amino acid sequence of SEQ ID NO: 30. In such embodiment, the trehalase can be encoded, for example, by the nucleic acid sequence of SEQ ID NO: 29. In an embodiment, the trehalase is from Aspergillus sp., for example Aspergillus ochraceoroseus which can have, in some embodiments, the amino acid sequence of SEQ ID NO: 32, be a variant of the amino acid sequence of SEQ ID NO: 32 or be a fragment of the amino acid sequence of SEQ ID NO: 32. In such embodiment, the trehalase can be encoded, for example, by the nucleic acid sequence of SEQ ID NO: 31. In an embodiment, the trehalase is from Rhizoctonia sp., for example Rhizoctonia solani which can have, in some embodiments, the amino acid sequence of SEQ ID NO: 34, be a variant of the amino acid sequence of SEQ ID NO: 34 or be a fragment of the amino acid sequence of SEQ ID NO: 34. In such embodiment, the trehalase can be encoded, for example, by the nucleic acid sequence of SEQ ID NO: 33. In an embodiment, the trehalase is from Achlya sp., for example Achlya hypogyna which can have, in some embodiments, the amino acid sequence of SEQ ID NO: 36, be a variant of the amino acid sequence of SEQ ID NO: 36 or be a fragment of the amino acid sequence of SEQ ID NO: 36. In such embodiment, the trehalase can be encoded, for example, by the nucleic acid sequence of SEQ ID NO: 35. In an embodiment, the trehalase is from Schizopora sp., for example Schizopora paradoxa which can have, in some embodiments, the amino acid sequence of SEQ ID NO: 38, be a variant of the amino acid sequence of SEQ ID NO: 38 or be a fragment of the amino acid sequence of SEQ ID NO: 38. In such embodiment, the trehalase can be encoded, for example, by the nucleic acid sequence of SEQ ID NO: 38. In a specific embodiment, the heterologous trehalase has the amino acid sequence of SEQ ID NO: 2, 4, 20, 24, 26, 28, 30 or 36, is a variant of the amino acid sequence of SEQ ID NO: 2, 4, 20, 24, 26, 28, 30 of 36 or is a fragment of the amino acid sequence of SEQ ID NO: 2, 4, 20, 24, 26, 28, 30 of 36. In an embodiment, the heterologous trehalase has the amino acid sequence of SEQ ID NO: 2 or 4, is a variant of the amino acid sequence of SEQ ID NO: 2 or 4 or is a fragment of the amino acid sequence NO: 2 or 4. In an embodiment, the heterologous trehalase has the amino acid sequence of SEQ ID NO: 2 or 20, is a variant of the amino acid sequence of SEQ ID NO: 2 or 20 or is a fragment of the amino acid sequence NO: 2 or 20. In an embodiment, the heterologous trehalase has the amino acid sequence of SEQ ID NO: 2 or 24, is a variant of the amino acid sequence of SEQ ID NO: 2 or 24 or is a fragment of the amino acid sequence NO: 2 or 24. In an embodiment, the heterologous trehalase has the amino acid sequence of SEQ ID NO: 2 or 26, is a variant of the amino acid sequence of SEQ ID NO: 2 or 26 or is a fragment of the amino acid sequence NO: 2 or 26. In an embodiment, the heterologous trehalase has the amino acid sequence of SEQ ID NO: 2 or 28, is a variant of the amino acid sequence of SEQ ID NO: 2 or 28 or is a fragment of the amino acid sequence NO: 2 or 28. In an embodiment, the heterologous trehalase has the amino acid sequence of SEQ ID NO: 2 or 30, is a variant of the amino acid sequence of SEQ ID NO: 2 or 30 or is a fragment of the amino acid sequence NO: 2 or 30. In an embodiment, the heterologous trehalase has the amino acid sequence of SEQ ID NO: 2 or 36, is a variant of the amino acid sequence of SEQ ID NO: 2 or 36 or is a fragment of the amino acid sequence NO: 2 or 36. In an embodiment, the heterologous trehalase has the amino acid sequence of SEQ ID NO: 4 or 20, is a variant of the amino acid sequence of SEQ ID NO: 4 or 20 or is a fragment of the amino acid sequence NO: 4 or 20. In an embodiment, the heterologous trehalase has the amino acid sequence of SEQ ID NO: 4 or 24, is a variant of the amino acid sequence of SEQ ID NO: 4 or 24 or is a fragment of the amino acid sequence NO: 4 or 24. In an embodiment, the heterologous trehalase has the amino acid sequence of SEQ ID NO: 4 or 26, is a variant of the amino acid sequence of SEQ ID NO: 4 or 26 or is a fragment of the amino acid sequence NO: 4 or 26. In an embodiment, the heterologous trehalase has the amino acid sequence of SEQ ID NO: 4 or 28, is a variant of the amino acid sequence of SEQ ID NO: 4 or 28 or is a fragment of the amino acid sequence NO: 4 or 28 . In an embodiment, the heterologous trehalase has the amino acid sequence of SEQ ID NO: 4 or 30, is a variant of the amino acid sequence of SEQ ID NO: 4 or 30 or is a fragment of the amino acid sequence NO: 4 or 30. In an embodiment, the heterologous trehalase has the amino acid sequence of SEQ ID NO: 4 or 36, is a variant of the amino acid sequence of SEQ ID NO: 4 or 36 or is a fragment of the amino acid sequence NO: 4 or 36. In an embodiment, the heterologous trehalase has the amino acid sequence of SEQ ID NO: 20 or 24, is a variant of the amino acid sequence of SEQ ID NO: 20 or 24 or is a fragment of the amino acid sequence NO: 20 or 24. In an embodiment, the heterologous trehalase has the amino acid sequence of SEQ ID NO: 20 or 26, is a variant of the amino acid sequence of SEQ ID NO: 20 or 26 or is a fragment of the amino acid sequence NO: 20 or 26. In an embodiment, the heterologous trehalase has the amino acid sequence of SEQ ID NO: 20 or 28, is a variant of the amino acid sequence of SEQ ID NO: 20 or 28 or is a fragment of the amino acid sequence NO: 20 or 28. In an embodiment, the heterologous trehalase has the amino acid sequence of SEQ ID NO: 20 or 30, is a variant of the amino acid sequence of SEQ ID NO: 20 or 30 or is a fragment of the amino acid sequence NO: 20 or 30. In an embodiment, the heterologous trehalase has the amino acid sequence of SEQ ID NO: 20 or 36, is a variant of the amino acid sequence of SEQ ID NO: 20 or 36 or is a fragment of the amino acid sequence NO: 20 or 36. In an embodiment, the heterologous trehalase has the amino acid sequence of SEQ ID NO: 24 or 26, is a variant of the amino acid sequence of SEQ ID NO: 24 or 26 or is a fragment of the amino acid sequence NO: 24 or 26. In an embodiment, the heterologous trehalase has the amino acid sequence of SEQ ID NO: 24 or 28, is a variant of the amino acid sequence of SEQ ID NO: 24 or 28 or is a fragment of the amino acid sequence NO: 24 or 28. In an embodiment, the heterologous trehalase has the amino acid sequence of SEQ ID NO: 24 or 30, is a variant of the amino acid sequence of SEQ ID NO: 24 or 30 or is a fragment of the amino acid sequence NO: 24 or 30. In an embodiment, the heterologous trehalase has the amino acid sequence of SEQ ID NO: 24 or 36, is a variant of the amino acid sequence of SEQ ID NO: 24 or 36 or is a fragment of the amino acid sequence NO: 24 or 36. In an embodiment, the heterologous trehalase has the amino acid sequence of SEQ ID NO: 26 or 28, is a variant of the amino acid sequence of SEQ ID NO: 26 or 28 or is a fragment of the amino acid sequence NO: 26 or 28. In an embodiment, the heterologous trehalase has the amino acid sequence of SEQ ID NO: 26 or 30, is a variant of the amino acid sequence of SEQ ID NO: 26 or 30 or is a fragment of the amino acid sequence NO: 26 or 30.

In an embodiment, the heterologous trehalase has the amino acid sequence of SEQ ID NO: 26 or 36, is a variant of the amino acid sequence of SEQ ID NO: 26 or 36 or is a fragment of the amino acid sequence NO: 26 or 36. In an embodiment, the heterologous trehalase has the amino acid sequence of SEQ ID NO: 28 or 30, is a variant of the amino acid sequence of SEQ ID NO: 28 or 30 or is a fragment of the amino acid sequence NO: 28 or 30. In an embodiment, the heterologous trehalase has the amino acid sequence of SEQ ID NO: 28 or 36, is a variant of the amino acid sequence of SEQ ID NO: 28 or 36 or is a fragment of the amino acid sequence NO: 28 or 36. In an embodiment, the heterologous trehalase has the amino acid sequence of SEQ ID NO: 30 or 36, is a variant of the amino acid sequence of SEQ ID NO: 30 or 36 or is a fragment of the amino acid sequence NO: 30 or 36. Since the heterologous trehalase is intended to exert its biological activity mainly outside the recombinant yeast host cell, the heterologous trehalase can be selected based on their ability to be translocated outside the cell or alternatively modified to be secreted or remain associated with the external surface of the recombinant yeast host cell membrane.

As indicated above, the present disclosure includes recombinant yeast host cell expressing one or more a variant trehalase. A variant trehalase comprises at least one amino acid difference when compared to the amino acid sequence of the trehalase and exhibits trehalase activity substantially similar to the trehalase. The heterologous “variants” can have at least 50%, 55%, 60%, 65%, 70%, 75%, 80%, 85%, 90%, 95%, 96%, 97%, 98% or 99% identity to the polypeptide having the amino acid sequence of SEQ ID NO: 2, 4, 20, 24, 26, 28, 30 or 36. The heterologous “variants” have at least 50%, 55%, 60%, 65%, 70%, 75%, 80%, 85%, 90%, 95%, 96%, 97%, 98% or 99% identity to the polypeptide having the amino acid sequence of SEQ ID NO: 2. The heterologous “variants” can have at least 50%, 55%, 60%, 65%, 70%, 75%, 80%, 85%, 90%, 95%, 96%, 97%, 98% or 99% identity to the polypeptide having the amino acid sequence of SEQ ID NO: 4. The heterologous “variants” can have at least 50%, 55%, 60%, 65%, 70%, 75%, 80%, 85%, 90%, 95%, 96%, 97%, 98% or 99% identity to the polypeptide having the amino acid sequence of SEQ ID NO: 20. The heterologous “variants” can have at least 50%, 55%, 60%, 65%, 70%, 75%, 80%, 85%, 90%, 95%, 96%, 97%, 98% or 99% identity to the polypeptide having the amino acid sequence of SEQ ID NO: 24. The heterologous “variants” can have at least 50%, 55%, 60%, 65%, 70%, 75%, 80%, 85%, 90%, 95%, 96%, 97%, 98% or 99% identity to the polypeptide having the amino acid sequence of SEQ ID NO: 26. The heterologous “variants” can have at least 50%, 55%, 60%, 65%, 70%, 75%, 80%, 85%, 90%, 95%, 96%, 97%, 98% or 99% identity to the polypeptide having the amino acid sequence of SEQ ID 28. The heterologous “variants” can have at least 50%, 55%, 60%, 65%, 70%, 75%, 80%, 85%, 90%, 95%, 96%, 97%, 98% or 99% identity to the polypeptide having the amino acid sequence of SEQ ID NO: 30. The heterologous “variants” can have at least 50%, 55%, 60%, 65%, 70%, 75%, 80%, 85%, 90%, 95%, 96%, 97%, 98% or 99% identity to the polypeptide having the amino acid sequence of SEQ ID NO: 36. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant trehalase described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the trehalase can be a conservative variant or an allelic variant. As used herein, a conservative variant refers to alterations in the amino acid sequence that do not adversely affect the biological functions of the enzyme. A substitution, insertion or deletion is said to adversely affect the polypeptide when the altered sequence prevents or disrupts a biological function associated with the enzyme. For example, the overall charge, structure or hydrophobic-hydrophilic properties of the polypeptide can be altered without adversely affecting a biological activity. Accordingly, the amino acid sequence can be altered, for example to render the trehalase more hydrophobic or hydrophilic, without adversely affecting the biological activities of the enzyme.

The trehalase can be a fragment of trehalase or fragment of a variant trehalase. A trehalase fragment comprises at least one less amino acid residue when compared to the amino acid sequence of the possesses and still possess a trehalase activity substantially similar to the native full-length polypeptide or polypeptide variant. trehalase “fragments” have at least at least 100, 200, 300, 400, 500 or more consecutive amino acids of the polypeptide or the polypeptide variant. In some embodiments, fragments of the polypeptides can be employed for producing the corresponding full-length enzyme by peptide synthesis. Therefore, the fragments can be employed as intermediates for producing the full-length polypeptides. The heterologous trehalase “fragments” can have at least 50%, 55%, 60%, 65%, 70%, 75%, 80%, 85%, 90%, 95%, 96%, 97%, 98% or 99% identity to the polypeptide having the amino acid sequence of SEQ ID NO: 2, 4, 20, 24, 26, 28, 30 or 36. The heterologous “fragments” have at least 50%, 55%, 60%, 65%, 70%, 75%, 80%, 85%, 90%, 95%, 96%, 97%, 98% or 99% identity to the polypeptide having the amino acid sequence of SEQ ID NO: 2. The heterologous “fragments” can have at least 50%, 55%, 60%, 65%, 70%, 75%, 80%, 85%, 90%, 95%, 96%, 97%, 98% or 99% identity to the polypeptide having the amino acid sequence of SEQ ID NO: 4. The heterologous “fragments” can have at least 50%, 55%, 60%, 65%, 70%, 75%, 80%, 85%, 90%, 95%, 96%, 97%, 98% or 99% identity to the polypeptide having the amino acid sequence of SEQ ID NO: 20. The heterologous “fragments” can have at least 50%, 55%, 60%, 65%, 70%, 75%, 80%, 85%, 90%, 95%, 96%, 97%, 98% or 99% identity to the polypeptide having the amino acid sequence of SEQ ID NO: 24. The heterologous “fragments” can have at least 50%, 55%, 60%, 65%, 70%, 75%, 80%, 85%, 90%, 95%, 96%, 97%, 98% or 99% identity to the polypeptide having the amino acid sequence of SEQ ID NO: 26. The heterologous “fragments” can have at least 50%, 55%, 60%, 65%, 70%, 75%, 80%, 85%, 90%, 95%, 96%, 97%, 98% or 99% identity to the polypeptide having the amino acid sequence of SEQ ID 28. The heterologous “fragments” can have at least 50%, 55%, 60%, 65%, 70%, 75%, 80%, 85%, 90%, 95%, 96%, 97%, 98% or 99% identity to the polypeptide having the amino acid sequence of SEQ ID NO: 30. The heterologous “fragments” can have at least 50%, 55%, 60%, 65%, 70%, 75%, 80%, 85%, 90%, 95%, 96%, 97%, 98% or 99% identity to the polypeptide having the amino acid sequence of SEQ ID NO: 36.

Some heterologous trehalase possess a signal sequence and are presumed to be secreted from the recombinant yeast host cell. For example, the trehalases having the following amino acid sequence do possess a native signal sequence predisposing them to be secreted: SEQ ID NO: 2, 4, 6, 8, 10, 12, 14, 16, 17, 26, 28, 30, 34, 36 and 38. For these heterologous trehalases, it is contemplated to use their native signal sequence or replace it with another signal sequence which will facilitate their secretion from the recombinant yeast host cell. For the other trehalases (those having the amino acid sequence of SEQ ID NO: 18, 20, 22, 24 and 32), it is possible to include an appropriate signal sequence allowing their secretion outside the cell, for example from by including a signal sequence from another trehalase or a signal sequence being recognized as such by the recombinant yeast host cell.

In some embodiments, the secreted heterologous trehalases are released in the culture/fermentation medium and do not remain physically attached to the recombinant yeast cell. In alternative embodiments, the heterologous trehalases of the present disclosure can be secreted, but they remain physically associated with the recombinant yeast host cell. In an embodiment, at least one portion (usually at least one terminus) of the heterologous trehalase is bound, covalently, non-covalently and/or electrostatically for example, to cell wall (and in some embodiments to the cytoplasmic membrane). For example, the heterologous trehalase can be modified to bear one or more transmembrane domains, to have one or more lipid modifications (myristoylation, palmitoylation, farnesylation and/or prenylation), to interact with one or more membrane-associated polypeptide and/or to interactions with the cellular lipid rafts. While the heterologous trehalase may not be directly bound to the cell membrane or cell wall (e.g., such as when binding occurs via a tethering moiety), the polypeptide is nonetheless considered a “cell-associated” heterologous polypeptide according to the present disclosure.

In some embodiments, the heterologous trehalases can be expressed to be located at and associated to the cell wall of the recombinant yeast host cell. In some embodiments, the heterologous polypeptide is expressed to be located at and associated to the external surface of the cell wall of the host cell. Recombinant yeast host cells all have a cell wall (which includes a cytoplasmic membrane) defining the intracellular (e.g., internally-facing the nucleus) and extracellular (e.g., externally-facing) environments. The heterologous trehalase can be located at (and in some embodiments, physically associated to) the external face of the recombinant yeast host's cell wall and, in further embodiments, to the external face of the recombinant yeast host's cytoplasmic membrane. In the context of the present disclosure, the expression “associated to the external face of the cell wall/cytoplasmic membrane of the recombinant yeast host cell” refers to the ability of the heterologous trehalase to physically integrate (in a covalent or non-covalent fashion), at least in part, in the cell wall (and in some embodiments in the cytoplasmic membrane) of the recombinant yeast host cell. The physical integration can be attributed to the presence of, for example, a transmembrane domain on the heterologous polypeptide, a domain capable of interacting with a cytoplasmic membrane polypeptide on the heterologous polypeptide, a post-translational modification made to the heterologous polypeptide (e.g., lipidation), etc.

In some circumstances, it may be warranted to increase or provide cell association to some heterologous trehalases because they exhibit insufficient intrinsic cell association or simply lack intrinsic cell association. In such embodiment, it is possible to provide the heterologous trehalase as a chimeric construct by combining it with a tethering amino acid moiety which will provide or increase attachment to the cell wall of the recombinant yeast host cell. In such embodiment, the chimeric heterologous polypeptide will be considered “tethered”. It is preferred that the amino acid tethering moiety of the chimeric polypeptide be neutral with respect to the biological activity of the heterologous trehalase, e.g., does not interfere with the biological activity (such as, for example, the enzymatic activity) of the heterologous trehalase. In some embodiments, the association of the amino acid tethering moiety with the heterologous polypeptide can increase the biological activity of the heterologous polypeptide (when compared to the non-tethered, “free” form).

In an embodiment, a tethering moiety can be used to be expressed with the heterologous trehalase to locate the heterologous polypeptide to the wall of the recombinant yeast host cell. Various tethering amino acid moieties are known art and can be used in the chimeric polypeptides of the present disclosure. The tethering moiety can be a transmembrane domain found on another polypeptide and allow the chimeric polypeptide to have a transmembrane domain. In such embodiment, the tethering moiety can be derived from the FLO1 polypeptide.

In still another example, the amino acid tethering moiety can be modified post-translation to include a glycosylphosphatidylinositol (GPI) anchor and allow the chimeric polypeptide to have a GPI anchor. GPI anchors are glycolipids attached to the terminus of a polypeptide (and in some embodiments, to the carboxyl terminus of a polypeptide) which allows the anchoring of the polypeptide to the cytoplasmic membrane of the cell membrane. Tethering amino acid moieties capable of providing a GPI anchor include, but are not limited to those associated with/derived from a SED1 polypeptide, a TIR1 polypeptide, a CWP2 polypeptide, a CCW12 polypeptide, a SPI1 polypeptide, a PST1 polypeptide or a combination of a AGA1 and a AGA2 polypeptide. In an embodiment, the tethering moiety provides a GPI anchor and, in still a further embodiment, the tethering moiety is derived from the SPI1 polypeptide or the CCW12 polypeptide.

The tethering amino acid moiety can be a variant of a known/native tethering amino acid moiety. The tethering amino acid moiety can be a fragment of a known/native tethering amino acid moiety or fragment of a variant of a known/native tethering amino acid moiety.

In embodiments in which an amino acid tethering moiety is desirable, the heterologous polypeptide can be provided as a chimeric polypeptide expressed by the recombinant yeast host cell and having one of the following formulae (provided from the amino (NH₂) to the carboxyl (COOH) orientation):

HT-L-TT (I) or

TT-L-HT (II)

In both of these formulae, the residue “HT” refers to the heterologous trehalase moiety, the residue “L” refers to the presence of an optional linker while the residue “TT” refers to an amino acid tethering moiety. In the chimeric polypeptides of formula (I), the amino terminus of the amino acid tether is located (directly or indirectly) at the carboxyl (COOH or C) terminus of the heterologous trehalase moiety. In the chimeric polypeptides of formula (II), the carboxy terminus of the amino acid tether is located (directly or indirectly) at the amino (NH₂ or N) terminus of the heterologous trehalase moiety. Embodiments of chimeric tethered heterologous polypeptides have been disclosed in WO2018/167670 and are included herein in their entirety.

Second Genetic Modification: Increase in Trehalose Production

The introduction of the second genetic modification in the recombinant yeast host cell restores its robustness by increasing trehalose production and more preferably increasing intracellular trehalose levels in the recombinant yeast host cell. In some embodiments, the introduction of the second genetic modification allows for an increase in fermentation yield, such as, for example, an increase in alcoholic yield. The second genetic modification can be introducing a second heterologous nucleic acid molecule encoding one or more polypeptides involved in trehalose production (e.g., a second heterologous enzyme involved in the production of trehalose and/or a second regulatory polypeptide involved in regulating trehalose production) in the recombinant yeast host cell. This second genetic modification can provide a recombinant yeast host cell having a second heterologous nucleic acid molecule encoding one or more polypeptides involved in trehalose production (e.g., a second heterologous enzyme involved in the production of trehalose and/or a second regulatory polypeptide involved in regulating trehalose production).

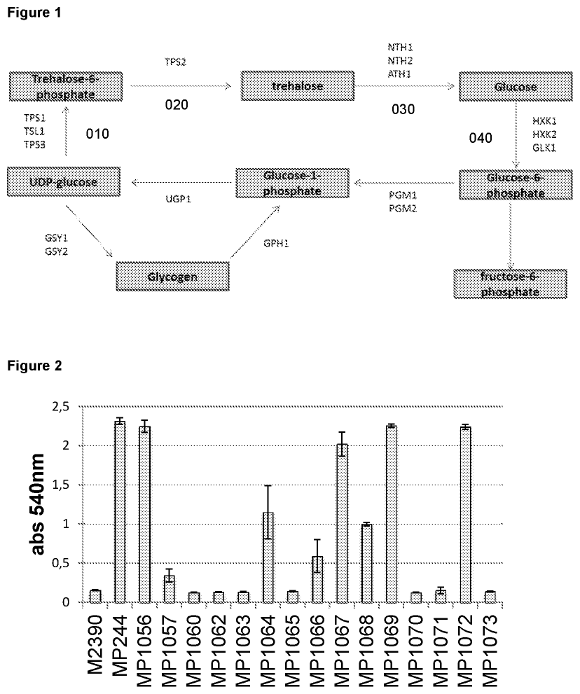

The second genetic modification can be made for allowing the expression of an enzyme involved in the production of trehalose. As indicated on FIG. 1, enzymes involved in trehalose production include, but are not limited to, TPS1, TPS2, HXH1, HXK2, GLK1, PGM1, PGM2 and UGP1 as well as orthologs and paralogs encoding these enzymes. In an embodiment, the second genetic modification in recombinant yeast host cell allows for the expression of at least one of gene encoding for TPS1, TPS2, HXH1, HXK2, GLK1, PGM1, PGM2 or UGP1 including the associated orthologs and paralogs.

In an example, the recombinant yeast host cell can exhibit increased biological activity in at least one of a trehalose-6-phosphate (trehalose-6-P) synthase or a trehalose-6-phosphate phosphatase or both enzymes. As indicated above, this can be done by introducing a strong and/or constitutive promoter to increase the expression of the endogenous trehalose-6-P synthase and/or the endogenous trehalose-6-P phosphatase. Alternatively or in combination, this can also be done by introducing at least one copy of one or more heterologous nucleic acid molecules encoding an heterologous trehalose-6-P synthase and/or an heterologous trehalose-6-P phosphatase. In an embodiment, the recombinant yeast host cell has increased biological activity of a trehalose-6-P synthase, but not of the trehalose-6-P phosphatase. In another embodiment, the recombinant yeast host cell has increased biological activity of a trehalose-6-P phosphatase, but not of the trehalose-6-P synthase. In still another embodiment, the recombinant yeast host cell has increased biological activity in both a trehalose-6-P synthase and a trehalose-6-P phosphatase.

The second genetic modification can include increasing the expression of an endogenous trehalose-6-phosphate synthase (by providing an alternate promoter for example) and/or expressing an heterologous trehalose-6-phosphate synthase (by providing additional copies of the gene encoding the trehalose-6-phosphate synthase) in the recombinant yeast host cell. As used herein, the term “trehalose-6-phosphate synthase” refers to an enzyme capable of catalyzing the conversion of glucose-6-phosphate and UDP-D-glucose to α-α-trehalose-6-phosphate and UDP. In Saccharomyces cerevisiae, the trehalose-6-phosphate synthase gene can be referred to TPS1 (SGD:S000000330, Gene ID: 852423), BYP1, CIF1, FDP1, GGS1, GLC6 or TSS1. The recombinant yeast host cell of the present disclosure can include an heterologous nucleic acid molecule coding for TPS1, a variant thereof, a fragment thereof or for a polypeptide encoded by a TPS1 gene ortholog or paralog.

The second genetic modification can include increasing the expression of an endogenous trehalose-6-phosphate phosphatase (by providing an alternate promoter for example) and/or expressing an heterologous trehalose-6-phosphate phosphatase (by providing additional copies of the gene encoding the trehalose-6-phosphate phosphatase) in the recombinant yeast host cell. As also used herein, the term “trehalose-6-phosphate phosphatase” refers to an enzyme capable of catalyzing the conversion of α-α-trehalose-6-phosphate and H₂O into phosphate and trehalose. In Saccharomyces cerevisiae, the trehalose-6-phosphate phosphatase gene can be referred to TPS2 (SGD:S000002481, Gene ID: 851646), HOG2 or PFK3. The recombinant yeast host cell of the present disclosure can express an heterologous TPS2 (as well as a variant or a fragment thereof) from any origin including, but not limited to Saccharomyces cerevisiae (Gene ID: 851646), Arabidopsis thaliana (Gene ID: 838269), Schizosaccharomyces pombe (Gene ID: 2543109), Fusarium pseudograminearum (Gene ID: 20363081), Sugiyamaella lignohabitans (Gene ID: 30036691), Chlamydomonas reinhardtii (Gene ID: 5727896), Phaeodactylum tricornutum (Gene ID: 7194914), Candida albicans (Gene ID: 3636892), Kluyveromyces marxianus (Gene ID: 34714509), Scheffersomyces stipitis (Gene ID: 4840387), Spathaspora passalidarum (Gene ID: 18869689), Emiliania huxleyi (Gene ID: 17270873) or Pseudogymnoascus destructans (Gene ID: 36290309). The recombinant yeast host cell of the present disclosure can include a nucleic acid molecule coding for TPS2, a variant thereof, a fragment thereof or for a polypeptide encoded by a TPS2 gene ortholog or paralog. In a specific embodiments, the recombinant yeast host cell of the present disclosure includes a nucleic acid molecule encoding the amino acid sequence of SEQ ID NO: 46, a variant of the amino acid sequence of SEQ ID NO: 46 ora fragment of the amino acid sequence of SEQ ID NO: 46.

Alternatively or in combination, the second genetic modification can include increasing the expression of a polypeptide involved in regulating trehalose production (by providing an alternate promoter for example) or expression an heterologous polypeptide involved in regulating trehalose (by providing additional copies of the gene encoding the polypeptide). In Saccharomyces cerevisiae, polypeptides involved in regulating trehalose production include, but are not limited to TPS3 and TSL1. In some specific embodiment, the polypeptide involved in regulating trehalose production is TSL1. The recombinant yeast host cell of the present disclosure can express an heterologous TSL1 (as well as a variant or a fragment thereof) from any origin including, but not limited to Saccharomyces cerevisiae (SGD:S000004566, Gene ID 854872), Gallus gallus (Gene ID107050801), Kluyveromyces marxianus (Gene ID: 34714558), Saccharomyces eubayanus (Gene ID: 28933129), Schizosaccharomyces japonicus (Gene ID: 7049746), Pichia kudriavzevii (Gene ID: 31691677) or Hydra vulgaris (Gene ID 105848257). In a specific embodiments, the recombinant yeast host cell of the present disclosure includes a nucleic acid molecule encoding the amino acid sequence of SEQ ID NO: 45, a variant of the amino acid sequence of SEQ ID NO: 45 or a fragment of the amino acid sequence of SEQ ID NO: 45.

Additional Genetic Modifications

The recombinant yeast host cell of the present disclosure can also include one or more additional genetic modifications. These additional modifications can, for example, increase the fermentation abilities of the recombinant yeast host cell and, in some embodiments, increase ethanol yield and/or decrease glycerol yield of the recombinant yeast host cell during fermentation. In some embodiments, the recombinant yeast host cell can has a third genetic modification allowing or increasing the expression of an heterologous saccharolytic enzyme (with respect to a native yeast host cell lacking the third genetic modification); a fourth genetic modification allowing or increasing the production of formate/acetyl-CoA (when compared to a native yeast host cell lacking the fourth genetic modification); a fifth genetic modification allowing or increasing the utilization of acetyl-CoA (when compared to a native yeast host cell lacking the fifth genetic modification), a sixth genetic modification for reducing/limiting the production of glycerol (when compared to a native yeast host cell lacking the sixth genetic modification) and/or a seventh genetic modification for facilitating glycerol transport into the recombinant yeast host cell (when compared to a native yeast host cell lacking the seventh genetic modification). In an embodiment, the recombinant host cell has at least one of the third, fourth, fifth, sixth or seventh genetic modification. In another embodiment, the recombinant host cell has at least two of the third, fourth, fifth, sixth or seventh genetic modification. In an embodiment, the recombinant host cell has at least three of the third, fourth, fifth, sixth or seventh genetic modification. In an embodiment, the recombinant host cell has at least four of the third, fourth, fifth, sixth or seventh genetic modification. In an embodiment, the recombinant host cell has the third, fourth, fifth, sixth and seventh genetic modifications.

As indicated above, the recombinant yeast host cell can have a third genetic modification allowing the expression of an heterologous saccharolytic enzyme, such as a amylolytic enzyme. As used in the context of the present disclosure, a “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes. One embodiment of the saccharolytic enzyme is an amylolytic enzyme. As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase (GA) from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in WO2018/167670 and are incorporated herein by reference

In specific embodiments, the recombinant yeast host cell can bear one or more genetic modifications allowing for the production of an heterologous glucoamylase as the heterologous amylolytic enzyme. Many microbes produce an amylase to degrade extracellular starches. In addition to cleaving the last α(1-4) glycosidic linkages at the non-reducing end of amylose and amylopectin, yielding glucose, γ-amylase will cleave α(1-6) glycosidic linkages. The heterologous glucoamylase can be derived from any organism. In an embodiment, the heterologous polypeptide is derived from a γ-amylase, such as, for example, the glucoamylase of Saccharomycoces filbuligera (e.g., encoded by the glu 0111 gene). Examples of recombinant yeast host cells bearing such first genetic modifications are described in WO 2011/153516 as well as in WO 2017/037614 and herewith incorporated in its entirety. In an embodiment, the third genetic modification comprises introducing, in the recombinant yeast host cell, a nucleic acid molecule encoding the amino acid sequence of SEQ ID NO: 40, a variant of the amino acid sequence of SEQ ID NO: 40 or a fragment of the amino acid sequence of SEQ ID NO: 40. As such, the present disclosure provides a recombinant yeast host cell comprising a nucleic acid molecule encoding the amino acid sequence of SEQ ID NO: 40, a variant of the amino acid sequence of SEQ ID NO: 40 or a fragment of the amino acid sequence of SEQ ID NO: 40.

Alternatively or in combination, the recombinant yeast host cell can bear one or more fourth genetic modifications allowing or increasing the production of formate/acetyl-CoA. This can be achieved by promoting the conversion of pyruvate to acetyl-CoA and formate. In some specific embodiments, the recombinant yeast host cell can bear one or more genetic modifications allowing the expression of heterologous polypeptides having pyruvate formate lyase activity. As such, in some additional embodiments, the recombinant yeast host cell can include one or more further genetic modifications for increasing the production of an heterologous enzyme that function to anabolize (form) formate. As used in the context of the present disclosure, “an heterologous enzyme that function to anabolize formate” refers to polypeptides which may or may not be endogeneously found in the recombinant yeast host cell and that are purposefully introduced into the recombinant yeast host cells. In some embodiments, the heterologous enzyme that function to anabolize formate is an heterologous pyruvate formate lyase (PFL). Heterologous PFL of the present disclosure include, but are not limited to, the PFLA polypeptide, a polypeptide encoded by a pfla gene ortholog or paralog, the PFLB polyeptide or a polypeptide encoded by a pflb gene ortholog or paralog. In an embodiment, the fourth genetic modification comprises introducing, in the recombinant yeast host cell, a nucleic acid molecule encoding the amino acid sequence of SEQ ID NO: 42, a variant of the amino acid sequence of SEQ ID NO: 42 or a fragment of the amino acid sequence of SEQ ID NO: 42. As such, the present disclosure provides a recombinant yeast host cell comprising a nucleic acid molecule encoding the amino acid sequence of SEQ ID NO: 42, a variant of the amino acid sequence of SEQ ID NO: 42 or a fragment of the amino acid sequence of SEQ ID NO: 42. In an embodiment, the fourth genetic modification comprises introducing, in the recombinant yeast host cell, a nucleic acid molecule encoding the amino acid sequence of SEQ ID NO: 43, a variant of the amino acid sequence of SEQ ID NO: 43 or a fragment of the amino acid sequence of SEQ ID NO: 43. As such, the present disclosure provides a recombinant yeast host cell comprising a nucleic acid molecule encoding the amino acid sequence of SEQ ID NO: 43, a variant of the amino acid sequence of SEQ ID NO: 43 or a fragment of the amino acid sequence of SEQ ID NO: 43. In an embodiment, the fourth genetic modification comprises introducing, in the recombinant yeast host cell, one or more nucleic acid molecule encoding the amino acid sequence of SEQ ID NO: 42 and 43, a variant of the amino acid sequence of SEQ ID NO: 42 and 43 or a fragment of the amino acid sequence of SEQ ID NO: 42 and 43. As such, the present disclosure provides a recombinant yeast host cell comprising one or more nucleic acid molecule encoding the amino acid sequence of SEQ ID NO: 42 and 43, a variant of the amino acid sequence of SEQ ID NO: 42 and 43 or a fragment of the amino acid sequence of SEQ ID NO: 42 or 43. In an embodiment, recombinant yeast host cell bearing one of more fourth genetic modification can have native formate dehydrogenase (FDH) gene(s) (such as, for example, FDH1 and FDH2) and are capable of expressing the native FDH gene(s). In another embodiment, the recombinant yeast host cell bearing one or more fourth genetic modification can be further modified to have inactivated native FDH gene(s) (such as, for example, FDH1 and FDH2) and have a limited or no ability in expressing native FDH gene(s).

Alternatively or in combination, the recombinant yeast host cell can bear one or more fifth genetic modification allowing or increasing the utilization of acetyl-CoA. This can be achieved by promoting the conversion of acetyl-CoA to an alcohol like ethanol. In some specific embodiments, the recombinant yeast host cell can bear one or more genetic modifications allowing the expression of heterologous polypeptides having acetaldehyde dehydrogenase activity, alcohol dehydrogenase activity or both. In an heterologous acetaldehyde dehydrogenases (AADH), an heterologous alcohol dehydrogenases (ADH), and/or and heterologous bifunctional acetaldehyde/alcohol dehydrogenases (ADHE) such as those described in U.S. Pat. No. 8,956,851 and WO 2015/023989. More specifically, PFL and AADH enzymes for use in the recombinant yeast host cells can come from a bacterial or eukaryotic source. Heterologous AADHs of the present disclosure include, but are not limited to, the ADHE polypeptides or a polypeptide encoded by an adhe gene ortholog or paralog. In an embodiment, the fourth genetic modification comprises introducing, in the recombinant yeast host cell, a nucleic acid molecule encoding the amino acid sequence of SEQ ID NO: 44, a variant of the amino acid sequence of SEQ ID NO: 44 or a fragment of the amino acid sequence of SEQ ID NO: 44. As such, the present disclosure provides a recombinant yeast host cell comprising a nucleic acid molecule encoding the amino acid sequence of SEQ ID NO: 44, a variant of the amino acid sequence of SEQ ID NO: 44 or a fragment of the amino acid sequence of SEQ ID NO: 44.

The present disclosure comprises providing a recombinant yeast host cell having the fourth genetic modification but not the fifth genetic modification, the fifth genetic modification but not the fourth genetic modification as well as both the fourth and fifth genetic modification. In a specific embodiment, the recombinant comprises the fourth genetic modification (comprising one or more nucleic acid molecule for expressing an heterologous PFLA and PFLB) and the fifth genetic modification (comprising a nucleic acid molecule for expressing an heterologous ADHE).

Alternatively or in combination, the recombinant yeast host cell can also include one or more sixth genetic modifications limiting the production of glycerol. For example, the sixth genetic modification can be a genetic modification leading to the reduction in the production, and in an embodiment to the inhibition in the production, of one or more native enzymes that function to produce glycerol. As used in the context of the present disclosure, the expression “reducing the production of one or more native enzymes that function to produce glycerol” refers to a genetic modification which limits or impedes the expression of genes associated with one or more native polypeptides (in some embodiments enzymes) that function to produce glycerol, when compared to a corresponding yeast strain which does not bear such genetic modification. In some instances, the additional genetic modification reduces but still allows the production of one or more native polypeptides that function to produce glycerol. In other instances, the genetic modification inhibits the production of one or more native enzymes that function to produce glycerol. Polypeptides that function to produce glycerol refer to polypeptides which are endogenously found in the recombinant yeast host cell. Native enzymes that function to produce glycerol include, but are not limited to, the GPD1 and the GPD2 polypeptide (also referred to as GPD1 and GPD2 respectively) as well as the GPP1 and the GPP2 polypeptides (also referred to as GPP1 and GPP2 respectively). In an embodiment, the recombinant yeast host cell bears a genetic modification in at least one of the gpd1 gene (encoding the GPD1 polypeptide), the gpd2 gene (encoding the GPD2 polypeptide), the gppl gene (encoding the GPP1 polypeptide) or the gpp2 gene (encoding the GPP2 polypeptide). In another embodiment, the recombinant yeast host cell bears a genetic modification in at least two of the gpd1 gene (encoding the GPD1 polypeptide), the gpd2 gene (encoding the GPD2 polypeptide), the gppl gene (encoding the GPP1 polypeptide) or the gpp2 gene (encoding the GPP2 polypeptide). Examples of recombinant yeast host cells bearing such genetic modification(s) leading to the reduction in the production of one or more native enzymes that function to produce glycerol are described in WO 2012/138942. In some embodiments, the recombinant yeast host cell has a genetic modification (such as a genetic deletion or insertion) only in one enzyme that functions to produce glycerol, in the gpd2 gene, which would cause the host cell to have a knocked-out gpd2 gene. In some embodiments, the recombinant yeast host cell can have a genetic modification in the gpd1 gene and the gpd2 gene resulting is a recombinant yeast host cell being knock-out for the gpd1 gene and the gpd2 gene. In some specific embodiments, the recombinant yeast host cell can have be a knock-out for the gpd1 gene and have duplicate copies of the gpd2 gene (in some embodiments, under the control of the gpd1 promoter). In still another embodiment (in combination or alternative to the genetic modification described above).

In yet another embodiment, the recombinant yeast host cell does not bear a sixth genetic modification and includes its native genes coding for the GPP/GDP polypeptide(s).

Alternatively or in combination, the recombinant yeast host cell can also include one or more seventh genetic modifications facilitating the transport of glycerol in the recombinant yeast host cell. For example, the seventh genetic modification can be a genetic modification leading to the increase in activity of one or more native enzymes that function to transport glycerol. Native enzymes that function to transport glycerol synthesis include, but are not limited to, the FPS1 polypeptide as well as the STL1 polypeptide. The FPS1 polypeptide is a glycerol exporter and the STL1 polypeptide functions to import glycerol in the recombinant yeast host cell. By either reducing or inhibiting the expression of the FPS1 polypeptide and/or increasing the expression of the STL1 polypeptide, it is possible to control, to some extent, glycerol transport.

The STL1 polypeptide is natively expressed in yeasts and fungi, therefore the heterologous polypeptide functioning to import glycerol can be derived from yeasts and fungi. STL1 genes encoding the STL1 polypeptide include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID : 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID:19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus STL1 and Pichia sorbitophilia. In an embodiment, the STL1 polypeptide is encoded by Saccharomyces cerevisiae Gene ID: 852149. In an embodiment, the STL1 polypeptide has the amino acid sequence of SEQ ID NO: 39, is a variant of the amino acid sequence of SEQ ID NO: 39 or is a fragment of the amino acid sequence of SEQ ID NO: 39.

Process for Making a Fermented Product

The recombinant yeast host cells described herein can be used to improve fermentation yield, such as alcohol (e.g., ethanol) yield while maintaining yeast robustness during fermentation, even in the presence of a stressor, a bacterial contamination (that can be associated, in some embodiments, the an increase in lactic acid during fermentation), an increase in pH, a reduction in aeration, elevated temperatures or combinations. As shown herein, while the expression of the heterologous trehalase has the potential to increase ethanol production, it was shown to cause a reduction in robustness in the recombinant yeast host cell. This reduction in robustness was restored by introducing a second genetic modification for increase trehalose production.

The fermented product can be an alcohol, such as, for example, ethanol, isopropanol, n-propanol, 1-butanol, methanol, acetone and/or 1, 2 propanediol.

The present disclosure thus provides a recombinant yeast host cell which does increase trehalose production and also exhibits trehalase activity so as to maintain or increase the fermentation yield. In an embodiment, when a biomass (for example comprising corn) is fermented by the recombinant yeast host cell of the present disclosure, at the conclusion of a fermentation, the fermentation medium has less than 10 g/L, 9 g/L, 8 g/L, 7 g/L, 6 g/L, 5 g/L, 4 g/L, 3 g/L, 2 g/L or 1 g/L of glycerol. Alternatively or in combination, when a biomass (for example comprising corn) is fermented by the recombinant yeast host cell of the present disclosure, at the conclusion of a fermentation, the fermentation medium has less than 120 g/L, 110 g/L, 100 g/L, 90 g/L, 80 g/L, 70 g/L, 60 g/L, 50 g/L, 40 g/L, 30 g/L, 20 g/L or 10 g/L of glucose. Alternatively or in combination, when a biomass (for example comprising corn) is fermented by the recombinant yeast host cell of the present disclosure, at the conclusion of a permissive fermentation, the fermentation medium has at least 100 g/L, 105 g/L, 110 g/L, 115 g/L, 120 g/L, 125 g/L, 130 g/L, 135 g/L or 140 g/L of ethanol. Alternatively or in combination, when a biomass (for example comprising corn) is fermented by the recombinant yeast host cell of the present disclosure, at the conclusion of a stress fermentation, the fermentation medium has at least 50 g/L, 55 g/L, 60 g/L, 65 g/L, 70 g/L, 75 g/L, 80 g/L, 85 g/L or 90 g/L of ethanol.

The biomass that can be fermented with the recombinant yeast host cells described herein includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-polypeptide, extensin, and pro line-rich polypeptides).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASO), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

The process of the present disclosure contacting the recombinant host cells described herein with a biomass so as to allow the conversion of at least a part of the biomass into the fermentation product (e.g., an alcohol such as ethanol). In an embodiment, the biomass or substrate to be hydrolyzed is a lignocellulosic biomass and, in some embodiments, it comprises starch (in a gelatinized or raw form). The process can include, in some embodiments, heating the lignocellulosic biomass prior to fermentation to provide starch in a gelatinized form.

The fermentation process can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C. In some embodiments, the process can be conducted at temperatures above about 30° C., about 31° C., about 32° C., about 33° C., about 34° C., about 35° C., about 36° C., about 37° C., about 38° C., about 39° C., about 40° C., about 41° C., about 42° C., or about 50° C.

The fermentation process can be conducted, at least in part, in the presence of a stressor (such as high temperatures or the presence of a bacterial contamination).

In some embodiments, the process can be used to produce ethanol at a particular rate. For example, in some embodiments, ethanol is produced at a rate of at least about 0.1 g per hour per liter, at least about 0.25 g per hour per liter, at least about 0.5 g per hour per liter, at least about 0.75 g per hour per liter, at least about 1.0 g per hour per liter, at least about 2.0 g per hour per liter, at least about 5.0 g per hour per liter, at least about 10 g per hour per liter, at least about 15 g per hour per liter, at least about 20.0 g per hour per liter, at least about 25 g per hour per liter, at least about 30 g per hour per liter, at least about 50 g per hour per liter, at least about 100 g per hour per liter, at least about 200 g per hour per liter, or at least about 500 g per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The present invention will be more readily understood by referring to the following examples which are given to illustrate the invention rather than to limit its scope.

### EXAMPLE I

### Trehalase Screen

TABLE 1
Description of the trehalases used in the Examples

| | | | Nucleic | Amino | | |
|---|---|---|---|---|---|---|
| | | | acid - | acid - | | |
| | | | SEQ ID | SEQ ID | Strain # | Strain # |
| Reference | Source | Accession | NO: | NO: | (M2390) | (M15419) |
| MP244 | Aspergillus fumigatus | XP_748551 | 1 | 2 | M11245 | M16740, |
| | | | | | | M16742, |
| | | | | | | M16744 |
| MP1056 | Neosartorya udagawae | GAO81301 | 3 | 4 | M16289 | M16738 |
| MP1057 | Aspergillus flavus | XP_002380869 | 5 | 6 | M16291 | |
| MP1058 | Fusarium oxysporum | EMT72108 | 7 | 8 | | |
| MP1059 | Escovopsis weberi | KOS20950 | 9 | 10 | | |
| MP1060 | Microsporum gypseum | XP_003169590 | 11 | 12 | | |
| MP1061 | Aspergillus clavatus | XP_001273664 | 13 | 14 | | |
| MP1062 | Metarhizium anisopliae | KJK86671 | 15 | 16 | | |
| MP1063* | Ogataea parapolymorpha | XP_013934584 | 17 | 18 | | |
| MP1064* | Kluyveromyces marxianus | BAP73405 | 19 | 20 | M16293 | |
| MP1065* | Komagataella phaffii | CCA40810 | 21 | 22 | | |
| MP1066* | Ashbya gossypii | AAS54220 | 23 | 24 | M16295 | |
| MP1067 | Neurospora crassa | XP_965136 | 25 | 26 | M16283 | M16732, |
| | | | | | | M16746, |
| | | | | | | M16752, |
| | | | | | | M16753 |
| MP1068 | Thielavia terrestris | XP_003656356 | 27 | 28 | M16285 | M16734, |
| | | | | | | M16748, |
| | | | | | | M16750 |
| MP1069 | Aspergillus lentulus | GAQ05120 | 29 | 30 | M16287 | M16736 |
| MP1070* | Aspergillus ochraceoroseus | KKK15878 | 31 | 32 | | |
| MP1071 | Rhizoctonia solani | AGM46811 | 33 | 34 | | |
| MP1072 | Achlya hypogyna | AIG56056 | 35 | 36 | M16281 | M16731 |
| MP1073 | Schizopora paradoxa | KLO15949 | 37 | 38 | | |

*Trehalases lacking a signal sequence

Two copy expression cassettes (codon-optimized for S. cerevisiae) for each trehalases identified in Table 1 were engineered into the wildtype background strain M2390 under control of a constitutive promoter (TEF2p) and with their respective native signal peptide. Ten (10) clonal isolates were grown for 48 h in YPD medium and then the culture supernatants were incubated with 1% trehalose for 2 h prior to incubation with dinitrosalycilate (DNS). FIG. 2 displays the average trehalase activity for each enzyme relative to M2390 and MP244. Of the fifteen sequences assayed, eight had measurable activity higher than M2390 (MP1056, MP1057, MP1064, MP1066, MP1067, MP1068, MP1069 and MP1072).

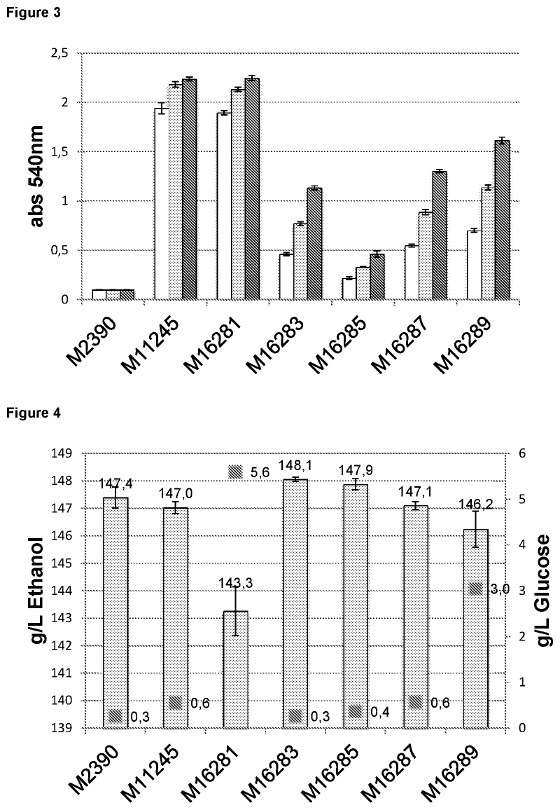

The trehalose assay was repeated using single colonies from the top five candidates. Single colonies of the top five candidates were grown in YPD for 48 h and then the culture supernatants were incubated with 1% trehalose for 30 min, 60 min, or 90 min prior to incubation with DNS. As shown in FIG. 3, under these conditions, MP244 (A. fumigatus trehalase expressed in strain M11245) and MP1072 (A. hypogynatrehalase expressed in strain M16281) had the highest secreted activity. MP1056 (N. udagawae trehalase in strain M16289) was the next highest, followed by MP1069 (A. lentulus trehalase in strain M16287), MP1067 (N. crassa trehalase in M16283) and MP1068 (T. terrestris trehalase in M16285).

The top five candidates expressing trehalases in strains M16281, M16283, M16285, M16287 and M16289 were subjected to either permissive or high temperature corn mash fermentation and compared to M2390 (wild-type) and M11245 (expressing the MP244 A. fumigatus trehalase). The permissive fermentation was run at 31.5% total solids (TS) containing 100% glucoamylase (GA at 0.6AGU/gTS) and 300 ppm urea at 33-31° C. (change at 20 h) in a CO₂ monitoring system. Conditions for high temperature fermentation were the same as permissive, but with the temperature held at 37° C. throughout. The 50 endpoint samples were submitted for HPLC analysis and measurement of trehalose using a Dionex column.

As can be seen in FIG. 4, strain M16283, expressing the N. crassa trehalase, gave an ˜0.5% ethanol increase relative to M2390. Strain M16285 also did quite well. At the end of the fermentation, the residual trehalose for strain M2390 was measured at 0.73 g/L. No detectable trehalose was measured for the engineered strains.

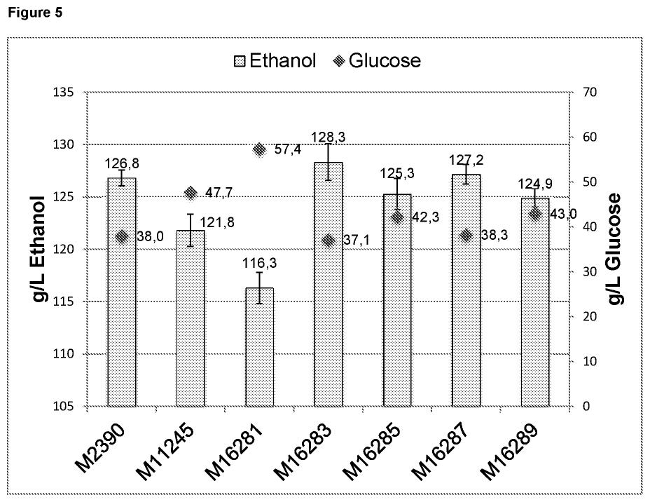

In terms of robustness at high temperatures, the N. crassa trehalase expressed in strain M16283 did not appear to lose robustness relative to strain M2390, which is an improvement from the current trehalase expressed in strain M11245 (FIG. 5). The other lower activity strains (M16285, M16287 and M16289) also perform similarly to M2390 (FIG. 5). Strains M11245 and M16281, the two highest activity strains, were the most temperature sensitive as can be seen by lower ethanol titers and higher residual glucose in the high temperature fermentation screen (FIG. 5). At the end of the fermentation, the residual trehalose for strain M2390 was measured at 0.6 g/L trehalose, wherease for the strain M16281, it was measured at 0.25 g/L. The remaining engineered strains did not show detectable trehalose amounts.

### EXAMPLE II

### Trehalase Combinations

The top five trehalase candidates identified in Example I (MP1072, MP1067, MP1068, MP1069 and MP1056) were also engineered in two copies under control of a constitutive promoter (TEF2p) and a terminator (ADH3t) either alone or in combination with overexpression of native TSL1 or TPS2 (trehalose regulatory or synthesis polypeptide) (TSL1 and TPS2 only with N. crassa or T. terrestris trehalase) as indicated in Tables 2A and B.

TABLE 2A
Description of the background strains used in this Example

| | Gene(s) | |
|---|---|---|
| | deleted | Gene(s) overexpressed |
| M2390 | None - wildtype strain | |
| M14926 | | STL1 (SEQ ID NO: 39), GA (SEQ ID NO: 40) |
| M4080 | | GA (SEQ ID NO: 40) |
| M15419 | fdh1Δ | FDH1 2 copies (SEQ ID NO: 41), |
| | fdh2Δ | PFLA (SEQ ID NO: 42), PFLB (SEQ ID NO: 43), |
| | gpd2Δ | ADHE (SEQ ID NO: 44), STL1 (SEQ ID NO: 39) |

TABLE 2B
Description of the strains used in this Example. GA = SEQ ID
NO: 40, TSL1 = SEQ ID NO: 45, STL1 = SEQ ID NO: 39, Formate =
PFLA (SEQ ID NO: 42), PFLB (SEQ ID NO: 43) and ADHE (SEQ ID
NO: 44), FDH1 = SEQ ID NO: 41, TPS2 = SEQ ID NO: 46.

| | Background strain | | | | | |
|---|---|---|---|---|---|---|
| | M14926 | | M4080 | | M2390 | |
| Trehalase | MP1068 | MP1067 | MP1068 | MP1067 | MP1068 | MP1067 |
| Other genes overexpressed | | | | | | |
| GA/TSL1 | | | | | | |
| GA/STL1/TSL1 | M17363 | M17512 | M17356 | M17502 | M17626 | |
| GA/STL1/Formate/TSL1 | M17513 | M17515 | M17504 | M17505 | | M17623 |
| GA/STL1/Formate/FDH1 | | | | | | |
| GA/STL1/Formate/TSL1/FDH1 | | | | | | M17621 |
| GA/STL1/Formate/FDH1/TPS2 | | | | | | |
| STL1/TSL1 | | | | | M17358 | M17562 |
| STL1/Formate/TSL1 | | | | | M17564 | M17566 |
| Formate/TSL1 | | | | | | |
| TSL1 | | | | | | |
| Trehalase only | | | | | M16285 | M16283 |

| | Background strain | | | | |
|---|---|---|---|---|---|
| | M2390 | M15419 | | | |
| Trehalase | MP1072 | MP1068 | MP1067 | MP1072 | MP244 |
| Other genes overexpressed | | | | | |
| GA/TSL1 | | | | | |
| GA/STL1/TSL1 | | | | | |
| GA/STL1/Formate/TSL1 | | | | | |
| GA/STL1/Formate/FDH1 | | | | M16731 | |
| GA/STL1/Formate/TSL1/FDH1 | | M16750 | M16752 | | M16742 |
| | | | M16753 | | |
| GA/STL1/Formate/FDH1/TPS2 | | M16748 | M16746 | | M16744 |
| STL1/TSL1 | | | | | |
| STL1/Formate/TSL1 | | | | | |
| Formate/TSL1 | | | | | |
| TSL1 | | | | | |
| Trehalase only | M16281 | | | | |

An initial fermentation screen was run to assess permissive and lactic stress performance of the strains compared to control strains. The fermentation was run at 32.5% TS, 33%, or 32.5% TS using mash under permissive, high temp stress, lactic acid (0.38% w/v of lactic acid added at 18 h, or bacterial stress conditions. Urea (300 ppm urea) was added in the permissive conditions only. Each yeast strains were dosed at 65% GA with 100% GA=0.6A GU/gTS. The permissive set was incubated at 33.3° C.-31° C. (temperature change was done at 18 h) for 50 h, the high temperatures set was incubated at 37° C. for 50 h and the bacterial stress set was incubated at 34° C. for 50 h. Lactobacillys plantarum (1.2^{E}9) was added up front for the bacterial stress condition.

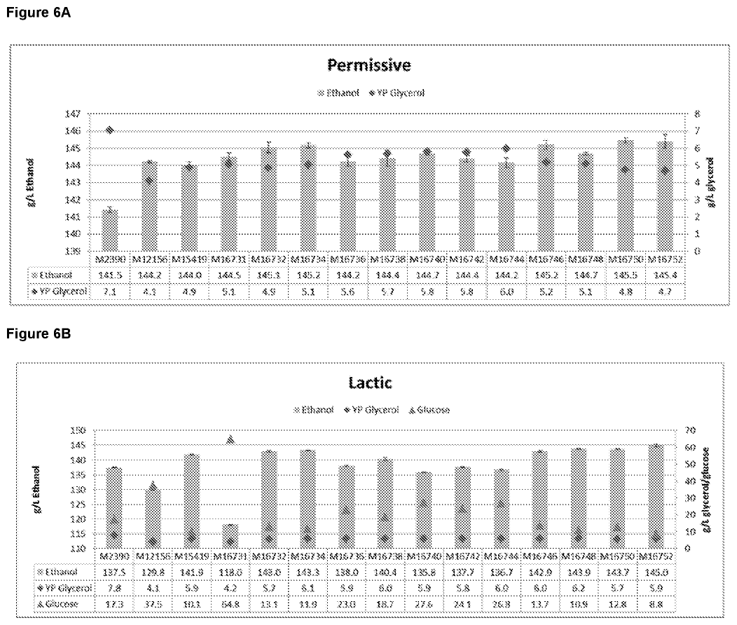

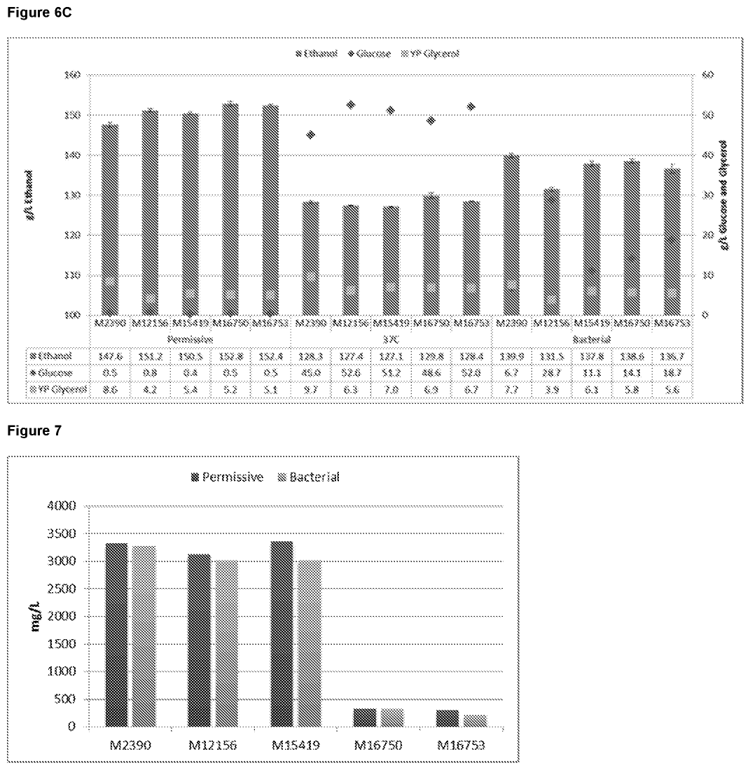

Strains expressing the N. crassa (M16752) or T. terrestris (M16750) trehalase in combination with TSL1 overexpression demonstrated a 1% yield increase relative to M15419 under permissive conditions and without loss in robustness under lactic stress or bacterial contamination (FIGS. 6A to 6C, Tables 3). The results presented therein show that strains capable of increasing trehalose production and expressing a trehalase are more robust (e.g., produce more ethanol, less glycerol and/or consume more glucose) than strains only expressing a trehalase.

TABLE 3A1
Additional results obtained after 50 h of permissive fermentation
conducted with 32.5% TS mash, 300 ppm urea, 65% GA for engineered
strains (100% = 0.6 AGU/gTS), 33-31° C., 150 rpm shaking.

| | GA | | | YP | Acetic | | | |
|---|---|---|---|---|---|---|---|---|
| Strains | Dose | Glucose | Lactic | Glycerol | Acid | Ethanol | Potential | Formate |
| M2390 | 100% | 0.6 | 0.3 | 8.5 | 0.6 | 148.7 | 148.9 | 0.000 |
| M12156 | 65% | 0.8 | 0.3 | 4.2 | 0.0 | 153.3 | 153.6 | 0.200 |
| M15419 | 65% | 0.4 | 0.4 | 5.3 | 0.1 | 151.5 | 151.7 | 0.000 |
| M17512 | 65% | 0.6 | 0.4 | 7.0 | 0.4 | 151.3 | 151.6 | 0.000 |
| M17513 | 65% | 2.6 | 0.3 | 3.8 | 0.0 | 153.7 | 154.9 | 0.155 |
| M17515 | 65% | 2.3 | 0.4 | 4.1 | 0.1 | 153.4 | 154.5 | 0.155 |
| M17502 | 65% | 0.8 | 0.4 | 6.8 | 0.5 | 150.6 | 151.0 | 0.000 |
| M17504 | 65% | 2.0 | 0.3 | 3.7 | 0.0 | 153.4 | 154.3 | 0.140 |
| M17505 | 65% | 3.9 | 0.3 | 3.6 | 0.0 | 151.3 | 153.1 | 0.155 |
| M17562 | 100% | 0.7 | 0.4 | 6.9 | 0.4 | 151.1 | 151.4 | 0.000 |
| M17564 | 100% | 3.1 | 0.4 | 4.7 | 0.1 | 152.1 | 153.6 | 0.135 |
| M17566 | 100% | 2.8 | 0.3 | 3.9 | 0.0 | 153.3 | 154.6 | 0.150 |

TABLE 3A2
Standard deviation of results of table 3A1.

| | | | YP | Acetic | | | |
|---|---|---|---|---|---|---|---|
| Strains | Glucose | Lactic | Glycerol | Acid | Ethanol | Potential | Formate |
| M2390 | 0.049 | 0.028 | 0.071 | 0.014 | 0.502 | 0.525 | 0.000 |
| M12156 | 0.014 | 0.007 | 0.035 | 0.000 | 0.297 | 0.303 | 0.000 |
| M15419 | 0.000 | 0.007 | 0.106 | 0.007 | 1.336 | 1.336 | 0.000 |
| M17512 | 0.007 | 0.000 | 0.042 | 0.000 | 0.460 | 0.463 | 0.000 |
| M17513 | 0.035 | 0.007 | 0.021 | 0.000 | 0.502 | 0.486 | 0.007 |
| M17515 | 0.071 | 0.000 | 0.021 | 0.021 | 0.255 | 0.222 | 0.007 |
| M17502 | 0.028 | 0.007 | 0.007 | 0.014 | 0.191 | 0.204 | 0.000 |
| M17504 | 0.021 | 0.007 | 0.028 | 0.000 | 0.014 | 0.024 | 0.000 |
| M17505 | 0.205 | 0.014 | 0.064 | 0.000 | 0.764 | 0.669 | 0.007 |
| M17562 | 0.014 | 0.021 | 0.071 | 0.127 | 0.325 | 0.319 | 0.000 |
| M17564 | 0.014 | 0.000 | 0.014 | 0.000 | 0.332 | 0.339 | 0.007 |
| M17566 | 0.057 | 0.000 | 0.014 | 0.000 | 0.085 | 0.111 | 0.000 |

TABLE 3B1
Additional results obtained after 50 h offermentation conducted under
permissive conditions (31.5% TS mash, 300 ppm urea, 65% GA for engineered
strains (100% = 0.6 AGU/gTS), 33-31° C., 150 rpm shaking;
EE = 2.5% TS mash, 400 ppm urea, 65% GA for engineered strains
(100% = 0.6 AGU/gTS), 33-31° C., 150 rpm shaking), lactic
conditions (31.5% TS mash, 65% GA for engineered strains (100% =
0.6 AGU/gTS), 34° C., 150 rpm shaking) or high temperature conditions
(33% TS mash, 0 ppm urea, 65% GA for engineered strains (100% =
0.6 AGU/gTS), 37° C., 150 rpm shaking).

| | | | | | YP | Acetic | | |
|---|---|---|---|---|---|---|---|---|
| Condition | GA | Strains | Glucose | Lactic | Glycerol | Acid | Ethanol | Formate |
| Permissive | 100% | M2390 | 0.44 | 0.32 | 8.69 | 0.61 | 140.21 | 0.00 |
| Lactic | 100% | M2390 | 2.64 | 3.76 | 8.57 | 0.66 | 137.96 | 0.00 |
| Permissive | 65% | M12156 | 0.69 | 0.22 | 4.46 | 0.00 | 143.13 | 0.15 |
| Lactic | 65% | M12156 | 41.02 | 4.05 | 4.35 | 0.14 | 121.11 | 0.11 |
| Permissive | 65% | M15419 | 0.26 | 0.30 | 5.43 | 0.12 | 142.45 | 0.00 |
| Lactic | 65% | M15419 | 0.35 | 3.99 | 5.99 | 0.11 | 140.17 | 0.00 |
| Permissive | 65% | M17356 | 0.32 | 0.33 | 7.00 | 0.32 | 142.19 | 0.00 |
| Lactic | 65% | M17356 | 8.60 | 4.06 | 6.41 | 0.30 | 137.60 | 0.00 |
| Permissive | 65% | M17363 | 0.46 | 0.31 | 7.32 | 0.37 | 140.28 | 0.00 |
| Lactic | 65% | M17363 | 8.01 | 3.91 | 6.55 | 0.31 | 138.37 | 0.00 |
| EE | 100% | M2390 | 0.2 | 0.5 | 9.4 | 0.5 | 144.9 | 0.0 |
| Permissive | | | | | | | | |
| Sterling | 100% | M2390 | 33.1 | 0.3 | 10.2 | 0.9 | 130.1 | 0.0 |
| Temp | | | | | | | | |
| EE | 50% | M12156 | 0.4 | 0.6 | 5.4 | 0.1 | 147.6 | 0.4 |
| Permissive | | | | | | | | |
| Sterling | 65% | M12156 | 48.7 | 0.3 | 6.1 | 0.3 | 125.0 | 0.0 |
| Temp | | | | | | | | |
| EE | 50% | M15419 | 0.2 | 0.6 | 6.3 | 0.2 | 146.7 | 0.1 |
| Permissive | | | | | | | | |
| Sterling | 65% | M15419 | 43.9 | 0.3 | 7.4 | 0.4 | 126.1 | 0.0 |
| Temp | | | | | | | | |
| EE | 50% | M17356 | 0.3 | 0.6 | 7.2 | 0.3 | 146.2 | 0.0 |
| Permissive | | | | | | | | |
| Sterling | 65% | M17356 | 38.6 | 0.3 | 7.7 | 0.5 | 130.4 | 0.0 |
| Temp | | | | | | | | |
| EE | 50% | M17363 | 0.3 | 0.4 | 7.5 | 0.4 | 147.7 | 0.0 |
| Permissive | | | | | | | | |
| Sterling | 65% | M17363 | 44.8 | 0.3 | 7.6 | 0.6 | 127.7 | 0.0 |
| Temp | | | | | | | | |

TABLE 3B2
Standard deviations of the results presented in table 3B1.

| | | | YP | Acetic | | |
|---|---|---|---|---|---|---|
| Strains | Glucose | Lactic | Glycerol | Acid | Ethanol | Formate |
| M2390 | 0.049 | 0.014 | 0.148 | 0.049 | 0.361 | 0.000 |
| M12156 | 0.085 | 0.276 | 0.064 | 0.007 | 0.304 | 0.000 |
| M15419 | 0.170 | 0.007 | 0.049 | 0.000 | 0.226 | 0.007 |
| M12156 | 2.680 | 0.170 | 0.028 | 0.000 | 1.633 | 0.000 |
| M15419 | 0.007 | 0.014 | 0.042 | 0.014 | 0.177 | 0.000 |
| M15419 | 0.021 | 0.127 | 0.028 | 0.014 | 1.153 | 0.000 |
| M17356 | 0.000 | 0.000 | 0.014 | 0.000 | 0.311 | 0.000 |
| M17356 | 0.163 | 0.007 | 0.049 | 0.007 | 0.311 | 0.000 |
| M17363 | 0.042 | 0.035 | 0.099 | 0.007 | 3.295 | 0.000 |
| M17363 | 1.259 | 0.134 | 0.007 | 0.000 | 0.764 | 0.000 |
| M2390 | 0.007 | 0.014 | 0.028 | 0.021 | 0.184 | 0.007 |
| M2390 | 2.157 | 0.007 | 0.057 | 0.014 | 0.990 | 0.000 |
| M2390 | 0.035 | 0.007 | 0.028 | 0.028 | 0.226 | 0.007 |
| M12156 | 0.502 | 0.007 | 0.007 | 0.007 | 0.629 | 0.000 |
| M12156 | 0.049 | 0.000 | 0.205 | 0.007 | 0.156 | 0.007 |
| M15419 | 0.580 | 0.007 | 0.000 | 0.000 | 0.035 | 0.000 |
| M15419 | 0.007 | 0.000 | 0.000 | 0.014 | 0.148 | 0.000 |
| M15419 | 0.396 | 0.021 | 0.148 | 0.007 | 0.361 | 0.000 |
| M17363 | 0.042 | 0.085 | 0.113 | 0.064 | 0.233 | 0.000 |
| M17363 | 0.856 | 0.007 | 0.127 | 0.014 | 0.205 | 0.000 |

TABLE 3C1
Additional results obtained after 50 h offermentation conducted under permissive
conditions (31.5% TS mash, 300 ppm urea, 100% GA 33-31° C., 150 rpm shaking),
or high temperature conditions (31.5% TS mash, 100% GA, 37° C., 150 rpm shaking).

| | | | | | YP | Acetic | | |
|---|---|---|---|---|---|---|---|---|
| Conditions | Strains | Glucose | Lactic | Glycerol | Glycerol | Acid | Ethanol | Formate |
| Permissive | M2390 | 0.3 | 0.4 | 10.5 | 8.0 | 0.4 | 147.4 | 0.00 |
| | M11245 | 0.6 | 0.3 | 11.1 | 8.7 | 0.5 | 147.0 | 0.00 |
| | M16281 | 5.6 | 0.3 | 12.6 | 10.1 | 0.4 | 143.3 | 0.00 |
| | M16283 | 0.3 | 0.4 | 10.4 | 8.0 | 0.4 | 148.1 | 0.00 |
| | M16285 | 0.4 | 0.3 | 10.6 | 8.2 | 0.4 | 147.9 | 0.00 |
| | M16287 | 0.6 | 0.3 | 11.1 | 8.6 | 0.4 | 147.1 | 0.00 |
| | M16289 | 3.0 | 0.3 | 11.3 | 8.8 | 0.5 | 146.2 | 0.00 |
| High Temp | M2390 | 38.0 | 0.4 | 11.2 | 8.8 | 0.8 | 126.8 | 0.00 |
| | M11245 | 47.7 | 0.3 | 12.6 | 10.1 | 1.0 | 121.8 | 0.00 |
| | M16281 | 57.4 | 0.3 | 12.6 | 10.2 | 0.9 | 116.3 | 0.02 |
| | M16283 | 37.1 | 0.4 | 11.2 | 8.8 | 0.8 | 128.3 | 0.00 |
| | M16285 | 42.3 | 0.4 | 11.3 | 8.8 | 0.8 | 125.3 | 0.00 |
| | M16287 | 38.3 | 0.3 | 11.7 | 9.3 | 0.9 | 127.2 | 0.00 |
| | M16289 | 43.0 | 0.3 | 11.7 | 9.2 | 0.9 | 124.9 | 0.00 |

TABLE 3C2
Standard deviations of the results presented in table 3C1.

| | | | YP | Acetic | | |
|---|---|---|---|---|---|---|
| Strains | Glucose | Lactic | Glycerol | Acid | Ethanol | Formate |
| M2390 | 0.021 | 0.028 | 0.014 | 0.035 | 0.382 | 0.000 |
| M11245 | 0.198 | 0.007 | 0.148 | 0.028 | 0.219 | 0.000 |
| M16281 | 1.160 | 0.007 | 0.382 | 0.042 | 0.884 | 0.000 |
| M16283 | 0.000 | 0.028 | 0.049 | 0.071 | 0.078 | 0.000 |
| M16285 | 0.042 | 0.007 | 0.092 | 0.021 | 0.205 | 0.000 |
| M16287 | 0.113 | 0.000 | 0.141 | 0.042 | 0.148 | 0.000 |
| M16289 | 0.969 | 0.007 | 0.092 | 0.014 | 0.658 | 0.000 |
| M2390 | 1.117 | 0.007 | 0.007 | 0.014 | 0.750 | 0.000 |
| M11245 | 3.026 | 0.000 | 0.014 | 0.007 | 1.541 | 0.000 |
| M16281 | 3.585 | 0.007 | 0.028 | 0.014 | 1.478 | 0.000 |
| M16283 | 3.330 | 0.000 | 0.057 | 0.007 | 1.747 | 0.000 |
| M16285 | 3.917 | 0.014 | 0.113 | 0.007 | 1.478 | 0.000 |
| M16287 | 2.220 | 0.000 | 0.007 | 0.014 | 0.940 | 0.000 |
| M16289 | 1.626 | 0.000 | 0.021 | 0.007 | 0.870 | 0.000 |

TABLE 3D1
Additional results obtained after 50 h offermentation conducted under permissive
conditions (32.5% TS mash, 300 ppm urea, GA as indicated in the table, 33-31°
C., 150 rpm shaking), lactic conditions (32.5% TS mash, 0 ppm urea, GA as indicated
in the table, 34° C., 0.38% w/v of lactic acid added at 18 h, 150 rpm
shaking) or high temperature conditions (33% TS mash, 0 ppm urea, 65% GA for
engineered strains (100% = 0.6 AGU/gTS), 37° C., 150 rpm shaking).

| | | | | | YP | Acetic | | |
|---|---|---|---|---|---|---|---|---|
| Condition | GA | Strains | Glucose | Lactic | Glycerol | Acid | Ethanol | Formate |
| Permissive | 100% | M2390 | 0.5 | 0.3 | 9.2 | 0.6 | 145.2 | 0.0 |
| Lactic | 100% | M2390 | 6.8 | 4.2 | 9.0 | 0.7 | 141.8 | 0.0 |
| Permissive | 65% | M12156 | 0.4 | 0.3 | 4.9 | 0.1 | 148.9 | 0.3 |
| Lactic | 65% | M12156 | 43.7 | 4.1 | 4.4 | 0.1 | 125.7 | 0.2 |
| Permissive | 65% | M15419 | 0.2 | 0.3 | 5.8 | 0.2 | 148.3 | 0.0 |
| Lactic | 65% | M15419 | 5.3 | 4.2 | 6.4 | 0.2 | 143.0 | 0.0 |
| Permissive | 65% | M17621 | 0.2 | 0.3 | 6.8 | 0.5 | 148.3 | 0.0 |
| Lactic | 65% | M17621 | 22.9 | 4.2 | 6.8 | 0.3 | 135.9 | 0.0 |
| Permissive | 65% | M17623 | 0.8 | 0.3 | 5.5 | 0.2 | 150.2 | 0.2 |
| Lactic | 65% | M17623 | 38.4 | 4.1 | 5.1 | 0.1 | 130.2 | 0.1 |
| Permissive | 65% | M17626 | 0.4 | 0.3 | 7.4 | 0.4 | 149.1 | 0.0 |
| Lactic | 65% | M17626 | 24.4 | 4.2 | 6.5 | 0.4 | 136.7 | 0.0 |

TABLE 3D2
Standard deviations of the results presented in table 3D1.

| | | | YP | Acetic | | |
|---|---|---|---|---|---|---|
| Strains | Glucose | Lactic | Glycerol | Acid | Ethanol | Formate |
| M2390 | 0.021 | 0.007 | 0.078 | 0.021 | 0.750 | 0.000 |
| M2390 | 0.820 | 0.028 | 0.092 | 0.000 | 0.290 | 0.000 |
| M12156 | 0.000 | 0.000 | 0.042 | 0.014 | 0.622 | 0.007 |
| M12156 | 0.742 | 0.049 | 0.057 | 0.042 | 0.042 | 0.007 |
| M15419 | 0.014 | 0.035 | 0.064 | 0.042 | 0.212 | 0.014 |
| M15419 | 2.001 | 0.198 | 0.113 | 0.042 | 1.803 | 0.000 |
| M17621 | 0.007 | 0.000 | 0.071 | 0.049 | 0.679 | 0.000 |
| M17621 | 1.202 | 0.099 | 0.042 | 0.021 | 0.417 | 0.000 |
| M17623 | 0.014 | 0.007 | 0.007 | 0.007 | 0.205 | 0.000 |
| M17623 | 0.948 | 0.042 | 0.078 | 0.014 | 0.799 | 0.000 |
| M17626 | 0.021 | 0.035 | 0.028 | 0.021 | 0.092 | 0.000 |
| M17626 | 0.290 | 0.071 | 0.035 | 0.057 | 0.106 | 0.000 |

A secondary fermentation was run to compare the sibling colony of M16752, M16753, which performed slightly better and was selected for further studies (data not shown).

Additional fermentations were performed to evaluate M16750 and M16753 under higher solids, high temperature or bacterial stress conditions. Results are summarized in FIG. 6. Both strains appear to give ˜1% yield increase relative to both M12156 and M15419. In addition, these strains maintain temperature and bacterial stress tolerance compared to M15419.

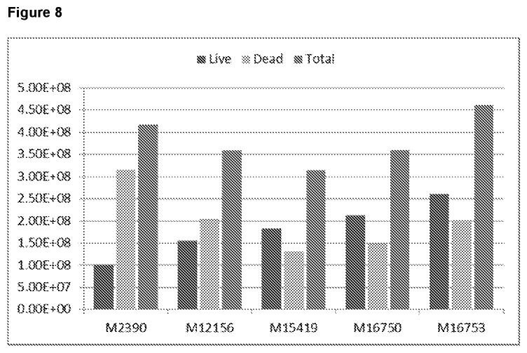

Two strains were further evaluated to quantify trehalose at the end of fermentation. Fermentation supernatants (of permissive and bacterial fermentations) were run on the Dionex and demonstrated a reduction in trehalose relative to the control strains (FIG. 7). Furthermore, end of permissive fermentation samples were analyzed for live and dead cells via methylene blue staining and cell counting on hemocytometer. As shown on FIG. 8, strains M16750 and M16753 were shown to have similar cell counts as M15419.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

An M Z, Tang Y Q, Mitsumasu K, Liu Z S, Shigeru M, Kenji K. Enhanced thermotolerance for ethanol fermentation of Saccharomyces cerevisiae strain by overexpression of the gene coding for trehalose-6-phosphate synthase. Biotechnol Lett. 2011 July; 33(7):1367-74.

Bell W, Sun W, Hohmann S, Wera S, Reinders A, De Virgilio C, Wiemken A, Thevelein J M. Composition and functional analysis of the Saccharomyces cerevisiae trehalose synthase complex. J Biol Chem. 1998 Dec. 11; 273(50):33311-9.

Cao T S, Chi Z, Liu G L, Chi Z M. Expression of TPS1 gene from Saccharomycopsis fibuligera A11 in Saccharomyces sp. W0 enhances trehalose accumulation, ethanol tolerance, and ethanol production. Mol Biotechnol. 2014 January; 56(1):72-8.

Ge X Y, Xu Y, Chen X. Improve carbon metabolic flux in Saccharomyces cerevisiae at high temperature by overexpressed TSL1 gene. J Ind Microbiol Biotechnol. 2013 April; 40(3-4):345-52.

Guo Z P, Zhang L, Ding Z Y, Shi G Y. Minimization of glycerol synthesis in industrial ethanol yeast without influencing its fermentation performance. Metab Eng. 2011 January; 13(1):49-59.

Thevelein J M, Hohmann S. Trehalose synthase: guard to the gate of glycolysis in yeast? Trends Biochem Sci. 1995 Jan; 20(1):3-10.

1. A recombinant yeast host cell having:
a first genetic modification for expressing an heterologous trehalase; and
a second genetic modification for increasing trehalose production.

2. The recombinant yeast host cell of claim 1, wherein the heterologous trehalase is a cell-associated trehalase.

3. The recombinant yeast host cell of claim 1, wherein the heterolgous trehalase is a secreted trehalase.

4. The recombinant yeast host cell of claim 1, wherein the heterologous trehalase:
(a) has the amino acid sequence of SEQ ID NO.: 26, 2, 4, 6, 8, 10, 12, 14, 16, 18, 20, 22, 24, 28, 30, 32, 34, 36 or 38;
(b) is a variant of the amino acid sequence of (a) exhibiting trehalase activity; or
(c) is a fragment of the amino acid sequence of (a) or (b) exhibiting trehalase activity.

5. The recombinant yeast host cell of claim 1, wherein the heterologous trehalase is from Neurospora sp., Achlya sp., Ashbya sp., Aspergillus sp., Escovopsis sp., Fusarium sp., Kluyveromyces sp., Komagataella sp., Metarhizium sp., Microsporum sp., Neosartorya sp., Ogataea sp., Rhizoctonia sp., Schizopora sp., or Thielavia sp.

6. The recombinant yeast host cell of claim 5, wherein the heterologous trehalase is from Neurospora crassa, Achlya hypogyna, Ashbya gossypii, Aspergillus clavatus, Aspergillus flavus, Aspergillus fumigatus, Aspergillus lentulus, Aspergillus ochraceoroseus, Escovopsis weberi, Fusarium oxysporum, Kluyveromyces marxianus, Komagataella phaffii, Metarhizium anisopliae, Microsporum gypseum, Neosartorya udagawae, Ogataea parapolymorpha, Rhizoctonia solani, Schizopora paradoxa, or Thielavia terrestris.

7.-57. (canceled)

58. The recombinant yeast host cell of claim 1, wherein the second genetic modification allows (i) expression of a second heterologous enzyme involved in producing trehalose and/or a second heterologous regulatory polypeptide involved in regulating trehalose production and/or (ii) overexpression of a second native enzyme involved in producing trehalose and/or a second native regulatory polypeptide involved in regulating trehalose production.

59. (canceled)

60. The recombinant yeast host cell of claim 58, wherein the second genetic modification allows the expression of at least one of TPS1, TPS2, TPS3 or TSL1.

61.-64. (canceled)

65. The recombinant yeast host cell of claim 1 which exhibit increased robustness when a stressor is present, compared to a corresponding recombinant yeast host cell having the first genetic modification and lacking the second genetic modification.

66. The recombinant yeast host cell of claim 1 further comprising at least one of:
a third genetic modification allowing or increasing expression of at least one heterologous saccharolytic enzyme;
a fourth genetic modification allowing or increasing production of formate;
a fifth genetic modification allowing or increasing utilization of acetyl-CoA;
a sixth genetic modification limiting production of glycerol; and/or a seventh genetic modification facilitating transport of glycerol in the recombinant yeast host cell.

67. The recombinant yeast host cell of claim 1 which belongs to a species from genus Saccharomyces sp.

68. The recombinant yeast host cell of claim 67 wherein the species is Saccharomyces cerevisiae.

69. A process for converting a biomass into a fermentation product, the process comprising contacting the biomass with the recombinant yeast host cell defined in claim 1 under conditions to allow conversion of at least a part of the biomass into the fermentation product.

70. The process of claim 69, wherein the biomass comprises corn.

71. The process of claim 70, wherein the corn is provided as a mash.

72. The process of claim 69, wherein the fermentation product is ethanol.

73. The process of claim 69 being conducted, at least in part, with a stressor present.


SEQUENCE LISTING (46 sequences)

SEQ ID NO: 1 - DNA, 3216 bp, Artificial Sequence
  Codon optimized sequence for yeast expression
atgttgtctcaaaacttggctacttgggtttctttgttggcttgtttgccagctgctatcggtttgccaaacaacaacgacagagttgctagatctttgaagagacacggtggtcacggtcacaagcaagttgacactaactcttctcacgtttacaagactagattcccaggtgttacttgggacgacgaccactggttgttgtctactactactttggaccaaggtcactaccaatctagaggttctatcgctaacggttacttgggtatcaacgttgcttctgttggtccattcttcgaattggacgttccagtttctggtgacgttatcaacggttggccattgtactctagaagacaaactttcgctactatcgctggtttcttcgactaccaaccaactactaacggttctaacttcccatggttgaaccaatacggtggtgaatctgttatctctggtatcccacactggtctggtttgatcttggacttgggtgacggtaactacttggacgctactgttgacaacaagactatcactgacttcagatctacttacgacttcaagtctggtgttttgtcttggtcttacacttggactccaaagtgtaacaagggttctttcaacatcacttacagattgttcgctcacaagttgcacgttaaccaagctgttgttgacatggaaatcactccatctcaaggttctgaagctactgttgttaacgttatcgacggtttctctgctgttagaactgacttcgttgaatctggtcaagacaacggtgctttgttctctgctgttagaccatggggtatctctaacgttactgcttacgtttacactaacttgactgcttctgctggtgttgacttgacttctagagctttggttaacgacaagccatacgttcactctaacgaatcttctatcgctcaagctgttgacgttaagttcagagctaacgaaactgttagaatcactaagttcgttggtgctgcttcttctgacgctttcccaaacccacaacaaactgctaagcaagctgtttctgctgctatgggtgctggttacatgggttctttgcaatctcacgttgaagaatgggcttctatcttgttggacggttctgttgactctttcgttgacccagctactggtaagttgccagacgacgaccacatcttgaactctcaaatcatcgctgttgctaacacttactacttgttgcaaaacactgttggtaagaacgctatcaaggctgtttctggtgctccagttaacgttgactctatctctgttggtggtttgacttctgactcttacgctggtttggttttctgggacgctgacgtttggatgcaaccaggtttggttgcttctcacccagaagctgctcaaagagttactaactacagaactaagttgtacccacaagctttggaaaacatcaacactgctttcacttcttctaagaacagaactactttctctccatctgctgctatctacccatggacttctggtagattcggtaactgtactggtactggtccatgttgggactaccaataccacttgaacggtgacatcggtttgtctttgatgtaccaatggatcgcttctggtgacactaagactttcagagaacaacacttcccaatctacgactctgttgctactatgtactctaacatcgttcaaagaaacggttcttcttggactttgactaacatgactgacccagacgaatacgctaaccacatcgacgctggtggtttcactatgccattgatctctgaaactttgtcttacgctaactctttcagaaagcaattcggtttggaacaaaacgaaacttggactgaaatctctgaaaacgttttgttgatcagagaagacggtgttactttggaatacactactatgaacggtactgctgttgttaagcaagctgacatcgttttggttacttacccattggtttacgacaacaactacactgctcaacacgctttgaacgacttggactactacgctaaccaacaatctccagacggtccagctatgacttgggctatcttcgctatcactgctaacgacgtttctccatctggttgttctgcttacacttaccaccaagactcttacgacccatacatgagagctccattctaccaattgtctgaacaaatgatcgacgacgctggtatcaacggtggtactcacccagcttacccattcttgactggtcacggtggtgctaaccaagttgttttgatgggttacttgggtttgagattgttgccagacgacgctatccacatcgacccaaacttgccaccacaagtttctaacttgaagtacagaactttctactggagaggttggccaatctcttcttcttctaacagaactcacactactatctctagagctgctaacttggctccattggacactgctgactctagattcgctaacgcttctatcccagttttggttggtgacccatctaactctactgcttacagattgccagttactgctccattggttgttccaaacagacaaatcggtttcaacaacactatcccaggtaacatggttcaatgtagaccagtttactctccaaacgactacgctccaggtcaattcccaatcgctgctgttgacggtgctacttctactaagtggagaccatctactgctaacatgtcttctttgactgttgctttggctgacgttgaaatcaactctaaggtttctggtttccacttcaactggtggcaagctccaccagttaacgctactgttatcttccacgacgaaatgttggaagacccagttgctgctatgtcttcttctcacggtaactctagatacagagttgttactactttgactaacatcgaacaatctcaaccatacgacgctcaatctactgacaacaacgaagttgttttgaacactggtaacactactgacgtttctttgtctcaaactgttcacacttctagatacgctactttgttgatctctggtaaccaagctggtggtgaagaaggtgctactgttgctgaatgggctatcttgggtgaatctaagggttcttcttctggtcacggtaacaacaagagaagattggacgttagagctgctgctgctttgtctgctttgaacgacagaagatacagacaattcaacgct

SEQ ID NO: 2 - protein, 1072 aa, Aspergillus fumigatus
MLSQNLATWVSLLACLPAAIGLPNNNDRVARSLKRHGGHGHKQVDTNSSHVYKTRFPGVTWDDDHWLLSTTTLDQGHYQSRGSIANGYLGINVASVGPFFELDVPVSGDVINGWPLYSRRQTFATIAGFFDYQPTTNGSNFPWLNQYGGESVISGIPHWSGLILDLGDGNYLDATVDNKTITDFRSTYDFKSGVLSWSYTWTPKCNKGSFNITYRLFAHKLHVNQAVVDMEITPSQGSEATVVNVIDGFSAVRTDFVESGQDNGALFSAVRPWGISNVTAYVYTNLTASAGVDLTSRALVNDKPYVHSNESSIAQAVDVKFRANETVRITKFVGAASSDAFPNPQQTAKQAVSAAMGAGYMGSLQSHVEEWASILLDGSVDSFVDPATGKLPDDDHILNSQIIAVANTYYLLQNTVGKNAIKAVSGAPVNVDSISVGGLTSDSYAGLVFWDADVWMQPGLVASHPEAAQRVTNYRTKLYPQALENINTAFTSSKNRTTFSPSAAIYPWTSGRFGNCTGTGPCWDYQYHLNGDIGLSLMYQWIASGDTKTFREQHFPIYDSVATMYSNIVQRNGSSWTLTNMTDPDEYANHIDAGGFTMPLISETLSYANSFRKQFGLEQNETWTEISENVLLIREDGVTLEYTTMNGTAVVKQADIVLVTYPLVYDNNYTAQHALNDLDYYANQQSPDGPAMTWAIFAITANDVSPSGCSAYTYHQDSYDPYMRAPFYQLSEQMIDDAGINGGTHPAYPFLTGHGGANQVVLMGYLGLRLLPDDAIHIDPNLPPQVSNLKYRTFYWRGWPISSSSNRTHTTISRAANLAPLDTADSRFANASIPVLVGDPSNSTAYRLPVTAPLVVPNRQIGFNNTIPGNMVQCRPVYSPNDYAPGQFPIAAVDGATSTKWRPSTANMSSLTVALADVEINSKVSGFHFNWWQAPPVNATVIFHDEMLEDPVAAMSSSHGNSRYRVVTTLTNIEQSQPYDAQSTDNNEVVLNTGNTTDVSLSQTVHTSRYATLLISGNQAGGEEGATVAEWAILGESKGSSSGHGNNKRRLDVRAAAALSALNDRRYRQFNA

SEQ ID NO: 3 - DNA, 3219 bp, Neosartorya udagawae
atgttgtccaagaatttggctacctgggtttctttgttggcttgtttgccagctactattggtttgccaaacaacaatggtagaatcgccagatctttgaaaagacatggtggtcatggtcaaaagcaagttgataccaattcctctcatgtttacgataccagatttccaggtgttacttgggatgatgatcattggttgttgtctactactaccttggatcaaggtcactatcaatccagaggttctattgctaatggttatttgggtattaacgttgcctctgttggtccatttttcgaattggatgttccagttggtggtgatgttattaacggttggccattatactctagaagacaaacttttgctaccattgccggtttcttcgattatcaaccagctaccaatggttctaatttcccatggttgaatcaatacggtggtgaatctgttatttccggtattccacattggtccggtttgattttggatttgggtaatggtacttacttggatgctaccgttgataacaagaccattaccgatttcagatccacctacgattttaagtctggtgttttgtcttggtcttacacttggactccaacttgtaacaagggttctttcaacatcacctacagattattcgcccataagttgcatgttaatcaagccgttgttgacatggaaattactccatctcaaggttctcaagccactgttgttaatgttatcgatggttactctgccgttagaaccgattttgttgaatctggtcaagataacggtgctatcttttctgctgttagaccatggggtatttctaacgttactgcttacgtctataccaacttgactgcttctgctggtgttgatttgtcatcaagagctttggttaacgataagccatacgttcactctaacgaatcctctattgctcaagctgttaacgtcaagttttctgccaacgaaaccattagaatcaccaagtttgttggtgctgcttcttctgatgcttttccaaatcctcaacaaactgctaagcaagctgtttctgctgctatgggtgctggttatatgggttctttacaatctcatgttgctgaatgggcctccattttgttggatggttctgttgattctttcgttgatccagctactggtaaattgccagatgatgaacatatcttgaactcccaaattattgctgttgccaacacctactacttgttgcaaaacactgttggtaagaacgctattaaggctgtttcaggtgctccagttaatgtcaactctatttctgttggtggtttgacctctgattcttatgctggtttggttttttgggatgctgatgtttggatgcaaccaggtttggttgcttctcatccagaagctgctcaaagagttactaattacagaactaagttgtacccacaagccttggaaaacatcaatactgctttcacctcctctaagaatcaaacctctttttctccatctgctgcaatctatccatggacatctggtagatttggtaactgtactggtactggtccatgttgggattatcaatatcatttggatggtgacatcggtttgtccttgatgtatcaatggattgcttctggtgatacccaaacctttagagaacaacatttcccaatctacgattccattgccactatgtactctaacatcgttcaaagaaatggttcctcttggactttgactaacatgactgatccagatgaatacgccaatcatgttgatggtggtggttttactatgccattgatttctgaaactttgggttacgccaactccttcagaaaacaatttggtttggaacaaaacgaaacctgggccgaaatttccgaaaatgttttggttatcagagaaaacggtgtcaccatggaatacactactatgaatggtactaccgttgtcaagcaagcagatgttgttttagttacctacccattggtttacgacaacaactatactgcccaagactccttgaatgatttggattactacgccaatagacaatctccagatggtcctgctatgacttgggctatttttgctattactgctaacgatgtttccccatctggttgttctgcttttacttaccatcaaaactcctacgacccatatatgagagcaccattttaccaattgtccgaacaaatgttggatgaagcctctatcaatggtggtactcatccagcttatccatttttgacaggtcatggtggtgctaatcaagttgttttgttcggttacttaggtttgagattattgccagacgacgccattcatattgatccaaatttgccaccacaagtctctaacgttgcttacagaacattttattggagaggttggccaatttccgcttcttctaatagaactcataccaccatatctagagctgctaatgttgaacctttggatactgctgattctagatttgctaacgccaccatttcagttttggttggtgatccatctaattccaccgcttatcaattaccagctacaggtccattagttgtcccaaatagacaaatcggtttcaacaacactatcccaggtaacatggttcaatgtagaccagtttattccccacatgattatgttccaggtcaatttccaattgctgctgttgacggtgctacttctacaaaatggcaaccatctactgccaacatgtcatctttgactgttgctttggcagacatcgaaatcaactctaaggtttctggtttccatttcaattggtggcaagctccacctgttaatgctactgttattttccacgatgaagttttggaagatccagttgctgcaatgtcatctgctcatggtaattctcaatacaagatcgttaccaccttgaccaacatcgaacaatctcaaccatacaatgctcaaggtactgattacaatgttgttgctatgtctaccggtaacactactgaagtcaatttgtctcaaaccgttcacacttctagatacgctaccttgttgatctctggtaatcaaggtggtggtgaaaaaggtgctacagtagcagaatgggcaattttgggtgaatcaaaaggttcttcttccggtcatggtaacaacaagagaagattggatgtaagagctgctgctgctttgtctggtggtttaaatgatagaagatggcaacaattcaatgct

SEQ ID NO: 4 - protein, 1073 aa, Neosartorya udagawae
MLSKNLATWVSLLACLPATIGLPNNNGRIARSLKRHGGHGQKQVDTNSSHVYDTRFPGVTWDDDHWLLSTTTLDQGHYQSRGSIANGYLGINVASVGPFFELDVPVGGDVINGWPLYSRRQTFATIAGFFDYQPATNGSNFPWLNQYGGESVISGIPHWSGLILDLGNGTYLDATVDNKTITDFRSTYDFKSGVLSWSYTWTPTCNKGSFNITYRLFAHKLHVNQAVVDMEITPSQGSQATVVNVIDGYSAVRTDFVESGQDNGAIFSAVRPWGISNVTAYVYTNLTASAGVDLSSRALVNDKPYVHSNESSIAQAVNVKFSANETIRITKFVGAASSDAFPNPQQTAKQAVSAAMGAGYMGSLQSHVAEWASILLDGSVDSFVDPATGKLPDDEHILNSQIIAVANTYYLLQNTVGKNAIKAVSGAPVNVNSISVGGLTSDSYAGLVFWDADVWMQPGLVASHPEAAQRVTNYRTKLYPQALENINTAFTSSKNQTSFSPSAAIYPWTSGRFGNCTGTGPCWDYQYHLDGDIGLSLMYQWIASGDTQTFREQHFPIYDSIATMYSNIVQRNGSSWTLTNMTDPDEYANHVDGGGFTMPLISETLGYANSFRKQFGLEQNETWAEISENVLVIRENGVTMEYTTMNGTTVVKQADVVLVTYPLVYDNNYTAQDSLNDLDYYANRQSPDGPAMTWAIFAITANDVSPSGCSAFTYHQNSYDPYMRAPFYQLSEQMLDEASINGGTHPAYPFLTGHGGANQVVLFGYLGLRLLPDDAIHIDPNLPPQVSNVAYRTFYWRGWPISASSNRTHTTISRAANVEPLDTADSRFANATISVLVGDPSNSTAYQLPATGPLVVPNRQIGFNNTIPGNMVQCRPVYSPHDYVPGQFPIAAVDGATSTKWQPSTANMSSLTVALADIEINSKVSGFHFNWWQAPPVNATVIFHDEVLEDPVAAMSSAHGNSQYKIVTTLTNIEQSQPYNAQGTDYNVVAMSTGNTTEVNLSQTVHTSRYATLLISGNQGGGEKGATVAEWAILGESKGSSSGHGNNKRRLDVRAAAALSGGLNDRRWQQFNA

SEQ ID NO: 5 - protein, 3222 aa, Aspergillus flavus
ATGAAGTTGAGAAATTTGGCTCCATGGGCTTTGTTGTTGACTGCTGTTCATGGTTTGCCATCTCAAGGTACTCAAAACAAACATAATCCAAGAGTCGCCAAGATTTTGAAGAGACATGAAGGTTCTTCTCAAAAGGCCAAGGATTCTAACAACGTCTACGAAACTAAGTTCGATGGTGTTACTTGGGATGAAGAAAACTGGTTGTTGAAAACTACCACCTTGGATCAAGGTCACTATCAATCTAGAGGTTCTGTTGCTAATGGTTATTTGGGTATTAACGTTGCTTCTGTCGGTCCATTTTTCGAATTGGACGAAGAAGTTGATGGTGATGTTATTAACGGTTGGCCATTATACTCCAGAAGACAATCTTTTGCTACCATTGCTGGTTTCTTCGATTCTCAACCTACTACCAATGGTACTAATTTCCCATGGTTGTCTCAATACGGTTGGGATACTGCTATTTCTGGTGTTCCACATTGGTCTGGTTTGATTTTGGATTTGGGTGATGATGTCTACTTGGATTCTACCGTTGATGATTCTACCATCACCGATTTCCAATCTACCTACGATTTTAAGGCTGGTGTTTTGTCTTGGTCTTATACTTGGTCACCAGCTGATAAGGGTTCTTTCGAAATTACCTACAGATTATTCGCCAACAAGTTGAACATTACCCAAGCCGTTGTTGACATGGAAATTATCCCATCTGTTGATGCTAATGCTACCGTTGCTAACGTTATTGATGGTTACTCTGCTGTTAGAACCGATTTCGTTGAATCTGGTCAAGATGATGGTGCTTTGTTTTCAGCTGTTAGACCATGGGGTATTTCTAACGTTACTGCTTACATCTACACTAACTTGACTGGTTCCGCTAACGTTGATTTGTCATCTAGAGCTTTGGTTACTGGTAAGCCATACGTCAATACCAACGAATCTTCAGTTGCTCAAACTGTCAACGTTAAGTTCACTGCTAAAGAACCAGTTAGAATCACCAAGTTTGTTGGTGGTGCTTCTACTGATGCTTTTGCTGATCCTAAACAAACCGCTAAAGAAGCTGCTTCTGCTGCTTTGGCTGCTGGTTACAAAAATTCTTTGGAATCCCATGCTTCTGAATGGGCTAACATTATGCACGAAAACTCTGTTGATAGATTCACTGATCCAACCACTGGTAAATTGCCTGAAGATCAACATGTTATCGATTCCGCTGTTATTGCTGTTACCAACATCTACTACTTGTTGCAAAACACCGTTTCCCAAAATGCTATTGCTGCTGTTTCAAACGCTACTGTTAACGAAACTTCTTTCTCCGTTGGTGGTTTGACTTCTGATTCTTATGGTGGTCAAGTTTTCTGGGATGCTGATGTTTGGATGCAACCAGGTTTGGTTGCTTCTCATCCTGAAGCTGCTCAAGGTGTTACTAATTACAGAGTTGCTAAGTACCAACAAGCCAAAGAAAACGTTAAGACTGCTTTCACCTCCTCTAAGAATCAAACTAGATTCGATCCATCTGCTGCTATCTATCCATGGACATCTGGTAGAGCTGGTAACTGTACTGCTACAGGTGCTTGTTTTGATTATCAATACCACTTGAACGGTGACATCGGTTTGTCTATGATCTATCAATGGGTTGCTTCAGGTGATACCGAATACTTCCAAGAAAAACATTTCCCTATCTACGATTCCGTTGCTACCTTGTACTCTAACTTGGTTGAAAGAAATGGTTCCTCTTGGACTTTGACTAACATGACTGATCCAGATGAATACGCCAATCATGTTGATGCCGGTGGTTTTACTATGCCATTGATTGCACAAACTTTGGAAAACGCTAACACCTTCAGACAACAATTCAACTTGGAACCTAACGATACCTGGACCGAAATTTCCGAAAATGTTTTGTTGTTAAGACAAAACAACGTCACCTTGGAATACACCTCTATGAATGGTACTGCAGTTGTTAAGCAAGCCGATGTTGTTTTAGTTACTTACCCATTGGCTTACGAATCTAACTACACTGCTGAAATGGCTTTGTCCGATTTGGATTACTACGCTAACAAACAATCTGCTGATGGTCCAGCTATGACTTGGGCTATTTTTTCTATTGTCGCCTCTGATGTTTCTCCATCTGGTTGTTCTGCTTGGACTTATCATCAATACTCTTACGATCCATACACCAGAGGTCCATTCTTCCAATTATCTGAACAAATGTTGGACAACGCCTCCATTAACGGTGGTACTCATCCAGCTTATCCATTTTTGACTGGTCATGGTGGTGCAAATCAAGTTGTCTTGTTCGGTTACTTAGGTTTGAGATTATTGCCAGAAGAAGGTATCTACATCACTCCAAATTTGCCACCACAAATCCCATACGTTAAGTACAGAACTTTCTATTGGAGAGGTTGGCCAATTGCTGCAGAATCTAATTATACTCATACCACCATCAGAAGAGATACCAAAACTGCTCCATTGTCTACTGCTGACGAAAGATTCAGAAATGCCACTATTCCAGTTCACGTTGGTTCTGATGAAGCTGAAACTCATACTTTACAACCTACTGGTTCCCCATTGATCATCGAAAACAGACAAATTGGTACAATCCCAACCATGCAAGGTAATCAAATTCAATGCCAACCTATCACTTCACCAGATGAACACAAAGCTGGTCAATTTCCAATTTCTGCTAACGATGGTGCAACTTCTACAAAATGGCAACCAGCTTCTTCTAACTTGTCCTCTATTACTGTTACCTTGTCCGATACTCAATTGGCTAATGCTGTTTCCGGTTTTCATTTTGATTGGGCTTCAGCTCCACCAGTTAATGCCTCTGTTATTTTCCATGAAGAAGTTATTGATAACCCAGCCTCCGTTTTTGCTTTTGGTACTCAAGATCAAGCTCAAGCTGAAGGTGACGAAAAGTATAGAGTTGTTTTGACCTTGACCGGTATTGAACCATCTACTATCTATACCGCCGAAGAAGAAAATCAAGTCAGAATTCCAGTTGGTAATACCACTACCACCCAATTGAAAGAAACTGTTAAGGCTTCTAAGTACGCCACCTTGTTGATTGCTGGTAATCAAGCTTTGTCTGGTGAACAAGAAGATGCTGGTGCAACAGTTGCAGAATGGGTTATTTTGTCTCAAGAAGGTGGTCAATCTCAATCAGCTGCTACTGCTCAAAGAAGAGGTATGAATGTTAGAGATAGAGCCTTGTTGGAAAGATTGAGAAGA

SEQ ID NO: 6 - protein, 1074 aa, Aspergillus flavus
MKLRNLAPWALLLTAVHGLPSQGTQNKHNPRVAKILKRHEGSSQKAKDSNNVYETKFDGVTWDEENWLLKTTTLDQGHYQSRGSVANGYLGINVASVGPFFELDEEVDGDVINGWPLYSRRQSFATIAGFFDSQPTTNGTNFPWLSQYGWDTAISGVPHWSGLILDLGDDVYLDSTVDDSTITDFQSTYDFKAGVLSWSYTWSPADKGSFEITYRLFANKLNITQAVVDMEIIPSVDANATVANVIDGYSAVRTDFVESGQDDGALFSAVRPWGISNVTAYIYTNLTGSANVDLSSRALVTGKPYVNTNESSVAQTVNVKFTAKEPVRITKFVGGASTDAFADPKQTAKEAASAALAAGYKNSLESHASEWANIMHENSVDRFTDPTTGKLPEDQHVIDSAVIAVTNIYYLLQNTVSQNAIAAVSNATVNETSFSVGGLTSDSYGGQVFWDADVWMQPGLVASHPEAAQGVTNYRVAKYQQAKENVKTAFTSSKNQTRFDPSAAIYPWTSGRAGNCTATGACFDYQYHLNGDIGLSMIYQWVASGDTEYFQEKHFPIYDSVATLYSNLVERNGSSWTLTNMTDPDEYANHVDAGGFTMPLIAQTLENANTFRQQFNLEPNDTWTEISENVLLLRQNNVTLEYTSMNGTAVVKQADVVLVTYPLAYESNYTAEMALSDLDYYANKQSADGPAMTWAIFSIVASDVSPSGCSAWTYHQYSYDPYTRGPFFQLSEQMLDNASINGGTHPAYPFLTGHGGANQVVLFGYLGLRLLPEEGIYITPNLPPQIPYVKYRTFYWRGWPIAAESNYTHTTIRRDTKTAPLSTADERFRNATIPVHVGSDEAETHTLQPTGSPLIIENRQIGTIPTMQGNQIQCQPITSPDEHKAGQFPISANDGATSTKWQPASSNLSSITVTLSDTQLANAVSGFHFDWASAPPVNASVIFHEEVIDNPASVFAFGTQDQAQAEGDEKYRVVLTLTGIEPSTIYTAEEENQVRIPVGNTTTTQLKETVKASKYATLLIAGNQALSGEQEDAGATVAEWVILSQEGGQSQSAATAQRRGMNVRDRALLERLRR

SEQ ID NO: 7 - DNA, 3126 bp, Fusarium oxysporum
atgttgtccttgcacttgaactacgttatcttgaccttgttgtcctctattactttggctacttctacccatgatcacgacagaatcaaaaagtgctatcaaagacacggtacttccagtgattctagaaaggcttctaacaacatctacaagacatctttcccaggtgttacttgggataatgataactggttgttgactaccaccaacttggatcaaggtcattatcaatctagaggttctgttgctaacggttatttgggtattaacgttgctgctgttggtccattctttgaaattgatgctgatgaagaaggtggtgttattaacggttggcctttgttttcaagaagacaaactttcgctaccattgccggtttttatgatgctcaacctaagaccaatggtacaaattttccatggttgttgcaatacggttacgaatccgttatttctggtgttccacattggggtggtttgattattgatttgggtgatgatgtttacttggatgccactgttgataacagaaccgttcataacttcacctctacctacgattttaaggctggtgttttggaatggtcttatacttgggaacctaaaggtaagggttcctaccaaatcaagtacagattattcgcccataagttgcacgttaatcaagccatagttgatttgaccatcgttccatctactgattctaaggctaaggttgttaacgttatcgatggttactctgccgttagatctgattttgttaagtccggtcaagacgaagatggtggtattttttctgctgttagaccagttggtattgctaacgttactgcttatatctacgctcaagttaacggttccaagtccttggatttgtctagaagaaaattggttcacggtaagccatacgttcataccaacgaatcttctattgctcaagccattccagttaagttttctgccggtgttccagttcatattactaagtatgttggtgctgcttcctctgatgcttttgaagatccagaaaagactgctaaagaagcctctcatagagctttggaagaaggttacgaaaagtctttgttgtcccatttgagagaatgggaatctgttatgccatccgattctgttgattcttacgcttttccagaaaacgataccttgccagatgacgaatatatcattgattccgccattatcactgtcaccaacacttattacttgttgcaaaacaccgttggtaagaatgctcaaaaagctgtttcaggtgctccagttaacatcgattctatttctgttggtggtttaacctctgattcatacgccggtttgattttttgggatgctgatttgtttatgcaaccaggtttgactacttctcatccagaagctgctcaaagaattaccaattacagagttgccaagtacgatcaagccaagaagaatattgctacttctttcgctggttcccaaaacaagaccaaattttctgaatcagctgctgtttacccatggacatctggtagatttggtaactgtactgctactggtccatgttgggattatgaataccatttgaacggtgacatcggtatttctttggttaatcaatgggttacctctggtgataccgacttcttcaaagaaactttgttgccaatctacgactccgttgctaatttgtttgccgatttgttgaagccaaatggttcctcttggactattaccaatatgaccgatccagatgaatacgccaatcatatagatgctggtggttttacaatggctttggcttctgaaactttgattcaagccaatcaaattagaagacaattcggtatgaccgaaaacaaaacccaagacgaaattgcttccgacgttttgttcattagagaaaacggtattaccttggaattcaccaccatgaatggttctgctattgtcaaacaagccgatgttgttttgatgtcttttccattgggttacaacgataactacaccgatcaaaacggtttggatgatttggattactacgccaacaaacaatctccagatggtcctgctatgacttgggctatctattctattgtcgctgatgaattgtctccatctggttgttctgcttacacttatgctcaatactcttacaagccatacactagaccaccattctaccaattgagtgaacaattggttgataacgccactgttaatggtggtactcatccagcttatccatttttgactggtcacggtggtgctaatcaagttactatttttggttacttaggtttgagattgatcccagaccaaggtttacatgttaatcctaatttgccaccacaaatcggttacttgaagtacagaactttctattggagaggttggccaatttctgcttggtctaattacactcataccactatctctagacatccaactactaagccattggatgttgctgattcaagatacgctaacaagggtattgctgtttatgctggtaaaatgggtgattctgcattgcatcatttgacctttgatgatccagttgtcatcaagaacagacaaattggtagtgtcaacaccgttcatggtaatttggctcaatgtagacctgtcaagtcctctaattcttacgaaccaggtcaatttccaattgctgcagttgatggtgcaacttctacaaaatggcaaccatctaaagctgccgatgtttcttctttgactgtttctttggctaagaaggatgttggttccaaagtcaagggtttttactttgattgggctgatgctccaccaatcaatgttacagttttgttccacaacaagactatcgatgatccaactaaggtttacggtacatcctctcatgattctggttatgatgttgtcgtcagtatcaaaaaggtcaagttgtccgatgcttacaacgctaagactgataatttggatgctgttgttatgcctactggtaacactactaatgtcactttgccagaaactgtcccattgtctagatatgctactttgttgatcgctggtaatcaagctttggataaggttgacttgaaagctggtaatggtactggtgctactgttgctgaatgggctattttacat

SEQ ID NO: 8 - protein, 1042 aa, Fusarium oxysporum
MLSLHLNYVILTLLSSITLATSTHDHDRIKKCYQRHGTSSDSRKASNNIYKTSFPGVTWDNDNWLLTTTNLDQGHYQSRGSVANGYLGINVAAVGPFFEIDADEEGGVINGWPLFSRRQTFATIAGFYDAQPKTNGTNFPWLLQYGYESVISGVPHWGGLIIDLGDDVYLDATVDNRTVHNFTSTYDFKAGVLEWSYTWEPKGKGSYQIKYRLFAHKLHVNQAIVDLTIVPSTDSKAKVVNVIDGYSAVRSDFVKSGQDEDGGIFSAVRPVGIANVTAYIYAQVNGSKSLDLSRRKLVHGKPYVHTNESSIAQAIPVKFSAGVPVHITKYVGAASSDAFEDPEKTAKEASHRALEEGYEKSLLSHLREWESVMPSDSVDSYAFPENDTLPDDEYIIDSAIITVTNTYYLLQNTVGKNAQKAVSGAPVNIDSISVGGLTSDSYAGLIFWDADLFMQPGLTTSHPEAAQRITNYRVAKYDQAKKNIATSFAGSQNKTKFSESAAVYPWTSGRFGNCTATGPCWDYEYHLNGDIGISLVNQWVTSGDTDFFKETLLPIYDSVANLFADLLKPNGSSWTITNMTDPDEYANHIDAGGFTMALASETLIQANQIRRQFGMTENKTQDEIASDVLFIRENGITLEFTTMNGSAIVKQADVVLMSFPLGYNDNYTDQNGLDDLDYYANKQSPDGPAMTWAIYSIVADELSPSGCSAYTYAQYSYKPYTRPPFYQLSEQLVDNATVNGGTHPAYPFLTGHGGANQVTIFGYLGLRLIPDQGLHVNPNLPPQIGYLKYRTFYWRGWPISAWSNYTHTTISRHPTTKPLDVADSRYANKGIAVYAGKMGDSALHHLTFDDPVVIKNRQIGSVNTVHGNLAQCRPVKSSNSYEPGQFPIAAVDGATSTKWQPSKAADVSSLTVSLAKKDVGSKVKGFYFDWADAPPINVTVLFHNKTIDDPTKVYGTSSHDSGYDVVVSIKKVKLSDAYNAKTDNLDAVVMPTGNTTNVTLPETVPLSRYATLLIAGNQALDKVDLKAGNGTGATVAEWAILH

SEQ ID NO: 9 - DNA, 3153 bp, Escovopsis weberi
atgagattgaacaatttggctgctgcctctttgttgttgagagctggtgcttgtttggaatctttccaagatagagtttctggttgcgttaacagacattcttctggttctcatccagctccatctaagaatgtttaccaaacttctttcgatggtgttacctgggatcaagataattggatgttgtctactaccgaattgcaacaaggtgcttttgaatctagagcttctgttgctaatggttacttgggtattaacgttgctggtgctggtccattttttgaattggattctgatgaaccaggtggtgttattaacggttggccattattttctagaagacaaaccttcgctaccattgctggtttttgggattctcaaccattgactgaaggtagaaatttcccatggttgtctcaatatggtggtgattctgctatttctggtgttccacattggggtggtttgttgttagatttgggtaacggtgaaattttggatgctgatgttgatgccgaaaccatctctgattttcaatctacctacgatttcaaggctggtgttatgacttggtcttacaaatggactccagcctctagaaaaaagactggtccaattggtatcacctacagattattcgctcacaagttgaacgttaatcaagccgttgttgacttggaaatcgttgctccaaaaggtgctcattctttgtctgctactgttgcttctgttttggatggttattctgctgttagaaccgattttgttggttctggtagagatggtgactctatctactcagctgttagaccagttggtattgcagatgttgaagcttatgtttacgcccaaatttctggttcacatggtgttgatatgtccagaaagagattggtttcttctcatggttctccatacgtcagatctaacgattcttcagttgttgaaaccgttccagtttctgtttctgctggtcaaactgttagagttactaagtttgttggtgccgcttcttctgatgcttttccagatccaagatctactgctagaactgctgttttagatgctgctaaagctggttttgatgccttgttgaaatctcatgctgctgaatgggctgaagttttgccagaagattctgttgattctttcgctgatccagaaactaacaagttgccacaagatgatatcttggttaccgatgctattatggctgttgttaacaccttctatttgttgcaaaacaccgttggtaagaacgctattgaagctgcttgtgatgctccattgaacgttgattctatttctgttggtggtttggcctctgattcttatgcaggtcaagttttttgggacgctgatttgtttatgggtccaggtttgtttacttcacatccagatgctgcacaaagaatctctaactacagagttaagttgtacgatcaagctaaggctaatgctcaaactggtttcacttcttctcaaaacgaaacccatattccagctgaagctgccgcttatgcttggatgtctggtagatttggtaattgcactgctacaggtccatgtttcgattacgaataccatttgaacggtgacatcggtttgtcctttgttaatcaatgggttgtttctggtgacaccgaatacttcaaagaaactttgttcccaatctacgattccatggctaccttgtatgcttctttgttgaagagaaatggttcctactggactttgactaacatgacagatccagatgaatacgccaacaacgttgatgctggtggttttactatgccattgattgctgaaatgttgagaaacgccaactccttcagacaacaattcggtttgccacaaaacgaaacttggaacgaaatggccgaaaatgttttgaccttgagagaaaacggtgtcactttggaattcactaccatgaacaattctgccgttgttaagcaagctgatgttatcatgttgaccttcccattgtcttacactgataactacaccaccgaaaactcattgaacgacttggattattacgccttggaacaatctccagatggtccagctatgacttatgcttacttctccattatcgccaatcaaatctctccatctggttgttctgcttacacttatgctcaaaatgctttcttgccatacttgagaggtccttggtttcaattgtctgaacaacaagttgataacgccactatcaatggtggtacacatccagcttatccatttttgactggtcatggtggtgctaatcaagttgttattttcggttatttgggtttgagattattgccagatgacatcttgcatatcaacccaaatttgccaccacaagttccttacgttagatacagagatttcttttggagaggtcatgctatttccgcttggtctaatgctactcatacaactttgtctagagctgcaagaactactccattggatactgctgatgctagatttgacacttctccaattactatctacgttggtgatgctgatcatccaactgtttacaaattaccaccaaagggttctgttgttgtcccaaatagacaagcaggttttgttgctaccaaagaaggtaatttggttcaatgcaagccagccatttctcatgatgatattatgcctggtcaattcccaattgctgctattgatggtgcttcttctactaagtggcaaccagcttctgctgataagttatcttctatgactgtctccttcgacaagagagatgttggttcattggtttccggtttttactttgaatgggcacaagctccaccagttaatgctacagttgttttccacgacgaattattgtctacctctggtaaaatcccatctggtaaaggtatagttgcccaattgtccaacattaagccatctaagccattcaatgttaccgctgctcaattggatattatcgctatgccagaatctaataccaccgaagttacattgaaacatccagttccagctactagatacgcctccttgtacattattggtaatcaaaagttgtccgctgctgatgtcgaagctaaaaatggtactggtgcaactgttgcagaatgggctattttgggtgaagaaaaagaaggttgcggtcctaagagattgatc

SEQ ID NO: 10 - protein, 1051 aa, Escovopsis weberi
MRLNNLAAASLLLRAGACLESFQDRVSGCVNRHSSGSHPAPSKNVYQTSFDGVTWDQDNWMLSTTELQQGAFESRASVANGYLGINVAGAGPFFELDSDEPGGVINGWPLFSRRQTFATIAGFWDSQPLTEGRNFPWLSQYGGDSAISGVPHWGGLLLDLGNGEILDADVDAETISDFQSTYDFKAGVMTWSYKWTPASRKKTGPIGITYRLFAHKLNVNQAVVDLEIVAPKGAHSLSATVASVLDGYSAVRTDFVGSGRDGDSIYSAVRPVGIADVEAYVYAQISGSHGVDMSRKRLVSSHGSPYVRSNDSSVVETVPVSVSAGQTVRVTKFVGAASSDAFPDPRSTARTAVLDAAKAGFDALLKSHAAEWAEVLPEDSVDSFADPETNKLPQDDILVTDAIMAVVNTFYLLQNTVGKNAIEAACDAPLNVDSISVGGLASDSYAGQVFWDADLFMGPGLFTSHPDAAQRISNYRVKLYDQAKANAQTGFTSSQNETHIPAEAAAYAWMSGRFGNCTATGPCFDYEYHLNGDIGLSFVNQWVVSGDTEYFKETLFPIYDSMATLYASLLKRNGSYWTLTNMTDPDEYANNVDAGGFTMPLIAEMLRNANSFRQQFGLPQNETWNEMAENVLTLRENGVTLEFTTMNNSAVVKQADVIMLTFPLSYTDNYTTENSLNDLDYYALEQSPDGPAMTYAYFSIIANQISPSGCSAYTYAQNAFLPYLRGPWFQLSEQQVDNATINGGTHPAYPFLTGHGGANQVVIFGYLGLRLLPDDILHINPNLPPQVPYVRYRDFFWRGHAISAWSNATHTTLSRAARTTPLDTADARFDTSPITIYVGDADHPTVYKLPPKGSVVVPNRQAGFVATKEGNLVQCKPAISHDDIMPGQFPIAAIDGASSTKWQPASADKLSSMTVSFDKRDVGSLVSGFYFEWAQAPPVNATVVFHDELLSTSGKIPSGKGIVAQLSNIKPSKPFNVTAAQLDIIAMPESNTTEVTLKHPVPATRYASLYIIGNQKLSAADVEAKNGTGATVAEWAILGEEKEGCGPKRLI

SEQ ID NO: 11 - DNA, 3162 bp, Microsporum gypseum
atgggtaagccaaacattagattggttgctggtatagtctggttgttgttgattattgctgctgttactgctgaaaccgatgctgaaagaaatgctggtgtttttgctagaaactccgctttgaaaaaaggttcctctggttctgaacaaccagtttatgctactagattcaagggtgttacttgggatgttgctaattggagattgactactaccgaattggatcaaggtcactatcaatccagaggttctattgctaatggttatttgggtattaacgttgctgcagttggtccattctttgaattggatgttccagtttctggtgatgttattaacggttggccagttttctctagaagacaaactttcgctaccatctccgatttctactcattccaacaatctattaacgccactaacttcccatggttgaacaaatatggtggtgatttgatttccggtgttccacattggtctggtttgattttggatttgggtgatggtaacttcttggatgctactgttcaaaactccaccatttctaacttcacctctaccttggatatgaagggtggtattttgacttggcaatatacttggtccccagaaaaacataacggtacttacgatatcttctatcaattggttgcccacaagttgcatgttaatcaagctttggttagaatggaaatcaccccatctaaggatggtaacgtttctgttgttaacgttatcgatggttactctgctgttagaactgattttaagggttctggtcaagatggtggtgctatctatacttctgttaaccctgaaggtatctctaacgttactgcttttatctacgctgaaatgtctggtactgaaggtgttaacttgtcctcttcttctttggttaacgacaaaccatacttgcataccaacggttctactattgctcaatctgttaacgtcaaattgagagctggtcaaactaccaagatcgataagtttgttggtgctgctactactgaccaattcaagaacccaagacaagctgctaaagatgcttctgctagagctttgagaactggttatgaagaatccttgaaaacccatattgctgaatggactactgttttcccatctgattctactgaagattacaccattccaggtaaaaagtggttgccattggatcatcatattatcgaagcctccattgtctccgttgttaatccatattacttgttgcaatccaccgcttctcataatgctttgactgctgttaagaatgccccattgaatagaggtagtattgctgttggtggtttgacttctgattcttacggtggtttaatcttctgggatgctgatatttggatgcaaccaggtttggttgttgcttttccagaagcttcacaaatcttctccaactacagagttgataagtatggtcaagccttgagaaacgctcaaactcaagatttgtcctccaagaaaaagacctacttttctccagatgctgccgtttatccatggacatctggtagatttggtaaatgtactgctactggtccatgcttcgattaccaatatcatttgaatggtgacatcggtatgcaaatcgttaacaattgggttactactggtgacaccgaatacttcaagtctaagttgtttccagtctacaactctattgccaccttcttttctcaattggtcgaaaagaatggtactcaatggactgttaccaacatgactgatccagatgaattcgctaacttggttgacggtggtggttatacaatgccattgattgctaccactttgaagtacgccaatcaattcagagaaatgttcggtttgggtgctaatcaaacttggtctgaaattgcccaaaacgtccaagtttctagagatccagcttctcaaattaccttggaatacactactatgaacggttccactcaagttaagcaagctgatattgtcttgaacaccttcccattgagatacaccgaagattatactcatgataacgccttgagagacttggattattacgctgctaaacaatcaccaaatggtccagctatgacttacgctattttttccatcgttgccaacgaagtttctccatcaggttgttctgcttatacttacggtcaatactcattctccccatatgttagagccccatttttccaattctccgaacaagttgttgatgactggtctatcaatggtggtactcatccagcttatccatttttgactggtaacggtggtgcaaatcaagttgctgtttttggttacttaggtttgagattggtttccgatggtatcttgcatttgaatccaaatttgccaccacaaatcccacacattagatacagaactttctattggcatggttggccatttgaagcctctgctaattatacccaaaccactattcaaagagctaccaacagaagaccattggcttcagctgatccaaaatttgctaatgctccaatcactgttcacgttggtccagaatctaatattaccgtttattccttgccaccatccggtcaattagttattccaaatagaagatccggttccatcaacaccttggaaggtaacttagttcaatgtcaacctgtctactctccaaacgaatttgctccaggtcaatttcctatttctgctgttgatggtgctgcatctactaagtggcaacctagaagagcttcttcaacatcttctttgactgtttctttgccagatgatgcttcctctgcttctatttcaggttttgcttttgattgggctcaagctccaccaatttcagctaaagttgttttacacgatgaaccattgccaccagttatggatgctgaagatgatgcaggtaatggtttttctcatgctactccaccaggttctgttactgtttgggaaactccagaagttccacaatctcatccatacgatccaattaccatcgacttgaacatgattatgacctacaagggtaacactaccaacattactttgccatctgctgttccagctactaagtttgctaccttgttgatcagaggtaatcaagccttaggtccagctgaagttaaggctggtaatggtactggtgctacagttgcagaatggtctattttgagatctact

SEQ ID NO: 12 - protein, 1054 aa, Microsporum gypseum
MGKPNIRLVAGIVWLLLIIAAVTAETDAERNAGVFARNSALKKGSSGSEQPVYATRFKGVTWDVANWRLTTTELDQGHYQSRGSIANGYLGINVAAVGPFFELDVPVSGDVINGWPVFSRRQTFATISDFYSFQQSINATNFPWLNKYGGDLISGVPHWSGLILDLGDGNFLDATVQNSTISNFTSTLDMKGGILTWQYTWSPEKHNGTYDIFYQLVAHKLHVNQALVRMEITPSKDGNVSVVNVIDGYSAVRTDFKGSGQDGGAIYTSVNPEGISNVTAFIYAEMSGTEGVNLSSSSLVNDKPYLHTNGSTIAQSVNVKLRAGQTTKIDKFVGAATTDQFKNPRQAAKDASARALRTGYEESLKTHIAEWTTVFPSDSTEDYTIPGKKWLPLDHHIIEASIVSVVNPYYLLQSTASHNALTAVKNAPLNRGSIAVGGLTSDSYGGLIFWDADIWMQPGLVVAFPEASQIFSNYRVDKYGQALRNAQTQDLSSKKKTYFSPDAAVYPWTSGRFGKCTATGPCFDYQYHLNGDIGMQIVNNWVTTGDTEYFKSKLFPVYNSIATFFSQLVEKNGTQWTVTNMTDPDEFANLVDGGGYTMPLIATTLKYANQFREMFGLGANQTWSEIAQNVQVSRDPASQITLEYTTMNGSTQVKQADIVLNTFPLRYTEDYTHDNALRDLDYYAAKQSPNGPAMTYAIFSIVANEVSPSGCSAYTYGQYSFSPYVRAPFFQFSEQVVDDWSINGGTHPAYPFLTGNGGANQVAVFGYLGLRLVSDGILHLNPNLPPQIPHIRYRTFYWHGWPFEASANYTQTTIQRATNRRPLASADPKFANAPITVHVGPESNITVYSLPPSGQLVIPNRRSGSINTLEGNLVQCQPVYSPNEFAPGQFPISAVDGAASTKWQPRRASSTSSLTVSLPDDASSASISGFAFDWAQAPPISAKVVLHDEPLPPVMDAEDDAGNGFSHATPPGSVTVWETPEVPQSHPYDPITIDLNMIMTYKGNTTNITLPSAVPATKFATLLIRGNQALGPAEVKAGNGTGATVAEWSILRST

SEQ ID NO: 13 - DNA, 3222 bp, Aspergillus clavatus
atgttgtctaccaatttggctacttgggcttctttgttggcttgtttgccagctactttggcttttcaaactaacaaccatgctagagtcaccagatctttgaaaagacatgctggtcatggtcatactccaccaactgatacaaattcctctaacatctacgaaaccagattcccaggtgttacttgggataatgataattgggttttggctaccactaccttggatcaaggtcattatcaatctagaggttctgttgctaacggttatttgggtattaacgttgcttctgttggtccattcttcgaattggatactccagtttctggtgatgttattaacggttggcctttgttctctagaagacaatcttttgctaccattgccggtttctttgatttccaacctactaccaatggttctaacttcccatggttgaatcaatacggtggtgaatctgttatttccggtgttccacattggtctggtttggttttggatttgggtgatgatacttatttggatgctgccgttgataacgaaaccatttctggttttcaatccgcctacgattttaagtctggtgttttgtcttggtcttacacttggactccaactgatgataagggttctttcaacatcacctacagattattcgccaacaagttgcatatcaatcaagccgttgttgacatggaaattaccccatctcaagaatctcaagctaccgttgttaacgttatcgatggttattctgctgttagaaccgatttcgttgaatccggtgaagatgatggtgctattttttcagctgttagaccatggggtattgctaatgttactgcttacgtttacgctaacttgaccgcttctaagaatgttgatttggcttctcatactttggttgccgataagccatatatccataccaacgaatcttcagttgctcaagcagttagagttaacttcagagctaacgaaactgtcagaatcactaagtttgttggtgctgcttcttctgatgcttttccagatccacaaaaaactgctaagcaagctgtttctgctgctttgggtgctggttatatggaatctttacaatcccatgttgctgaatgggccgatattttgttggatggttcagttgattctttcgttgatccagttactggtaagttgccagatgatgaacatatcgttaactcccaagttattgctgttgccaacacttactacttgttgcaaaacactgttggtaagaacgctactactgctgtttcagatgctccagttaatgttgactctattagtgttggtggtttgacctctgattcttacgctggtcaagttttctgggatgctgatgtttggatgcaaccaggtttggttgcttctcatccagaagctgctcaaagaattaccaatttcagagtcgtccaataccaacaagctttggaaaatgttaacactgctttcaccggttccaagaatcaaacttctttttctccatctgctgctatctatccatggacatctggtagatttggtaactgtactggtactggtccatgttgggattatcaataccatttgaacggtgacatcggtttgtctttgatgtaccaatggattacctctggtgataccaaggttttcagagaacaacatttcccaatctacgattccattgctaccttgtactctaacttggtcgaaagaaacggttcttcttggactttgactaacatgactgatccagatgaatacgccaaccatattgatgctggtggttttactatgccattgatctctgaaactttgggttacgctaataccttcagaaagcaattcggtcacgaacaaaacgaaacctggtctaagattgccgaaaacgtcttggttatcagagaaaacgatgttaccttggaatacactaccatgaatggtactactgttgttaagcaagccgatgttgttttggttacttacccattggtttacgacaacaactatacctccgaatactccttgaacgatttggatttctacgctaacaagcaatctccagatggtcctgctatgacttgggctatttttgctattactgccaacgatgtttcaccatctggttgttctgcttacacttaccatcaaaactcttacgacccatatatgagagccccatttttccaattgtccgaacaaactattgatgacgcttctatcaatggtggtactcatccagcttatccatttttgacaggtcatggtggtgctaatcaagttgtcttgttcggttacttaggtttgagattattgccagacgatgccattcatatcgatccaaatttgccaccacaaattccaaatgttgcttacagaactttctactggcatggttggccaatttctgcttcatctaatagaacccataccaccatatctagagctaccaaaattgctccattggatacagctgatcctagatttgctaacgtttccattccagttttggttggttacgatacaaacgctactgcttatcatttgcctccatctggtccattgactgttagaaatagacaaatcggtttgaacaacaccatccctggtaacattattcaatgcagaccagtttactcccctgatgattatgctccaggtcaatttcctattgctgctgttgatggtgcaacttctacaaaatggcaaccagctactactaatacttctgctttgacagttactttgcctgatgccgaagttaattctgttgtttcaggtttccatttcgattggtggcaagctccacctgttaatgctactgttattttccatgacgaaaccttggaagatcctgttacagctttatcttcttctcatggtaacccacaatacactgttattactaccttgaccaacatcgaattgtcccaaccttataacgctgaatcctctgatttgaacaaagttgctatgccaactggtaacactaccgatgttcaattgtcatctactgttcatgctgctagatacgccactttgttaatttcaggttctcaaggtgatggtgatgccggtgctacagtagcagaatgggctatattgggtcaagaaaaagaatcttccggtcacgataatggtaagagaagattggatgttagatctgctgcagctttgtctggttctttggatgatagaagagctagaagattcaccgct

SEQ ID NO: 14 - protein, 1074 aa, Aspergillus clavatus
MLSTNLATWASLLACLPATLAFQTNNHARVTRSLKRHAGHGHTPPTDTNSSNIYETRFPGVTWDNDNWVLATTTLDQGHYQSRGSVANGYLGINVASVGPFFELDTPVSGDVINGWPLFSRRQSFATIAGFFDFQPTTNGSNFPWLNQYGGESVISGVPHWSGLVLDLGDDTYLDAAVDNETISGFQSAYDFKSGVLSWSYTWTPTDDKGSFNITYRLFANKLHINQAVVDMEITPSQESQATVVNVIDGYSAVRTDFVESGEDDGAIFSAVRPWGIANVTAYVYANLTASKNVDLASHTLVADKPYIHTNESSVAQAVRVNFRANETVRITKFVGAASSDAFPDPQKTAKQAVSAALGAGYMESLQSHVAEWADILLDGSVDSFVDPVTGKLPDDEHIVNSQVIAVANTYYLLQNTVGKNATTAVSDAPVNVDSISVGGLTSDSYAGQVFWDADVWMQPGLVASHPEAAQRITNFRVVQYQQALENVNTAFTGSKNQTSFSPSAAIYPWTSGRFGNCTGTGPCWDYQYHLNGDIGLSLMYQWITSGDTKVFREQHFPIYDSIATLYSNLVERNGSSWTLTNMTDPDEYANHIDAGGFTMPLISETLGYANTFRKQFGHEQNETWSKIAENVLVIRENDVTLEYTTMNGTTVVKQADVVLVTYPLVYDNNYTSEYSLNDLDFYANKQSPDGPAMTWAIFAITANDVSPSGCSAYTYHQNSYDPYMRAPFFQLSEQTIDDASINGGTHPAYPFLTGHGGANQVVLFGYLGLRLLPDDAIHIDPNLPPQIPNVAYRTFYWHGWPISASSNRTHTTISRATKIAPLDTADPRFANVSIPVLVGYDTNATAYHLPPSGPLTVRNRQIGLNNTIPGNIIQCRPVYSPDDYAPGQFPIAAVDGATSTKWQPATTNTSALTVTLPDAEVNSVVSGFHFDWWQAPPVNATVIFHDETLEDPVTALSSSHGNPQYTVITTLTNIELSQPYNAESSDLNKVAMPTGNTTDVQLSSTVHAARYATLLISGSQGDGDAGATVAEWAILGQEKESSGHDNGKRRLDVRSAAALSGSLDDRRARRFTA

SEQ ID NO: 15 - DNA, 2433 bp, Metarhizium anisopliae
atgagagccactttgattcaagctgctgttgttgctgctactttgggtttgtgttctgctgttcaagcagctaatggtaaagatagagttgctaagtgtttggccagatactctggtcaagacagaggtagaaatagaactactgtttacaagaccgatttcccaggtgttacttgggatgatgataattggttgttgtctactaccaccttggaacaaggtagatatcaatctagaggttctgttgctaatggttacttcggtatttcagttgcttctgttggtccattctttgaattggatgctgaagatgaaggtggtgatgttattaacggttggcctttgttttccagaagacaatcttttgctaccattgctggtttttggaatgctcaaccagaaactaacggtactaatttcggttggttgttacaatacggttacgaatctgttatctccggtgttccacattggtctggtttggttttggatttgggtaatggtgtttacttggattccaccgttgataacaagaccattaccaacttcagatccacctacgattttaaggctggtgttttgtcttggtcttatacttggtcaccatctgctggtaacaatggttcttacgatatcagatacttgatgttcaccaacaagttgcatatctcccaagcagttgttgatttggaaatcgttccatctgttgatgctaacgctactgttgttaatgttttggatggttactctgccgttagaaccgattttgttcaatctggtgaagatgctggtgctatctattcagctgttagaccaactggtattgctaacgttacagcttacatctacgctaacatgactggttctgatgatgttgatattggtagaaagaccttggtcagtaacaagccttacatcagaaagaacgaatcctctattgctcaagctgttccagttactttttcagctggtaaggctgttagaatcactaagtatgttggtgctgcttctggtgatgcttttgatgatccacaacaagtcgctaaaaatgctgcttcatctgctttgtctcaaggtttctacaagtccttgagatcccatgttcaagaatgggatgacgttatgccagatcattctgttgattcttatgccgatccagataatggtactttgccacaagattcctacattatcgattccgctattattgctgttgccaacacttactacttgttgcaatctacagttggtccaaacgctcaatctttggttaaggatgctccagttaacgttgattctattagtgttggtggtttggtcagtgattcttacgctggtttgattttttgggatgccgatttgtttatgcaaccaggtttggttgtttcccatccacaatctgctgaaagaatcaccaattacagagtcaacaaatacggtcaagctaaggctaatgctcaaacttcttacacctcctctcaaaacaaaaccgttttttccaaagatgctgcagcttttccatggacatctggtagatttggtaactgtactgctactggtccatgttgggattatcaataccatttgaatggtgatatcggtatctccttcgtcaatcaattggttgctacaggtgatacaagatacttcaacgaatctttgttcccagtctacgattctattgccactttgttctctaatttgttggccccaaatggttcatcttggactgttaagaatatgaccgatcctgatgaatacgccaatcatgttgatgccggtggttatacaatgccattgattgctgaaacattgcaaactgctaacaccttcagagaacaattcggtttggaaaagaatgctacctgggattctatggctaccaatgtcttgtttttgagagaaaacggtgttaccttggaattcactactatgaatggttctgccgttgttaagcaagccgatgttattttgaataccttcccattgtcttacaccactaactacactacccaagaatccttgaacgatttggattactacgccaacaaacaatctccagatggtcctgctatgacttgggcttttttttctattatcgccaacgacatttctccatctggttgttctgcttacacttactctcaatactcttacaagccatatgctagagccccattttaccaattgtctgaacaattgatcgataacgccactatcaatggtggtactcatccagcttatccatttttgactggtcatggtggtgctaatcaagttaatgtattcggttacttaggtttgagattattgccagatgataccttgcatatcaacccaaatttgccaccacaattgtcccatttgagatacagaactttctattggagaggttggccatttgctgcatcttctaatgctactcataccacc

SEQ ID NO: 16 - protein, 811 aa, Metarhizium anisopliae
MRATLIQAAVVAATLGLCSAVQAANGKDRVAKCLARYSGQDRGRNRTTVYKTDFPGVTWDDDNWLLSTTTLEQGRYQSRGSVANGYFGISVASVGPFFELDAEDEGGDVINGWPLFSRRQSFATIAGFWNAQPETNGTNFGWLLQYGYESVISGVPHWSGLVLDLGNGVYLDSTVDNKTITNFRSTYDFKAGVLSWSYTWSPSAGNNGSYDIRYLMFTNKLHISQAVVDLEIVPSVDANATVVNVLDGYSAVRTDFVQSGEDAGAIYSAVRPTGIANVTAYIYANMTGSDDVDIGRKTLVSNKPYIRKNESSIAQAVPVTFSAGKAVRITKYVGAASGDAFDDPQQVAKNAASSALSQGFYKSLRSHVQEWDDVMPDHSVDSYADPDNGTLPQDSYIIDSAIIAVANTYYLLQSTVGPNAQSLVKDAPVNVDSISVGGLVSDSYAGLIFWDADLFMQPGLVVSHPQSAERITNYRVNKYGQAKANAQTSYTSSQNKTVFSKDAAAFPWTSGRFGNCTATGPCWDYQYHLNGDIGISFVNQLVATGDTRYFNESLFPVYDSIATLFSNLLAPNGSSWTVKNMTDPDEYANHVDAGGYTMPLIAETLQTANTFREQFGLEKNATWDSMATNVLFLRENGVTLEFTTMNGSAVVKQADVILNTFPLSYTTNYTTQESLNDLDYYANKQSPDGPAMTWAFFSIIANDISPSGCSAYTYSQYSYKPYARAPFYQLSEQLIDNATINGGTHPAYPFLTGHGGANQVNVFGYLGLRLLPDDTLHINPNLPPQLSHLRYRTFYWRGWPFAASSNATHTT

SEQ ID NO: 17 - DNA, 2889 bp, Ogataea parapolymorpha
atggctcaaccagattacttcgatgatcaaaccgaatcctattacttgcaagacgaaagagttttgggtactaccaagttcaatcaattgaacaagtacacctaccaaccatacgtttccaatggttacattggttccagaattccaaacttgggtttcggtttttcctacgaccaaaacgaaaacttgacctcctctgatttgtctaatggttggcctttgtttaacccaagatacgctggttcttttattgccggtttttttgatgctcaacctaacactactggtgtcaatttcccagaattgagagaaaacggttacgaatccgttatttctgctgttccacaatggactgctttacaattggctgctactttgaatggtgaaacctatgttttggatccttctactgctaacacttcctctgctcatgttactgattacagacaagaattgagaatggctaccggtactgtttctacagcttatacttggttgggtgctgttactgttaacattactgttatggcccacagagatttcgaaaccttgggtttagttcaattggaagttgctccagtttctggtgctgctccattgaaattggatgttgttgatgtcttggatttcgcctctactcaaagatgtgttttggaatccattggttacgatgatgccggtattttcattaccgttcaaccagaaggtgttgcttacaaacatgcctcattatactccagattgaacgttaacgcttcctgcattaacgaaactttagctgctgctttccataaggttaccaacactgtttctttggttttggaacacccattgtctgttactaagtacgttggtgttgtttccgatgatttgttgggtacaaattcttctgatgcaactttggctgcagctaaaagaactgctttggatgctgctaaatattcctggccatctttaagaaccatgcatgataatgcttgggctgatgtttggggtgatgttgctgttgaagttgaaaatgaaccatacttgactttggcagctgaagcttctatctaccatttgtttgctaacaccagatcctctgctagaaatttgacagctgctttgtctgttggtggtttgtcatctgattcttacggtggtttggttttttgggatgctgatttgtggatgattccagctttgttgccaattgctccagaaacttctgttgctttgaactcctacagatattacttacacgaacaagccgttagaaatgctgctgctaattcttattcaggtgctgtttatccatggacctctggtagatttggtaactgtactggtactggtccatgcattaactacgaatatcatttgaacggtgccatctgttactctgtttggaaagcttatttgtccggtgccattaacgatgaacatttggaacaatacggttggccagttttgagagatgctgctgatttttttgccgattacgttagatacaacgacaccttgcaaaagtacactactcataacttgaccgatccagatgaatacgctaactttaaggataacgctgcttacaccgctgttgttatttctcaagtaatgaagtgggctgatagagttgctagacatttgggtaaaccatctaactctacccaattgaagatcatggaaaacatgtacttgccacaatccagagataacatcactttggaatacgacaccatgaactcctctgttttgattaagcaagccgatgttgttttgatcccttacatcgatgatgaagatggtgctttggctcaaaatttcggttatgatgaagttagagccaccaacgatttgtcctactattcattgcatcaatcctctcaaggtccagctatgacttttccagtttttgctgctgtttcccaaaagttgaatgattacggttgtggttctcaaacctaccactacaaatctgttgctccatttttgagattcccattcgctcaaatgtccgaacaaaacaacgataactacgatgctaatggtggtactcatccagcttttccattcaatactgctcatggtggtttagtccaatcttacttttttggtttgaccggtatcagattctcttacgctgttactccagaacacagattgcaaagagtcttgcattttgatccagtcgaattgccattattctccggtgatttgaagatctctggttttaagtacttgaatcaatccttggaaatcgtcatcggtgaaactaacggtactattagacatagaggtactgccgaatccatcttggtttatgttgatgatagaaacgctgcagctggttactatactttggaaccaggtactgaattgaccgttccagtttatgttaagcaattcaacactccaggttctttgactgaatgtcaagctttagctcattcattgactccaggtagagatggtgatgtcattatgtccattatcgatggtgataactctactacttggcaagctgaaaacaagaatggtaatgctgcagtcttgttggaattcgaaactactgaaacttttaacgctggtgctatcgtttggggtaatagaccagctgctaacttctctttatctgttgttgctgaacctttggataccactggtacagatgttgttatcgacgaaactaagttggttagagttttgaccgaccacgttgttcaaattgcttctccttataacgcctccgataccgaagttagaattgcagaacctaattccaccattttcgctttgccacaagaatacactgctcaatacgttttgttggaagtctacggtacattggatactgatgattctacttacggtgcttcagttgctgaattgggtttgttttaccac

SEQ ID NO: 18 - protein, 963 aa, Ogataea parapolymorpha
MAQPDYFDDQTESYYLQDERVLGTTKFNQLNKYTYQPYVSNGYIGSRIPNLGFGFSYDQNENLTSSDLSNGWPLFNPRYAGSFIAGFFDAQPNTTGVNFPELRENGYESVISAVPQWTALQLAATLNGETYVLDPSTANTSSAHVTDYRQELRMATGTVSTAYTWLGAVTVNITVMAHRDFETLGLVQLEVAPVSGAAPLKLDVVDVLDFASTQRCVLESIGYDDAGIFITVQPEGVAYKHASLYSRLNVNASCINETLAAAFHKVTNTVSLVLEHPLSVTKYVGVVSDDLLGTNSSDATLAAAKRTALDAAKYSWPSLRTMHDNAWADVWGDVAVEVENEPYLTLAAEASIYHLFANTRSSARNLTAALSVGGLSSDSYGGLVFWDADLWMIPALLPIAPETSVALNSYRYYLHEQAVRNAAANSYSGAVYPWTSGRFGNCTGTGPCINYEYHLNGAICYSVWKAYLSGAINDEHLEQYGWPVLRDAADFFADYVRYNDTLQKYTTHNLTDPDEYANFKDNAAYTAVVISQVMKWADRVARHLGKPSNSTQLKIMENMYLPQSRDNITLEYDTMNSSVLIKQADVVLIPYIDDEDGALAQNFGYDEVRATNDLSYYSLHQSSQGPAMTFPVFAAVSQKLNDYGCGSQTYHYKSVAPFLRFPFAQMSEQNNDNYDANGGTHPAFPFNTAHGGLVQSYFFGLTGIRFSYAVTPEHRLQRVLHFDPVELPLFSGDLKISGFKYLNQSLEIVIGETNGTIRHRGTAESILVYVDDRNAAAGYYTLEPGTELTVPVYVKQFNTPGSLTECQALAHSLTPGRDGDVIMSIIDGDNSTTWQAENKNGNAAVLLEFETTETFNAGAIVWGNRPAANFSLSVVAEPLDTTGTDVVIDETKLVRVLTDHVVQIASPYNASDTEVRIAEPNSTIFALPQEYTAQYVLLEVYGTLDTDDSTYGASVAELGLFYH

SEQ ID NO: 19 - DNA, 3471 bp, Kluyveromyces marxianus
atgatcatcatcccattggtcgttttggttttcactgttttggctccagtttacttctacgttactaagccagaatcttccacccattctttgtttccagaattagctccagctagaatttcttggccatttgctggtacttgtgcttcttcatctggtggtgaagaagatccattatattgtccagatgcttacagaaaggcctccgaaaaaatgtacgatttgttgaaggataacgaatacgccttctacgacgaaacttctgaaactttgggtaacttgttgttgtccgaaaacaccttttctagacaaccatacgttgccaatggttacattggttctagaattccaaatgtcggtttcggttttgcttacgatgctattaacatctgggttaacgattctgctattccaggtgctttgaacaatggttggccattgagaaatcaaagatacgctggttctttcgtcagtgacttttactccttgcaagaaaagttgaactctaccaactttgccgaattggataaggatggttactccaccgttatttcttcaattccagattggaccgacttgtccattatgattcatagaggtccaggtgaaaacaacgtcgaatatatcaatccaaccgatgtcaagttggataagatcaccgattacatgcaaaacttgtctatgagagatggtatcgttaccaccaagttcgtttacgataacaacttgttcgttactaccagaaccttggctcatagatcaatctatccattgggtatcgttgacatggaaatcgaattattgccacaagctaccgaaaacggtttacatgaagctagtgttgaattggaaatctgcgatactttcaacttcactacttcccatagaaccgttttggctgattttggtcatgacaagaagaacgaaggtatctacatgatcgttgaaccagaaaacgttccatactctaacgcttctatgttctcctacttcgatatcccatctagagatgaatacactgttgctaagactaacgactctgtctctcaatgtactagaagagttttgaccaccgactctagagaaaactctactttcatcgttagaaagtttaccggtatcgtttcctccgaatatgataacaacaacccagaacacatgtccaacttggaaagagctactgctgttgttatggaaaacaagggtgattacaagaacttgttgaagatgcatagagatcactggaagagattatacgctgatgcctctattgaaatcccatctgatggtttgttggaaatgaccgctaaatcctctatctaccatttgttggctaactccagatcccataacgtttctcaatctagaggtttgccagttcctccatctggtttgtcatctgattcttatggtggtatggttttctgggatgctgatgtttggatgttgccagctttgttgccattttttccagaaattgccaagcaaatgtctgcctacagaaatgcttctttggctcaagctaaagaaaacgccaaaaagtatggtttacaaggtgctatttttccatggacctctggtagatttgctaactgtacttctactggtccatgcgttgattacgaataccacattaacgttgatatcgccttgtcctccttgtacatctatatgtcaggtgaagaagatgaagaaaagtccgaagaatacttgagatacactacctggccattcattgaaaatgctgctaagatgttcaccgactacgttaagtggaacgacactttacaacaatacaccactcataacttgaccgatccagatgaattcgctaaccatgttgataatggtgctttcactaacgccggtattaagtctattatgggttgggctcatgatattgccaatcatttgggtttggatccagatcctaaatggactgaaattgccgaaaagatccatatcccaatctccgataccaacattactttggaatatactggtatgaactcctccgttgatattaagcaagctgatgttgttttgatgacctacccattgggttacttcactgaaacttctcaacctagaaacgccatcaaggatatctactactactccgaaagacaatctgcttctggtccagctatgacttatccagtttttgttgctgcttcagcctccttgttgaattctggttcttcttctcaatcctacttgtacaaatccgttgtcccttatttgagatctccattcgctcaattctccgaacaatctgatgataacttcttgactaacggtttgactcaaccagcttttccatttttgactgctaatggtggttacttgcaatccattttgtttggtttgaccggtttgagatactcctacgaagttgataaggatactggtaagatgcacagattattgaagttcaacccaatctctttgccaatgtttccaggtggtatcagaatcaacaacttcaagtatatgggtcaagtcttggacatcttgttgaccgataatgaaggtatcatcaagcacaagaagggtaacaagtccattttgatcaagatcccagatagaggtgatatccctgatgttaagcctgatgaatatacccaaatcaacggtacttctgtcaatgttaagagagctgttccatcaggtgaatcctaccatacaattgaaccaggtactgttttcaagaccccattatacaaccctaagagaaacatggctaacaacatcgtcgaatctaagagagccactaatattactgttggtgttccaggtgatgttgctgtttctgcaattgatggtaacaactacactcactggcaaccagctaacaaaaaacaaccaggtagaatcttgatcgacatgggtaatggtactgctaacgaaatcaagtccggtaagattttgtggggtaatagaccagctaagtccttctctttgtctatcttgccacaattcgatcaaattacccaaaacatgacctccgttttgtctcaaccatcttcacataattgctccaatgatgatggttgggattctaactgcaagtaccaagaggaagaagaaaacattgacgctgccattaaggatgtttttgaatggtatggtatggacttgcaaagtgtcatcgaaaactatccagaattgtccaatgtctccatgggtttcattaagttggttgatcattacaacgtcaccccatcttacccttggaagaatgttaattccaccagaatcgaattgaccttgggtaacgaaactaacttcgttgtcgattactctaaggtcccagaattgaacttgaacaacaatttgggtgtcgacttacaatccaaagacactagatggagaaagccaagatttgttgtcttgactgttttcgatacctacgatgatgatgacgaagttaagggtgctaccatcaaagaattgtctttgttcgacaat

SEQ ID NO: 20 - protein, 1157 aa, Kluyveromyces marxianus
MIIIPLVVLVFTVLAPVYFYVTKPESSTHSLFPELAPARISWPFAGTCASSSGGEEDPLYCPDAYRKASEKMYDLLKDNEYAFYDETSETLGNLLLSENTFSRQPYVANGYIGSRIPNVGFGFAYDAINIWVNDSAIPGALNNGWPLRNQRYAGSFVSDFYSLQEKLNSTNFAELDKDGYSTVISSIPDWTDLSIMIHRGPGENNVEYINPTDVKLDKITDYMQNLSMRDGIVTTKFVYDNNLFVTTRTLAHRSIYPLGIVDMEIELLPQATENGLHEASVELEICDTFNFTTSHRTVLADFGHDKKNEGIYMIVEPENVPYSNASMFSYFDIPSRDEYTVAKTNDSVSQCTRRVLTTDSRENSTFIVRKFTGIVSSEYDNNNPEHMSNLERATAVVMENKGDYKNLLKMHRDHWKRLYADASIEIPSDGLLEMTAKSSIYHLLANSRSHNVSQSRGLPVPPSGLSSDSYGGMVFWDADVWMLPALLPFFPEIAKQMSAYRNASLAQAKENAKKYGLQGAIFPWTSGRFANCTSTGPCVDYEYHINVDIALSSLYIYMSGEEDEEKSEEYLRYTTWPFIENAAKMFTDYVKWNDTLQQYTTHNLTDPDEFANHVDNGAFTNAGIKSIMGWAHDIANHLGLDPDPKWTEIAEKIHIPISDTNITLEYTGMNSSVDIKQADVVLMTYPLGYFTETSQPRNAIKDIYYYSERQSASGPAMTYPVFVAASASLLNSGSSSQSYLYKSVVPYLRSPFAQFSEQSDDNFLTNGLTQPAFPFLTANGGYLQSILFGLTGLRYSYEVDKDTGKMHRLLKFNPISLPMFPGGIRINNFKYMGQVLDILLTDNEGIIKHKKGNKSILIKIPDRGDIPDVKPDEYTQINGTSVNVKRAVPSGESYHTIEPGTVFKTPLYNPKRNMANNIVESKRATNITVGVPGDVAVSAIDGNNYTHWQPANKKQPGRILIDMGNGTANEIKSGKILWGNRPAKSFSLSILPQFDQITQNMTSVLSQPSSHNCSNDDGWDSNCKYQEEEENIDAAIKDVFEWYGMDLQSVIENYPELSNVSMGFIKLVDHYNVTPSYPWKNVNSTRIELTLGNETNFVVDYSKVPELNLNNNLGVDLQSKDTRWRKPRFVVLTVFDTYDDDDEVKGATIKELSLFDN

SEQ ID NO: 21 - DNA, 3117 bp, Komagataella phaffii
atgccatacggttctatctacaactccagaattccaaaaaaaccaccaccaacttctcaaaccagagaaatgttgaacagagttttgttggttgccttgtcttgcgttgttttcttccatttggttactactttcccagtcggtacttcttctgattccttgcaaattagaaacttgttgtcccacaacttcaccagagctaatatctctgaaggtttatcttctggtgctacctacttcgttgatgaagatactgaaacctactacgacaaagaattgaaggttttgagaaccaccagattcccaagatacaacaactatcaattgcaaccatacgttgccaacggttacattggttctagaattcctagagttggttccggttttacttacgatacttctgataacaagacctccgaaaacttgaaaaatggttggcctttgttcaacaagagatactctggtgcttttattgccggtttctttaactctcaacctaccgttccagaaactaacttcgaagaattggaaaaggacggttacgaatccattattgcctctattccacaatggacctcattggaattgactgttaatgtcaacggtactaatcaaaccttgaaggccgatgatgttgatatcacccatatttccgattactcccaacaattgtctttgttggatggtatcgttactaccaactatacttggttgggtttggtcaacgtttccatttctgttttggctcacagagacatagtttctttgggttttgtctccttggaattgtcctcccaaaagaacattactgtttccgttaccgatatcttggatttcgctacttctaccagatgctcttatttggattctggtgtcaacgaacaatccattttcatgaaggttcaaccatctaacgttccaactaacgctactatctactcctctttgatgtcctctaattccacctcatctttattgaagcaaaatcaaaccgtttcccaaactttgagagtcaacttgtctaagaatcaagctgcctcattccaaaagtatgttggtgttgtttccgatgactacttggactctattgaaacaaacttgacctcctaccaattcgctagagaaactgctaaattcgctgaaatcaaaggtagatcctggatcttgaagtctcacaaagaagcttggaacgaattattgaacggtaaatccatcgttttccacgacaacgattttttgactttggcctctgattcttccatctatcatttgatggctaacaccagatctgaagctaatggtggtacttctgctttgggtgtttctggtttgtcatctgattcatatggtggtatggttttctgggatactgatttttggatgttgccatccgttcaagctttttcaccaagacatgctgtttctttgtccaagttcagagatcatactcatgatcaagctaagaagaacgcccaaactagagatatgaatggtgctgtttatccatggacctctggtagatttggtaactgtacttctactggtccatgctacgattacgaataccacattaacattgatatcgccttcatgttttggaagttgtatttgggtggtgccatcgatgatgattacatgaaggaattcggttacccaattatcgaagatgttgcctcttttttcgttgactacgtcgattacaattctaccttggataagtacaccaccagaaacttgactgatccagatgaatatgccgaattcaagaacaatgctgctttcactaacgtcggtatctctcaattgatgaagtgggctttgatcttgggtaaacatttgaaggttggtaacgaaagatcctacgataagtgggaagatatcatgaccaaaatgtacttgccagttaaccatgctggtgatgttactttggaatacactggtatgaacaactccatcgaagttaagcaagctgatgttgtcttgatctcttacccattggatgacgaagatggtgctttacaagaatacttcgattacgacgaagatagagccatctccgatgttagatattactccgataagcaaacagatgaaggtccagctatgactttctctgtttactctgctgttaacgccaagttcaacaaagaaggttgttcctctcaaacttacttgttgaagtctgtcgaaccatacttcagatttccattcggtcaaatgtctgaacaatctaccgatcaatacgatacaaacggtggtactcatccagcttttccatttttgactggtcatggtgcctttttacaatcctctatctatggtttgaccggtttgagattctcctacatctataacgatactgacaagtccatcaagagaagattggcttttgatccattgcaattgccatgtttgccaggtggtttctctattaacggtttcgtctacatgaatcaaactttggatatcaccgttaacgacacctatgctactattgctcatagaggtaatgccactaccatcaatgtttacgttgactctagaaacgaaatgggtggtaaagaacataagatccaaccaggtaagtccttgtccattccattataccaaaccgaacaaaacatcccaggttccttcattgaatgtaccgttaagaatgttactgccttgcaaccaggtgttgttggtgatccaattcaagctgttgctgatggtgataactctaccatttggaagatcgaatctagagaagaacctacccatttgatcttcgatttgggtgatgaattggacattgaaggtggtttggttgtttggggtacttacccagctgaatctttttcagtttcagtcttgagagatttcaactccaccaactacagagtcatcaacaacgttgaaaactacgacttgatctacgaatctggtaatgttacagcttcctctccattcgatgaatcccatatcaaaaaggtccaaatcttgccacataactgcactaactttaccttctctgaattgaccgcttccagatacgttttgtttgaattcactgatgtcttgggttacccacaagattattcttatggtgctcaagttgcagaagtcgtcttgtat

SEQ ID NO: 22 - protein, 1039 aa, Komagataella phaffii
MPYGSIYNSRIPKKPPPTSQTREMLNRVLLVALSCVVFFHLVTTFPVGTSSDSLQIRNLLSHNFTRANISEGLSSGATYFVDEDTETYYDKELKVLRTTRFPRYNNYQLQPYVANGYIGSRIPRVGSGFTYDTSDNKTSENLKNGWPLFNKRYSGAFIAGFFNSQPTVPETNFEELEKDGYESIIASIPQWTSLELTVNVNGTNQTLKADDVDITHISDYSQQLSLLDGIVTTNYTWLGLVNVSISVLAHRDIVSLGFVSLELSSQKNITVSVTDILDFATSTRCSYLDSGVNEQSIFMKVQPSNVPTNATIYSSLMSSNSTSSLLKQNQTVSQTLRVNLSKNQAASFQKYVGVVSDDYLDSIETNLTSYQFARETAKFAEIKGRSWILKSHKEAWNELLNGKSIVFHDNDFLTLASDSSIYHLMANTRSEANGGTSALGVSGLSSDSYGGMVFWDTDFWMLPSVQAFSPRHAVSLSKFRDHTHDQAKKNAQTRDMNGAVYPWTSGRFGNCTSTGPCYDYEYHINIDIAFMFWKLYLGGAIDDDYMKEFGYPIIEDVASFFVDYVDYNSTLDKYTTRNLTDPDEYAEFKNNAAFTNVGISQLMKWALILGKHLKVGNERSYDKWEDIMTKMYLPVNHAGDVTLEYTGMNNSIEVKQADVVLISYPLDDEDGALQEYFDYDEDRAISDVRYYSDKQTDEGPAMTFSVYSAVNAKFNKEGCSSQTYLLKSVEPYFRFPFGQMSEQSTDQYDTNGGTHPAFPFLTGHGAFLQSSIYGLTGLRFSYIYNDTDKSIKRRLAFDPLQLPCLPGGFSINGFVYMNQTLDITVNDTYATIAHRGNATTINVYVDSRNEMGGKEHKIQPGKSLSIPLYQTEQNIPGSFIECTVKNVTALQPGVVGDPIQAVADGDNSTIWKIESREEPTHLIFDLGDELDIEGGLVVWGTYPAESFSVSVLRDFNSTNYRVINNVENYDLIYESGNVTASSPFDESHIKKVQILPHNCTNFTFSELTASRYVLFEFTDVLGYPQDYSYGAQVAEVVLY

SEQ ID NO: 23 - DNA, 3540 bp, Ashbya gossypii
atggctgatactgcttctttgccaccacaaagagattctgctttgggtatgcatggtccacatggtggtttgtatatgccagttgctcaaggtccattgcaagctcatgcttctccaagattggtttctgttagaatggtcttgtcctctattactgctttggctttggttgctgttgttactgttttgggtactgctcaaccagctagaccaactgctccattggctgctgctgatgaacaattttgggttgcacaacatagatccgcctctaagcaattatatcaattggttcacggttccgaattgtccttttatgatgaaggtagagatgtcttgggtactaccgaattatctagaaacatgtactccagacaaccatacgttgctaatggttacattggttctagagttccaaatgtcggttttggttatgctgcagatgaagaaaacatttggactgatgcttcagttccaggtgctttgaacaatggttggccattgagaaatccaagatacgctggttctttcgtttccgatttttactccttgcaagccagattgaattctaccaatttcccagaattggacgaagaaggttactctactgttattgcctctattccagaatggaccgatttgagagttagagctgatggtgctgaattgggtgctgaaactgttgctttggaagatatgggtggttacgttcaaaacatgtctttggcagatggtgttgttaccactgaatatgtttggagaggtttgtctgttagagctactgttgctgctcatagatctgaatatccattgggtttggttcaattggaagttgctttgtgtggtgatactgaacctagagaagttgaagtcagagatgtattgaacttcaccacttctcatagaaccgttttgagagaagctggtcatgatgaagatggtatctacatgagagttgaaccagaaaacgttccatattctgaagctgccttgtactctgttttcgaagttagaggtggtgaaggttctgttcaaccagaaagagctgctgccggtgctacagttgcacaatgggttagagttagattgactgctgcacaacctagagttgttgttagaaagtatgttggtgtcgtttcctctgaatacaatactgctggtggttctaatttggaagcagctagagctgccgctttggctcattatggtgcttttgatggtgcattggtttcacatagagctgcttggtctgcattatatggtaacgcctctattgaaatcccttccgatttcttgttggaattggctgcaaaatcctccatgtttcatatgttggctaacactagagcccataacgtttctgctactagaggtttgccagttccagttactggtttatcttctgattcttacggtggtatggttttttgggattctgatgtttggatgttgccaggtttgttgccattttttccagatattgccagagaaatctccaactacagaaatgctactcatgctcaagctgttgcaaatgctagacattacaattattccggtgccttgtatccatggacatctggtagatatgctaactgtacttctactggtccatgcgttgattacgaataccatatcaacattgatatcgccatgtcctcattgtccatctatatgaatggtgcagatggtattggtgaagattacttgagatataccacttggcctttgttgagagatgcagctttgtttttcaccgaatacgtcagatacaacgaaaccttggatgcttacactactcataacttgactgatccagatgaattcgccaacttcattgataacggtgcttttactaatgccggtattaagatcttgttgagatgggctattgatgtcggtactcatttggaagaaccagttgataccaagtggcaagaaatctctgataagattcatatcccaacctccgaaactaacatcactttggaatacactggtatgaacgctaccgttgatattaagcaagctgatgttttgttgatggtctaccctttgggttacattaccgatgaatccattttgaacaacgccatccaaaacttgtactactactccgaaagacaatctgcttctggtccagctatgacttatccagtttttgctgctgcagctgctactttgttgaatcatggttcatcttcccaatcctacttgtacaaagctgttgttccatacttgagagccccatttttccaattctctgaacaatccgatgacaactttttgactaacggtttgacacaaccagctttcccatttttgacagctaatggtggttacttgcaatccttgttgtttggtttgactggtttgagatactcctacactgttaacccagaaactaagaagatggaaagattattgaagttctccccagtcagaatgccattattgccaggtggtattagaatcaacaactttaagtacttgggtcaagtcttggacatttccatcgatgatcataacgctaccattgctcacaagcaaggtaatactccaatccatattaaggtcccagacagatctatcttgagagatagagatgttccagtctacaaaggttcagctttacaagccagagatgttatcccataccatgaattgtccaactctaactactttactgtcaacccaggtgaaactttgacattgccagtttacgaaccagaattgaacattcaaggtaacatcgtcgaaggtagacaaattaccaatttgactcaaggtgttcctggtgatgttccaatctctattttggacggtaacaactacactcattggcaaccatttgacaagtctgaaagagctttgttgttgatcgatttgggttccgaagaagaatacgaaattactaccggtaagattttgtggggtgctagaccagctaagaacttttccatttctatcttgccaaactccaagcacatcaccgaaattttgacaaagttgaccgctatgatggatggtagaaacactgatttggtttcttgctctaagtgccatgctgtttcatcttctcaacatttgttgggtggtttggctaacgttactgattctaaaggtttggctgctatcgatggtgaaactgtcgatatgggtatcagagaaattttcagatggaacttgttcgacttgccaaccatctcttctattataccagaagctgccaacatttccgaatctttcgttacagttttggaaaactaccaagtcactccatccgaaccatattacgaagaagttgtcagaaagtcccaaatcgtcattttgccatctaacgaaaccgatttctgcattgattatgctgctgttccaaagttgaacccaacttacactgctgttaatttgtccgctgatgataccaattggagaaagactagattcgttatcgttgctgtcgaaggttcttacgatgatgatgacgatcaaaaaggtggtactatcaaagaaatcgctttgatggttgctccaaaaaat

SEQ ID NO: 24 - protein, 1180 aa, Ashbya gossypii
MADTASLPPQRDSALGMHGPHGGLYMPVAQGPLQAHASPRLVSVRMVLSSITALALVAVVTVLGTAQPARPTAPLAAADEQFWVAQHRSASKQLYQLVHGSELSFYDEGRDVLGTTELSRNMYSRQPYVANGYIGSRVPNVGFGYAADEENIWTDASVPGALNNGWPLRNPRYAGSFVSDFYSLQARLNSTNFPELDEEGYSTVIASIPEWTDLRVRADGAELGAETVALEDMGGYVQNMSLADGVVTTEYVWRGLSVRATVAAHRSEYPLGLVQLEVALCGDTEPREVEVRDVLNFTTSHRTVLREAGHDEDGIYMRVEPENVPYSEAALYSVFEVRGGEGSVQPERAAAGATVAQWVRVRLTAAQPRVVVRKYVGVVSSEYNTAGGSNLEAARAAALAHYGAFDGALVSHRAAWSALYGNASIEIPSDFLLELAAKSSMFHMLANTRAHNVSATRGLPVPVTGLSSDSYGGMVFWDSDVWMLPGLLPFFPDIAREISNYRNATHAQAVANARHYNYSGALYPWTSGRYANCTSTGPCVDYEYHINIDIAMSSLSIYMNGADGIGEDYLRYTTWPLLRDAALFFTEYVRYNETLDAYTTHNLTDPDEFANFIDNGAFTNAGIKILLRWAIDVGTHLEEPVDTKWQEISDKIHIPTSETNITLEYTGMNATVDIKQADVLLMVYPLGYITDESILNNAIQNLYYYSERQSASGPAMTYPVFAAAAATLLNHGSSSQSYLYKAVVPYLRAPFFQFSEQSDDNFLTNGLTQPAFPFLTANGGYLQSLLFGLTGLRYSYTVNPETKKMERLLKFSPVRMPLLPGGIRINNFKYLGQVLDISIDDHNATIAHKQGNTPIHIKVPDRSILRDRDVPVYKGSALQARDVIPYHELSNSNYFTVNPGETLTLPVYEPELNIQGNIVEGRQITNLTQGVPGDVPISILDGNNYTHWQPFDKSERALLLIDLGSEEEYEITTGKILWGARPAKNFSISILPNSKHITEILTKLTAMMDGRNTDLVSCSKCHAVSSSQHLLGGLANVTDSKGLAAIDGETVDMGIREIFRWNLFDLPTISSIIPEAANISESFVTVLENYQVTPSEPYYEEVVRKSQIVILPSNETDFCIDYAAVPKLNPTYTAVNLSADDTNWRKTRFVIVAVEGSYDDDDDQKGGTIKEIALMVAPKN

SEQ ID NO: 25 - DNA, 2076 bp, Neurospora crassa
atggtcagtagatttttgggtgctactgttccattggctgctgctattttgccaggtgctagagcattatatgttaacggttctgttactgctccatgcgattctccaatctactgttatggtgaattattgcaccaagtcgaattggctagaccattctctgattctaagacctttgttgatatgccaaccatcaagccagttgatgaagttttggaagctttctctaagttgaccttgccattgtctaacaactccgaattgcatgaattcttgtctacttactttggtccagctggtggtgaattggaagctgttccaactgatcaattgcatgtttctccaactttcttggacaacgtttccgatgatgttatcaagcaattcgttgactccgttattaacatttggccagatttgaccagaaagtatgttggtgccggtgaattgtgtactggttgtgctgattctttcatcccagttaacagaacttttgttgttgctggtggtagattcagagaaccatattactgggattctttctggatcttggaaggtttgttgagaactggtggtgctttcactgaaatctccaagaacattatcgaaaactttttggacttggtcgaacaaatcggttttgttccaaatggtgctagattgtactacttggatagatctcaaccaccattattgacccaaatggttagaatctacgttgaacataccaacgacacctccattttggaaagagctgttcctgttttgaagaaagaatgggaatggtggactaccaacagaactgttgaagttactgctgatggtaagacctactcattgcaaagataccacgttgacaacaatcaacctagaccagaatcttacagagaagattacattaccgccaacaacaactcttactatgctacctctggtatcatctacccagaaactactccattgaacgatactcaaaaggctttgttgtacgctaatttggcttctggtgctgaatctggttgggattattcttctagatggttgaagaatccaggtgatgctgctagagatgtttactttccattgagatccttgaacgtcttggaaatcgttccagttgatttgaactccatcttgtaccaaaacgaagttaccatcggtaagttcttggctcaacaaggttctaaagatgaagctgaagaatgggctaaaaaggccgaagaaagatctgaagctatgtacaagttgatgtggaactctactttgtggtcctacttcgattacaacttgacctcttcttctcaaaacatctacgttccagctgatccacaagtttttccatttgaacaaccatctggtactccagaaggttaccaagttttgttctccgtcaatcaaatgtttccattctggactggtgctgctccagatcaattgaaaggtaatccattagctgttaagttggccttcgaaagaatcaagaacttgttggataacaaggccggtggtattccagctactaattttgttactggtcaacaatgggatgaacctaatgtttggccaccattgatgcatgttttgatggatggtttattgaacactccagctacctttggtgaagatgatccagcttatcaagaaactcaaaccttggctttgagattggctcaaagatacgttgattctactttctgtacttggtatgctactggtggttctacttctgaaactccaaaattgcaaggtttgggttctgatttgaagggtatcatgttcgaaaagtactccgataactctacaaacgttgctggttcaggtggtgaatatgaagttgttgaaggttttggttggaccaacggtgttttgatttgggctgctgataagtttggtgacaagttgaaaagaccagattgcggtgatattactccagctcaagttggtaaaagagccgatattactatggaaaagagagccgttgaattggacgtttttgatgctaagttcaccaagaagtttgccagaaagggtaaattggaaaagttgaaggccaagttcaaaagaagagctgccatt

SEQ ID NO: 26 - protein, 692 aa, Neurospora crassa
MVSRFLGATVPLAAAILPGARALYVNGSVTAPCDSPIYCYGELLHQVELARPFSDSKTFVDMPTIKPVDEVLEAFSKLTLPLSNNSELHEFLSTYFGPAGGELEAVPTDQLHVSPTFLDNVSDDVIKQFVDSVINIWPDLTRKYVGAGELCTGCADSFIPVNRTFVVAGGRFREPYYWDSFWILEGLLRTGGAFTEISKNIIENFLDLVEQIGFVPNGARLYYLDRSQPPLLTQMVRIYVEHTNDTSILERAVPVLKKEWEWWTTNRTVEVTADGKTYSLQRYHVDNNQPRPESYREDYITANNNSYYATSGIIYPETTPLNDTQKALLYANLASGAESGWDYSSRWLKNPGDAARDVYFPLRSLNVLEIVPVDLNSILYQNEVTIGKFLAQQGSKDEAEEWAKKAEERSEAMYKLMWNSTLWSYFDYNLTSSSQNIYVPADPQVFPFEQPSGTPEGYQVLFSVNQMFPFWTGAAPDQLKGNPLAVKLAFERIKNLLDNKAGGIPATNFVTGQQWDEPNVWPPLMHVLMDGLLNTPATFGEDDPAYQETQTLALRLAQRYVDSTFCTWYATGGSTSETPKLQGLGSDLKGIMFEKYSDNSTNVAGSGGEYEVVEGFGWTNGVLIWAADKFGDKLKRPDCGDITPAQVGKRADITMEKRAVELDVFDAKFTKKFARKGKLEKLKAKFKRRAAI

SEQ ID NO: 27 - DNA, 2085 bp, Thielavia terrestris
atggctccaagatcttttgttgctgctgctgcattggctggtttgatttcttctgcttctgccttgtatatcaacggttctgttactgctccatgcgattctccattatattgccatggtgaaattttgaaggccatcgaattggctcatccattcactgattctaagacctttgttgatatgccaaccatcagaccattggatgaagttattgctgctttcaacagattgtcccaaccattgtctaacaactctgaattgaatgctttcttggctgctaattttgctccagctggtggtgaattggaagctgttccaagagatcaattgcatactgaaccatctttcttgaacaagttggatgacaccgtcatcaaagaattcgttgccaaggttattgatatctggcctgatttgactagaagatatggtggtccaggtaactgtactgcttgtgctaattctttcatcccagtcaacagaactttcgttgttgctggtggtagattcagagaaccatattattgggattcctactggatcttggaaggtttgttgagaactggtggtgctttcactgaaatctccaagaacatcatcgaaaatttcttggacttcgtcgaaaccatcggttttattccaaatggtgctagaatctactacttggacagatctcaaccaccattattggctagaatggttagatcctacgttgattacactaacgacacctccattttggatagagctttgccattattgatcaaagaacacgaattctggtccaccaatagatccgtttctattaaggctccaaacggtaagacttacaccttgaacagatattacgtcaacaacaatcaacctagaccagaatccttcagagaagattacattactgccaacaacggttcctattatgctgcttctggtattatctacccagttaacactccattgaacgatactgaaaaggctgaattatacgctaacttggcttctggtgctgaaactggttgggattattctactagatggttgaagaatccaaacgatgctgctaaggatatctacttcccattgagatctttgaacgttagaggtactgttccagttgacttgaactctatcttgtacgaaaacgaagtcatcatctcccaatacttgaaaagagccggtaacaattctgaagctgaaagatgggcttacgctgcttctcaaagatcagaagctatgtttgaattgatgtggaacgctactcattggtcctacttcgattacaacttgacctctaactcccaaagaatctttgttccagtagatgatgatgctactgctgctgaaagagctggtgctccaagaggtcaacaagttttgtttaacatcggtcaattctacccattctggactggtgctgctccagcacaattgaaaaacaatccattggctgttcaacaagcttacgctagagttgctagaatgttggacgaaaatgccggtggtattccagctactaattttgttactggtcaacaatgggatcaacctaatgtttggccaccattgcaacatgttttgatggaaggtttattgaacactccaccaacttttggtgatgctgatccagcttatcaatctgttagagctttagctttgagattggcccaaagatacttggattctactttctgtacttggtatgctactggtggttctacttctcaaactccacaattgcaaggtgttgctccaggtgctgaaggtattatgttcgaaaagtacgctgataactctaccaatgttgcaggttcaggtggtgaatatgaagttgttgaaggttttggttggtccaacggtgttttgatttgggctgctgatgtttttggtgcccaattgaaaagaccagattgtggtaacattaccgctgctcatacttctggttctggtgcacaaaaaagatcaggtggttctttagctagaagagccgttgaattggatccatgggatgctgcttggacaaaaatgtttggtagatccgctttgaagaagagagaagacgttagaaagagatggttgttggctgct

SEQ ID NO: 28 - protein, 695 aa, Thielavia terrestris
MAPRSFVAAAALAGLISSASALYINGSVTAPCDSPLYCHGEILKAIELAHPFTDSKTFVDMPTIRPLDEVIAAFNRLSQPLSNNSELNAFLAANFAPAGGELEAVPRDQLHTEPSFLNKLDDTVIKEFVAKVIDIWPDLTRRYGGPGNCTACANSFIPVNRTFVVAGGRFREPYYWDSYWILEGLLRTGGAFTEISKNIIENFLDFVETIGFIPNGARIYYLDRSQPPLLARMVRSYVDYTNDTSILDRALPLLIKEHEFWSTNRSVSIKAPNGKTYTLNRYYVNNNQPRPESFREDYITANNGSYYAASGIIYPVNTPLNDTEKAELYANLASGAETGWDYSTRWLKNPNDAAKDIYFPLRSLNVRGTVPVDLNSILYENEVIISQYLKRAGNNSEAERWAYAASQRSEAMFELMWNATHWSYFDYNLTSNSQRIFVPVDDDATAAERAGAPRGQQVLFNIGQFYPFWTGAAPAQLKNNPLAVQQAYARVARMLDENAGGIPATNFVTGQQWDQPNVWPPLQHVLMEGLLNTPPTFGDADPAYQSVRALALRLAQRYLDSTFCTWYATGGSTSQTPQLQGVAPGAEGIMFEKYADNSTNVAGSGGEYEVVEGFGWSNGVLIWAADVFGAQLKRPDCGNITAAHTSGSGAQKRSGGSLARRAVELDPWDAAWTKMFGRSALKKREDVRKRWLLAA

SEQ ID NO: 29 - DNA, 3219 bp, Aspergillus lentulus
atgttgtccaagaatttggctacctgggtttctttgttggcttgtttgccagctactattggttctccaaacaacaacgatagaatcgccagatctttgaaaagacatggtggtcatggtcataagcaagctgatacaaattcctcccatgtttacaagactagattcccaggtgttacttgggatgatgatcattggttgttgtctactactaccttggatcaaggtcactatcaatccagaggttctattgctaatggttatttgggtatcaacgttgcttctgttggtccatttttcgaattggatgttccagttggtggtgatgttattaacggttggccattatactctagaagacaaacttttgctaccattgccggtttcttcgattatcaacctactaccaatggttctaacttcccatggttgaatcaatacggtggtgaatctgttatttccggtattccacattggtccggtttgattttggatttgggtgatggtaattacttggatgctaccgttgataacaagaccattaccgatttcagatccacctacgattttaagtctggtgttttgtcttggtcttacacttggacaccaagatgtaacaagggttctttcgatatcacctacagattattcgcccataagttgcatgttaatcaagccgttgttgacatggaaattactccatctcaaggttctgaagccactgttgttaatgttatcgatggttactctgccgttagaaccgattttgttgaatctggtcaagattctggtgctttgttttctgctgttagaccatggggtatttctaacgttactgcttacgtctataccaacttgactgcttctgctggtgttgatttgtcatcaagagctttggttaacgataagccatacgttcactctaacgaatcttcagttgctcaagctgttaacgtcaaattcagagctaacgaaaccgttagaatcaccaaatttgttggtgctgcttcttctgatgcttttccaaatcctcaacaaaccgctaaacaagctgtttctgctgctatgggtgctggttatatgggttctttacaatctcatgttgctgaatgggcctccattttgttggatggttctgttgattctttcgttgatccagctactggtaaattgccagatgatgaccatatcttgaactcccaaattattgctgttgccaacacttactacttggtccaaaacactgttggtaagaacgctattaaggctgtttcaggtgctccagttaatgtcaactctattagtgttggtggtttgacctctgattcttatgctggtttggttttttgggatgctgatgtttggatgcaaccaggtttggttgcttctcatccagaagctgctcaatcagttactaattacagaactaagttgtacccacaagccttggaaaacatcaatactgctttcacctcctctaagaatcaaacctctttttctccatctgctgcaatctatccatggacatctggtagatttggtaactgtactggtactggtccatgttgggattatcaataccatttgaacggtgacatcggtttgtctttgatgtatcaatgggttgcttccggtgataccaagacttttagagaacaacatttcccaatctacgattctgttgccaccgtttactctaacttagtccaaagaaatggttcctcttggactttgactaacatgactgatccagatgaatacgccaatcatgttgatggtggtggttttactatgccattgatttctgaaaccttgtcctacgctaactccttcagaaagcaatttggtttggaacaaaacgaaacctggaccgaaatctccgaaaatgttttggttatcagagaaaacggtgtcaccatggaatacactactatgaatggtactaccgttgtcaagcaagcagatgttgttttagttacctacccattggtttacgacaacaactatactgctcaatacgccttgaacgatttggattattacgccaacaaacaatctccagatggtcctgctatgacttgggctatttttgctattactgccaacgatgtttcaccatctggttgttctgcttacacttaccatcaagattcctacgatccatatatgagagccccattttaccaattgtccgaacaaatgattgatgacgcctctatcaatggtggtactcatccagcttatccatttttgacaggtcatggtggtgctaatcaagttgttttgatgggttacttaggtttgagattattgccagacgacgccattcatattgatccaaatttgccaccacaagtctccaacgttaagtacagaactttttattggagaggttggccaatttccgcttcttctaatagaactcataccaccatatctagagctgctaatattgctccattggatactgctgattctagatttgctaacgccaccatttcagttttggttggtgatccatctaattccactgcttatagattaccagctacaggtccattggtcgttccaaatagacaaattggtttcaacaacaccatccctggtaacatggttcaatgtagaccagtttattccccacatgattatgctccaggtcaatttccaattgctgctgttgacggtgctacttctactaagtggagaccagcaacagctaatatgtcatctttgactgttaccttggccgatgttgaaatcaactctaaggtttctggtttccatttcgattggtggcaagctccacctgttaatgctactgttattttccatgacgaaatgttggaagatccagttgctgcagtttcttctgctcatggtaattctagatacaaggttgttaccaccttgaccaacatcaaacaatcccaaccatacaacgctgaatctaccgattacaacgaagttgttatggctacaggtaacactaccgatgttaacttgtctcaaactgttcacacatctagatacgccaccttgttgattagtggtaatcaagctggtggtaaagaaggtgctacagtagcagaatgggcaattttgggtgaatctaaaggttcttcttcaggtcacggtaacaacaagagaagattagatgttagagctgccgctgctttgtctggtggtttaaatgatagaagatacagacaattcaatgct

SEQ ID NO: 30 - protein, 1073 aa, Aspergillus lentulus
MLSKNLATWVSLLACLPATIGSPNNNDRIARSLKRHGGHGHKQADTNSSHVYKTRFPGVTWDDDHWLLSTTTLDQGHYQSRGSIANGYLGINVASVGPFFELDVPVGGDVINGWPLYSRRQTFATIAGFFDYQPTTNGSNFPWLNQYGGESVISGIPHWSGLILDLGDGNYLDATVDNKTITDFRSTYDFKSGVLSWSYTWTPRCNKGSFDITYRLFAHKLHVNQAVVDMEITPSQGSEATVVNVIDGYSAVRTDFVESGQDSGALFSAVRPWGISNVTAYVYTNLTASAGVDLSSRALVNDKPYVHSNESSVAQAVNVKFRANETVRITKFVGAASSDAFPNPQQTAKQAVSAAMGAGYMGSLQSHVAEWASILLDGSVDSFVDPATGKLPDDDHILNSQIIAVANTYYLVQNTVGKNAIKAVSGAPVNVNSISVGGLTSDSYAGLVFWDADVWMQPGLVASHPEAAQSVTNYRTKLYPQALENINTAFTSSKNQTSFSPSAAIYPWTSGRFGNCTGTGPCWDYQYHLNGDIGLSLMYQWVASGDTKTFREQHFPIYDSVATVYSNLVQRNGSSWTLTNMTDPDEYANHVDGGGFTMPLISETLSYANSFRKQFGLEQNETWTEISENVLVIRENGVTMEYTTMNGTTVVKQADVVLVTYPLVYDNNYTAQYALNDLDYYANKQSPDGPAMTWAIFAITANDVSPSGCSAYTYHQDSYDPYMRAPFYQLSEQMIDDASINGGTHPAYPFLTGHGGANQVVLMGYLGLRLLPDDAIHIDPNLPPQVSNVKYRTFYWRGWPISASSNRTHTTISRAANIAPLDTADSRFANATISVLVGDPSNSTAYRLPATGPLVVPNRQIGFNNTIPGNMVQCRPVYSPHDYAPGQFPIAAVDGATSTKWRPATANMSSLTVTLADVEINSKVSGFHFDWWQAPPVNATVIFHDEMLEDPVAAVSSAHGNSRYKVVTTLTNIKQSQPYNAESTDYNEVVMATGNTTDVNLSQTVHTSRYATLLISGNQAGGKEGATVAEWAILGESKGSSSGHGNNKRRLDVRAAAALSGGLNDRRYRQFNA

SEQ ID NO: 31 - DNA, 3198 bp, Aspergillus ochraceoroseus
atgagagtcacccaattattggttaagggttccggtttacaatccaagaacttgagattgtctggttgcatgagaagacatggtggtggtcatggtattgaaaaaactttggttggttccaacaacacctaccaaactgtttttccaggtgtttcttgggatgatgatcattggttgttgactactactactccagatccaggtcattatcaatctagaggttctgttgctaacggttacattggtatctccgtttcttctattggtccattcttcgaattggatatgccagttgatggtgatgttatttctggttggcctttgttctctagaagacaatcttttgctaccattgccggtttctatgattaccaacctactaccaatggttctaacttcccatggatcaatcaatacggtggtgaatctgttatttcaggtgtaccacattggtccggtttgattattgatttgggtgacgaaacctacttggattccactgttgacaatcaaactatcaccggtttctcttctacctacgattttaaagctggtatgttgtcttggtcttacacttggactccagctgctggtgataagggttcttacaaaattacctacagaatcttcgccaacaagttgaacgttaatcaagccgttgttgacatggaaatcatcccatctattgattccgaagccatcatcgttaatgttttggatggttatgctgccgttagaaccgattttgtttcttctggtcaagatgatggtgctatctattctgctgttagaccatggggtatcgaaaatgttactgcttacatctacgctaacattaccggttctgatgctgttcatttgtcctcaagaagaatcgttactggtaaggcttacgtcaacatcaacgaatcttcaattgctcaagctgtcgatgttaagttctctgcttctgaaaaggttagaatcaccaagtttgttggtgctgcttctactgatgctttttctgatccacaacaaactgctaagcaagctgtttctcaagctttgactgctggttacttgagatgcttgcaatctcatgttgctgaatgggcttctattatgccagataactctgttgatagattcgtcaatccatctaccggtaaattgccagatgatcaaaacatcatttcctccgccattatctctgttaccaacccatattacttgttgcaaaacaccgttggtaagaaggctattagagaagcttcagatgctccattgaacgtcgattctttgtctgttggtggtttggtttctgattcttacgctggtttagttttctgggatgctgatgtttggatgcaacctggtttggtagcttctcatccagaagctgctcaaagagttactaattacagaaccgaaaagtacgctcaagctaaggctaatgctaagactacttttgctggttctaagaacaagacctacgttgaaccatctgctgctgtttatccatggacatctggtagagttggtaactgtactggtactggtccatgttgggattaccaatatcatttgaatggtgacatcggtttgtccttgatctatcaatgggttacttctggtgataccgataccttcagagaaaaacatttcccaatctacgattccgttgctaccttgtactctaatttggttgaacctaatggtacttcttggaccttgactaatatgaccgatccagatgaatacgccaatcatgttgatgctggtggttttactatgccaatgatttctgaaaccttggaatacgctaacgccttcagacaacaatttggtttcgaaatgaacgaaacctggtccgaaattgctgataacgttttgatcttgagagaaaacggtgtcactttggaatacactactatgaatggtactgccgttgttaagcaagcagatgttgttttggctacttacccattggtttacgacaactatacctcccaatcttctttgaccgatttggattactacgccaacaaacaatctgctgatggtccagctatgacttgggctattttttctatagttgctggtgctgtttcaccatctggttgttctgcttatacttaccaacaatactctttcgctccatatgctagagccccatttttccaattgtccgaacaaatgattgatgacgcctctattaacggtggtactcatccagcttatccatttttgacaggtcacggtggtgctaatcaagttgttttgtttggttatttgggtttgagattattgccagacgaagccattcatatcgaacctaatttgccaccacaaatcccacatatcacttacagaattttctattggagaggttggccaatttccgctagatctaattacactcataccgttattcaaagagctgctcacgctccaccattggatactgctgatcatagatttgctaacgcttccattccagtttatgttggtccagaatctaacgctactgtttacactttgccaatcagaagaccattgaccgttcaaaacagacaaatcggtactatcaactccatcccaggtaacttagttcaatgtactccagttttctcccctgatgattttgaaccaggtcaatttccattgtccattgtcgatggtgcaacttctactagatggcaacctaaatctgctaacccatcttctgttaccgttcaattgtctgctgcttcaagacacttgcaaactatggcttcaggtttccattttgaatgggcacaagctccaccagttaatgctactgttatttttcacgaccaaccattgcaaaatccagctttggctttaactgctactccaccaggtgctagaatagttgcttctttaactaacatcaagcaatccttgccatactctgaacaaaccgttgactcaaatcaagtttctttgccagttggtaataccactaccattcaattggatgttccagttcctgtttctagatacgccactttgttgatctctggtaatcaagctttatccggtgctcatgatgatactggtgctacagtagcagaatgggcaattttgggtccaggttctcaagttgatcaaactaagtctactagaaccatgtcctctagagatactgctactttgaagagattgaatagaggtggtggtgccatgattaactac

SEQ ID NO: 32 - protein, 1066 aa, Aspergillus ochraceoroseus
MRVTQLLVKGSGLQSKNLRLSGCMRRHGGGHGIEKTLVGSNNTYQTVFPGVSWDDDHWLLTTTTPDPGHYQSRGSVANGYIGISVSSIGPFFELDMPVDGDVISGWPLFSRRQSFATIAGFYDYQPTTNGSNFPWINQYGGESVISGVPHWSGLIIDLGDETYLDSTVDNQTITGFSSTYDFKAGMLSWSYTWTPAAGDKGSYKITYRIFANKLNVNQAVVDMEIIPSIDSEAIIVNVLDGYAAVRTDFVSSGQDDGAIYSAVRPWGIENVTAYIYANITGSDAVHLSSRRIVTGKAYVNINESSIAQAVDVKFSASEKVRITKFVGAASTDAFSDPQQTAKQAVSQALTAGYLRCLQSHVAEWASIMPDNSVDRFVNPSTGKLPDDQNIISSAIISVTNPYYLLQNTVGKKAIREASDAPLNVDSLSVGGLVSDSYAGLVFWDADVWMQPGLVASHPEAAQRVTNYRTEKYAQAKANAKTTFAGSKNKTYVEPSAAVYPWTSGRVGNCTGTGPCWDYQYHLNGDIGLSLIYQWVTSGDTDTFREKHFPIYDSVATLYSNLVEPNGTSWTLTNMTDPDEYANHVDAGGFTMPMISETLEYANAFRQQFGFEMNETWSEIADNVLILRENGVTLEYTTMNGTAVVKQADVVLATYPLVYDNYTSQSSLTDLDYYANKQSADGPAMTWAIFSIVAGAVSPSGCSAYTYQQYSFAPYARAPFFQLSEQMIDDASINGGTHPAYPFLTGHGGANQVVLFGYLGLRLLPDEAIHIEPNLPPQIPHITYRIFYWRGWPISARSNYTHTVIQRAAHAPPLDTADHRFANASIPVYVGPESNATVYTLPIRRPLTVQNRQIGTINSIPGNLVQCTPVFSPDDFEPGQFPLSIVDGATSTRWQPKSANPSSVTVQLSAASRHLQTMASGFHFEWAQAPPVNATVIFHDQPLQNPALALTATPPGARIVASLTNIKQSLPYSEQTVDSNQVSLPVGNTTTIQLDVPVPVSRYATLLISGNQALSGAHDDTGATVAEWAILGPGSQVDQTKSTRTMSSRDTATLKRLNRGGGAMINY

SEQ ID NO: 33 - DNA, 2115 bp, Rhizoctonia solani
atgagattgaagtttgctttggctgctgcttgtgttttgtctgctagagctttgccacaagctgaaacttctacttctacatctggtacttctactgttactggttctactactgcttccgaacctatttctactgctccagctactgaaactactgctgtttctacagctgttccatctccaactgctccattgggttctccattgccaagacaagctgctttaccaccaaaacaagcttggtgtccatctgaaattttctgcgctggtcaattattgcaatccgttaatttggccaagttgtacgttgattctaagacctttgttgataagccaactgctttcgatgctcaaagagtcttgtctgattttaatgctttgggtccacaagataacgttaccgttggtgctattgctaacttcgtttctaatgactttagaggtgaaggtttggaattggaagctttgactttgtctaacttcccagaaaacccaaccttcttgtccaaaatcaaagatccattggttaaggcctggtccaaaattgtccatacttattggtccgatttgatcagaggtactaatccagaaaccttgtgctctgatagaaatggtactactggttgcgaatcctctttgattccattgaaccatactttcgttgttccaggtggtagattcagagaacaatattactgggattcctactggatcgttagaggtttgttggaatcccaattatacgacatcgttaactccaccttgcaaaacttcatggatgaattggataccatcggtttcattccaaatggtggtagaatctactacttgaacagatctcaaccacctttgttcattcatatgttggctgcttacgtcaacagaactaaggataccgatattttggatagagcattgccattggccgaaaaagaattggcttggtgggctaacaatagaacctttaaagttgaatccccaacctccaaaaagacctacactgtttacagatacgctgttaacaatactgctcctagaccagaatcttacttgccagattacattactgctaacggtgaagatattgaaaccccattgactgatgaacaaaaggctgacttgtatgctgaattggctactggtgctgaatctggttgggattatactgctagatggtctagacaacaattctccggtaacttgtctaacactgaaccacaattgagatccttgaacttgagagctttggttccagttgatttgaacgctatcttgtacggtgctcatattcaattggcatccttgtttgatagacacaccaagtctaagagagatttgagagcaagagcttctgcttcttcatacagaaagaaagctgataccttgaagaaggccattttggatttgtgttggaacgaacaaaagttggccttctacgattttaacactacagctggtggtcaatcttctactttcactgctgctgcattttatccattgtggatgggtatttggcctgaatctttgttgaagtctgaaacaaagactttcggtgccttctcttctgttaactacgttttgaatatgtacaacggtacttacccagctactttcttggaaactggtttacaatgggatttcccaaattcttggccaccacatgtttacattattttggaagccttgaacaacatcccaaagaagttgaacaagcaaaagttgccacaaatcaattctaccgtcacctcttttgatttggttcctgaaggtcaattgggtttgtcagaagatcaattgccaaaacaaaccttggatttgggtggttatgctgccactgatattaacgctggtaacaacacagttatcaacggtggtactccagctaaaaacgaaaagtggagagattctatgaccagacaattggctaacagatatgtttctgctgctttctgttcttggtattctacaggtggttctattcctggtttgttacaacaattgtccccagaagaattgaacgccactaattctgatccatctagtgaaggtcatatgttcgaaaaggtttacttgatccacttcatgttcccaccattggatccacataagtgtcat

SEQ ID NO: 34 - protein, 705 aa, Rhizoctonia solani
MRLKFALAAACVLSARALPQAETSTSTSGTSTVTGSTTASEPISTAPATETTAVSTAVPSPTAPLGSPLPRQAALPPKQAWCPSEIFCAGQLLQSVNLAKLYVDSKTFVDKPTAFDAQRVLSDFNALGPQDNVTVGAIANFVSNDFRGEGLELEALTLSNFPENPTFLSKIKDPLVKAWSKIVHTYWSDLIRGTNPETLCSDRNGTTGCESSLIPLNHTFVVPGGRFREQYYWDSYWIVRGLLESQLYDIVNSTLQNFMDELDTIGFIPNGGRIYYLNRSQPPLFIHMLAAYVNRTKDTDILDRALPLAEKELAWWANNRTFKVESPTSKKTYTVYRYAVNNTAPRPESYLPDYITANGEDIETPLTDEQKADLYAELATGAESGWDYTARWSRQQFSGNLSNTEPQLRSLNLRALVPVDLNAILYGAHIQLASLFDRHTKSKRDLRARASASSYRKKADTLKKAILDLCWNEQKLAFYDFNTTAGGQSSTFTAAAFYPLWMGIWPESLLKSETKTFGAFSSVNYVLNMYNGTYPATFLETGLQWDFPNSWPPHVYIILEALNNIPKKLNKQKLPQINSTVTSFDLVPEGQLGLSEDQLPKQTLDLGGYAATDINAGNNTVINGGTPAKNEKWRDSMTRQLANRYVSAAFCSWYSTGGSIPGLLQQLSPEELNATNSDPSSEGHMFEKVYLIHFMFPPLDPHKCH

SEQ ID NO: 35 - DNA, 1647 bp, Achlya hypogyna
atgttgtccttcagatctttggttactgcttctgctgttttggcttcagctgttgctgctgatattgaacctaaagatatctactgttccggtccagttttggaaactattcaagaagccagattattcaacgactccaagcactttgttgacatggttatgaaggctgctccacaaactgttttgtctgcttttgaagctttgccagatcattctaacactaccttgaaagcctttttggacaagtacttcgatgaaccatctaccgatttggtcgaaattgaattgccagacttcaaagaatctccagctccattgcaagctattaaggatgctgatttgaaagcttgggcattgcaaatcaacaagttgtggaaattattgggtagaaaaagagtcttgccagacggtcattatggttctcatttgccaacaaagcacaacttggttgttccaggtggtagattcagagaatcttattactgggattcctactggatcgttttgggtttgttgaaatctgatatggctgaaactgctaagggtgtcgttcaaaatttgttggatttcgttgatgcctacggttttgttccaaatggtggtagaatctactacttgaacagatctcaaccacctttgttgtccgatatggttagagctattttcgaagctaccaaggatgaagcttatttggctcaagctttaccattattggacaaagaatacgctttctggatgactcaaggtgctgctactcatagagttgaagttcaagctaaagatggtaagacctactccttgaatagatacttttctgctggtacttctccaagaccagaatctttcagagaagatattgaaaccgcttctttggttccagatgcttctagaccaacattataccaaaacattattgctgctgctgaatccggttgggatttttcatcaagatggtttcaagacggtaagactatgaagtccttgtacactactgatgttatcccagttgatttgaacgccatcatgtacagattcgaaagaaacttggcttccttccacaaacacgttggtaatgctcaaaaagcagttgctatggattcagctgctgatgctagaagagctgctattgatgctgttttatggaatgatgctgctggtgcttggaaggattacattacttctgctaaagctcactccaccatcgtttctatttctgattatactccattgtgggctcaagcctttgatgctactgatgcagctagaaatgctagaattttggcctccttgaaatcctctggtttggttttggttgctggtattcaaactactactgctcatactggtcaacaatgggatgcttataatgcttgggctccagaaattgatttcaccgttgaaggtttgttaagattgaacgcttctgaagctacagcttacgctggtaaaatcgtttctgattgggttgctactggtcattctgcttacaaacaaactggttacatgttggaaaagtacaacgcttcagttgttggtggtttgggttctggtggtgaatatgacttgcaattcggtttcggttggactaacggtgttattttgaagttcttgaccgaataccaagacttgttacaacatgatcactgt

SEQ ID NO: 36 - protein, 549 aa, Achlya hypogyna
MLSFRSLVTASAVLASAVAADIEPKDIYCSGPVLETIQEARLFNDSKHFVDMVMKAAPQTVLSAFEALPDHSNTTLKAFLDKYFDEPSTDLVEIELPDFKESPAPLQAIKDADLKAWALQINKLWKLLGRKRVLPDGHYGSHLPTKHNLVVPGGRFRESYYWDSYWIVLGLLKSDMAETAKGVVQNLLDFVDAYGFVPNGGRIYYLNRSQPPLLSDMVRAIFEATKDEAYLAQALPLLDKEYAFWMTQGAATHRVEVQAKDGKTYSLNRYFSAGTSPRPESFREDIETASLVPDASRPTLYQNIIAAAESGWDFSSRWFQDGKTMKSLYTTDVIPVDLNAIMYRFERNLASFHKHVGNAQKAVAMDSAADARRAAIDAVLWNDAAGAWKDYITSAKAHSTIVSISDYTPLWAQAFDATDAARNARILASLKSSGLVLVAGIQTTTAHTGQQWDAYNAWAPEIDFTVEGLLRLNASEATAYAGKIVSDWVATGHSAYKQTGYMLEKYNASVVGGLGSGGEYDLQFGFGWTNGVILKFLTEYQDLLQHDHC

SEQ ID NO: 37 - DNA, 2304 bp, Schizopora paradoxa
atgaagtggttggttttgttgcaagttgctggtggtgttttgtctatgccacaagctggttcttctactgctacttctccaggtatttctactggtgttccatctattactttgtccacttctgttccagctccaactattccattgactaatgatgttccatcacaagctccattgccaccagttcaagcttggtgtccatcaaagattttttgtgcaggttccttgttgcaaaccgttaacgttgcttcattatacgctgatccaaagacctttgttgacaagccaactaatgcttcttctcaaactgttttggctaacttcaatgctttggttgcttctgctggtaactctacttctaacattactgaacaaaccatggtcaacttcgtcgactctaattttagaggtgaaggtttggaattggaagctttggctttgccaaattttactccaaatccaccattcttgcaaaacatcactgatccattgtctaaggcttttgctcaaaccgttcatggtttttggacccaattgattagaggtacaaactcttctaccttgtgtggtgaaggtactaattctggttcttgtgaatctaccttgatcccattgaaccatacttttgttgttccaggtggtagattcagagaacaatattactgggattcctactggatcatccaaggtttggttcaatcccaattattggatattgctaacgccaccttgcaaaatttcatggacgaattggaacaattcggtttcattccaaatggtggtagattgtactacttgaacagatctcaaccacctttgttcatccacatgttgttcgattatgttcaagcctccaacgactcttctattttgactagagctttgccattggccgaaagagaatttgatttttgggctaccaacagaaccttgaacgttacttctcctttcactaacaaaacttaccaagtctctagatacgccgttaacaatactgctccaagaccagaatcttacttgactgattactctacagctaacggtccagatatctctttgaacgaaactcaaaaagaagccttgtacgctgaattggcttctggtgctgaaactggttgggattatactgttagatttgcctctcaaccattcgctggtggtacaaacaacactaatccaattttgagaaccttggccatcagagaaaccattccaatttgtttgaactccatcttgtacaaagcccacgttttgttggcatccttgtattctgaaccattctcctcttctactaataccaccgctaaagaaagagctgcttttcatactggtgctgctgatcaattgaagtccgctattttggatttgttctgggactctaacaagttggctttctacgatttcaacactacctctatgaccagaaactctattttcactaccgctcatttttacccaatgtggaatggtattttcccagatgaattattgtccaacgaaactgctgcttttggtgctttttcctccattaacatggtcatgaacaagttcaacggtactttcccaactaccttcatcgaatctggtttacaatgggatgctccaaatgcttggccaccacatcaattcattgcattgcaagctttacaaaacgtcccaatgaacatttctactaagccagttccagctactccatctggtcaaactgctttttctttgattccttccggtcaattaggtttgtccgaaatgcaattgccaggtcaacctattaagggtggttctaatgcatctgctactgctgatacaaatgctttgaatggtactgttgtcaacggtggtaatgctactggtaatgaaccttggtctgttacattgcaaagagaaatggctaacagatacttcacttctgctttgtgttcttggcatgctacaggtggttcaattcctaatgttttggccagattgtctgatgccgaattggctattacaaactcccaaaacaataccggtaacatgttcgaaaagttctcttactccgatgttgattcttcaggcggtggtggtgaatatactgttcaagcaggttttggttggactaacggtgttgttttgtgggttgcttcaacttatggtaacgttttgaactctccacaatgcccaccattattggtttctacaggttcttcttcttcatctggtggtggtggttcttcaggtggtaattctgcaggtcatagaactgctccagcaccatttgctttagctttagctgctgctatgttggttgcctttattggtatg

SEQ ID NO: 38 - protein, 768 aa, Schizopora paradoxa
MKWLVLLQVAGGVLSMPQAGSSTATSPGISTGVPSITLSTSVPAPTIPLTNDVPSQAPLPPVQAWCPSKIFCAGSLLQTVNVASLYADPKTFVDKPTNASSQTVLANFNALVASAGNSTSNITEQTMVNFVDSNFRGEGLELEALALPNFTPNPPFLQNITDPLSKAFAQTVHGFWTQLIRGTNSSTLCGEGTNSGSCESTLIPLNHTFVVPGGRFREQYYWDSYWIIQGLVQSQLLDIANATLQNFMDELEQFGFIPNGGRLYYLNRSQPPLFIHMLFDYVQASNDSSILTRALPLAEREFDFWATNRTLNVTSPFTNKTYQVSRYAVNNTAPRPESYLTDYSTANGPDISLNETQKEALYAELASGAETGWDYTVRFASQPFAGGTNNTNPILRTLAIRETIPICLNSILYKAHVLLASLYSEPFSSSTNTTAKERAAFHTGAADQLKSAILDLFWDSNKLAFYDFNTTSMTRNSIFTTAHFYPMWNGIFPDELLSNETAAFGAFSSINMVMNKFNGTFPTTFIESGLQWDAPNAWPPHQFIALQALQNVPMNISTKPVPATPSGQTAFSLIPSGQLGLSEMQLPGQPIKGGSNASATADTNALNGTVVNGGNATGNEPWSVTLQREMANRYFTSALCSWHATGGSIPNVLARLSDAELAITNSQNNTGNMFEKFSYSDVDSSGGGGEYTVQAGFGWTNGVVLWVASTYGNVLNSPQCPPLLVSTGSSSSSGGGGSSGGNSAGHRTAPAPFALALAAAMLVAFIGM

SEQ ID NO: 39 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 40 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 41 - protein, 375 aa, Saccharomyces cerevisiae
SKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYDYQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 42 - protein, 292 aa, Bifidobacterium adolescentis
MSEHIFRSTTRHMLRDSKDYVNQTLMGGLSGFESPIGLDRLDRIKALKSGDIGFVHSWDINTSVDGPGTRMTVFMSGCPLRCQYCQNPDTWKMRDGKPVYYEAMVKKIERYADLFKATGGGITFSGGESMMQPAFVSRVFHAAKQMGVHTCLDTSGFLGASYTDDMVDDIDLCLLDVKSGDEETYHKVTGGILQPTIDFGQRLAKAGKKIWVRFVLVPGLTSSEENVENVAKICETFGDALEHIDVLPFHQLGRPKWHMLNIPYPLEDQKGPSAAMKQRVVEQFQSHGFTVY

SEQ ID NO: 43 - protein, 791 aa, Bifidobacterium adolescentis
MAAVDATAVSQEELEAKAWEGFTEGNWQKDIDVRDFIQKNYTPYEGDESFLADATDKTKHLWKYLDDNYLSVERKQRVYDVDTHTPAGIDAFPAGYIDSPEVDNVIVGLQTDVPCKRAMMPNGGWRMVEQAIKEAGKEPDPEIKKIFTKYRKTHNDGVFGVYTKQIKVARHNKILTGLPDAYGRGRIIGDYRRVALYGVNALIKFKQRDKDSIPYRNDFTEPEIEHWIRFREEHDEQIKALKQLINLGNEYGLDLSRPAQTAQEAVQWTYMGYLASVKSQDGAAMSFGRVSTFFDVYFERDLKAGKITETDAQEIIDNLVMKLRIVRFLRTKDYDAIFSGDPYWATWSDAGFGDDGRTMVTKTSFRLLNTLTLEHLGPGPEPNITIFWDPKLPEAYKRFCARISIDTSAIQYESDKEIRSHWGDDAAIACCVSPMRVGKQMQFFAARVNSAKALLYAINGGRDEMTGMQVIDKGVIDPIKPEADGTLDYEKVKANYEKALEWLSETYVMALNIIHYMHDKYAYESIEMALHDKEVYRTLGCGMSGLSIAADSLSACKYAKVYPIYNKDAKTTPGHENEYVEGADDDLIVGYRTEGDFPLYGNDDDRADDIAKWVVSTVMGQVKRLPVYRDAVPTQSILTITSNVEYGKATGAFPSGHKKGTPYAPGANPENGMDSHGMLPSMFSVGKIDYNDALDGISLTNTITPDGLGRDEEERIGNLVGILDAGNGHGLYHANINVLRKEQLEDAVEHPEKYPHLTVRVSGYAVNFVKLTKEQQLDVISRTFHQGAVVD

SEQ ID NO: 44 - protein, 910 aa, Bifidobacterium adolescentis
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYVDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEIPEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 45 - protein, 1098 aa, Saccharomyces cerevisiae
MALIVASLFLPYQPQFELDTSLPENSQVDSSLVNIQAMANDQQQQRALSNNISQESLVAPAPEQGVPPAISRSATRSPSAFNRASSTTNTATLDDLVSSDIFMENLTANATTSHTPTSKTILKPRKNGSVERFFSPSSNIPTDRIASPIQHEHDSGSRIASPIQQQQQDPTANLLKNVNKSLLVHSLLNNTSQTSLEGPNNHIVTPKSRAGNRPTSAATSLVNRTKQGSASSGSSGSSAPPSIKRITPHLTASAAKQRPLLAKQPSNLKYSELADISSSETSSQHNESDPDDLTTAPDEEYVSDLEMDDAKQDYKVPKFGGYSNKSKLKKYALLRSSQELFSRLPWSIVPSIKGNGAMKNAINTAVLENIIPHRHVKWVGTVGIPTDEIPENILANISDSLKDKYDSYPVLTDDVTFKAAYKNYCKQILWPTLHYQIPDNPNSKAFEDHSWKFYRNLNQRFADAIVKIHKKGDTIWIHDYHLMLVPQMVRDVLPFAKIGFTLHVSFPSSEVFRCLAQREKILEGLTGADFVGFQTREYARHFLQTSNRLLMADVVHDEELKYNGRVVSVRFTPVGIDAFDLQSQLKDGSVMQWRQLIRERWQGKKLIVCRDQFDRIRGIHKKLLAYEKFLVENPEYVEKSTLIQICIGSSKDVELERQIMIVVDRINSLSTNISISQPVVFLHQDLDFSQYLALSSEADLFVVSSLREGMNLTCHEFIVCSEDKNAPLLLSEFTGSASLLNDGAIIINPWDTKNFSQAILKGLEMPFDKRRPQWKKLMKDIINNDSTNWIKTSLQDIHISWQFNQEGSKIFKLNTKTLMEDYQSSKKRMFVFNIAEPPSSRMISILNDMTSKGNIVYIMNSFPKPILENLYSRVQNIGLIAENGAYVSLNGVWYNIVDQVDWRNDVAKILEDKVERLPGSYYKINESMIKFHTENAEDQDRVASVIGDAITHINTVFDHRGIHAYVYKNVVSVQQVGLSLSAAQFLFRFYNSASDPLDTSSGQITNIQTPSQQNPSDQEQQPPASPTVSMNHIDFACVSGSSSPVLEPLFKLVNDEASEGQVKAGHAIVYGDATSTYAKEHVNGLNELFTIISRIIED

SEQ ID NO: 46 - protein, 896 aa, Saccharomyces cerevisiae
MTTTAQDNSPKKRQRIINCVTQLPYKIQLGESNDDWKISATTGNSALYSSLEYLQFDSTEYEQHVVGWTGEITRTERNLFTREAKEKPQDLDDDPLYLTKEQINGLTTTLQDHMKSDKEAKTDTTQTAPVTNNVHPVWLLRKNQSRWRNYAEKVIWPTFHYILNPSNEGEQEKNWWYDYVKFNEAYAQKIGEVYRKGDIIWIHDYYLLLLPQLLRMKFNDESIIIGYFHHAPWPSNEYFRCLPRRKQILDGLVGANRICFQNESFSRHFVSSCKRLLDATAKKSKNSSDSDQYQVSVYGGDVLVDSLPIGVNTTQILKDAFTKDIDSKVLSIKQAYQNKKIIIGRDRLDSVRGVVQKLRAFETFLAMYPEWRDQVVLIQVSSPTANRNSPQTIRLEQQVNELVNSINSEYGNLNFSPVQHYYMRIPKDVYLSLLRVADLCLITSVRDGMNTTALEYVTVKSHMSNFLCYGNPLILSEFSGSSNVLKDAIVVNPWDSVAVAKSINMALKLDKEEKSNLESKLWKEVPTIQDWTNKFLSSLKEQASSDDDVERKMTPALNRPVLLENYKQAKRRLFLFDYDGTLTPIVKDPAAAIPSARLYTILQKLCADPHNQIWIISGRDQKFLNKWLGGKLPQLGLSAEHGCFMKDVSCQDWVNLTEKVDMSWQVRVNEVMEEFTTRTPGSFIERKKVALTWHYRRTVPELGEFHAKELKEKLLSFTDDFDLEVMDGKANIEVRPRFVNKGEIVKRLVWHQHGKPQDMLKGISEKLPKDEMPDFVLCLGDDFTDEDMFRQLNTIETCWKEKYPDQKNQWGNYGFYPVTVGSASKKTVAKAHLTDPQQVLETLGLLVGDVSLFQSAGTVDLDSRGHVKNSESSLKSKLASKAYVMKRSASYTGAKV